SEQUENCE LISTING (53 sequences)

SEQ ID NO: 1 - protein, 117 aa, synthetic construct
ESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 2 - protein, 118 aa, synthetic construct
KSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQLMN

SEQ ID NO: 3 - protein, 117 aa, synthetic construct
ESLFKGPRDYNPISSTICHLTNESDGHTTSLYGIGFGPFIITNKHLFRRNNGTLLVQSLHGVFKVKNTTTLQQSLIDGRDMIIIRMPKDFPPFPQKLKFREPQREERICLVTTNFQT

SEQ ID NO: 4 - protein, 118 aa, Synthetic construct
KSMSSMVSDTSCTFPSSDGIFWKHWIQTKDGQCGSPLVSTRDGFIVGIHSASNFTNTNNYFTSVPKNFMELKTNQEAQQWVSGWRLNADSVLWGGHKVFMVKPEEPFQPVKEATQLMN

SEQ ID NO: 5 - protein, 117 aa, Synthetic construct
ESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREHQREERICLVTTNFQT

SEQ ID NO: 6 - protein, 117 aa, Synthetic construct
ESLFKGPRDYNPISSTICHLTNESDGHTTSLYGIGFGPFIITNKHLFRRNNGTLLVQSLHGVFKVKNTTTLQQELIDGRDMIIIRMPKDFPPFPQKLKFREPQREERICLVTTNFQT

SEQ ID NO: 7 - protein, 118 aa, Synthetic construct
KSMSSMVSDTSCTFPSSDGIFWKHWIQTKDGQCGSPLVSPRDGFIVGIHSASNFTNTNNYFTSVPKNFMELLTNQEAQQWVSGWRLNADSVLWGGHKVFMVKPEEPFQPVKEATQLMN

SEQ ID NO: 8 - protein, 7 aa, synthetic construct
ENLYFQM

SEQ ID NO: 9 - protein, 168 aa, Synthetic construct
PKKKRKVSGDALDDFDLDMLGSDALDDFDLDMLGSDALDDFDLDMLGSDALDDFDLDMLGSGGGGSGGGGSGGGGSGTARPGERPFQCRICMRNFSKGERLVRHTRTHTGEKPFQCRICMRNFSRMDNLSTHLRTHTGEKPFQCRICMRNFSRKDALNRHLKTHLRGS

SEQ ID NO: 10-11 - skipped (2 entries)

SEQ ID NO: 12 - DNA, 11 bp, synthetic construct
  source 1..11: mol_type = other DNA
gaaatgagtca

SEQ ID NO: 13 - DNA, 24 bp, synthetic construct
  source 1..24: mol_type = other DNA
aatttcacacctaggtgtgaaatt

SEQ ID NO: 14 - DNA, 30 bp, synthetic construct
  source 1..30: mol_type = other DNA
acgccttctgtatgaaacagtttttcctcc

SEQ ID NO: 15 - protein, 26 aa, synthetic construct
MVSYWDTGVLLCALLSCLLLTGSSSG

SEQ ID NO: 16 - protein, 732 aa, synthetic construct
SKLKDPELSLKGTQHIMQAGQTLHLQCRGEAAHKWSLPEMVSKESERLSITKSACGRNGKQFCSTLTLNTAQANHTGFYSCKYLAVPTSKKKETESAIYIFISDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVQISTPRPVKLLRGHTLVLNCTATTPLNTRVQMTWSYPDEKNKRASVRRRIDQSNSHANIFYSVLTIDKMQNKDKGLYTCRVRSGPSFKSVNTSVHIYDKAFITVKHRKQQVLETVAGKRSYRLSMKVKAFPSPEVVWLKDGLPATEKSARYLTRGYSLIIKDVTEEDAGNYTILLSIKQSNVFKNLTATLIVNVKPQIYEKAVSSFPDPALYPLGSRQILTCTAYGIPQPTIKWFWHPCNHNHSEARCDFCSNNEESFILDADSNMGNRIESITQRMAIIEGKNKMASTLVVADSRISGIYICIASNKVGTVGRNISFYITDVPNGFHVNLEKMPTEGEDLKLSCTVNKFLYRDVTWILLRTVNNRTMHYSISKQKMAITKEHSITLNLTIMNVSLQDSGTYACRARNVYTGEEILQKKEITIRDQEAPYLLRNLSDHTVAISSSTTLDCHANGVPEPQITWFKNNHKIQQEPGIILGPGSSTLFIERVTEEDEGVYHCKATNQKGSVESSAYLTVQGTSDKSNLE

SEQ ID NO: 17 - protein, 22 aa, synthetic construct
LITLTCTCVAATLFWLLLTLFI

SEQ ID NO: 18 - protein, 5 aa, synthetic construct
RKMKR

SEQ ID NO: 19 - protein, 19 aa, synthetic construct
MQSKVLLAVALWLCVETRA

SEQ ID NO: 20 - protein, 745 aa, synthetic construct
ASVGLPSVSLDLPRLSIQKDILTIKANTTLQITCRGQRDLDWLWPNNQSGSEQRVEVTECSDGLFCKTLTIPKVIGNDTGAYKCFYRETDLASVIYVYVQDYRSPFIASVSDQHGVVYITENKNKTVVIPCLGSISNLNVSLCARYPEKRFVPDGNRISWDSKKGFTIPSYMISYAGMVFCEAKINDESYQSIMYIVVVVGYRIYDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRIPAKYLGYPPPEIKWYKNGIPLESNHTIKAGHVLTIMEVSERDTGNYTVILTNPISKEKQSHVVSLVVYVPPQIGEKSLISPVDSYQYGTTQTLTCTVYAIPPPHHIHWYWQLEEECANEPSQAVSVTNPYPCEEWRSVEDFQGGNKIEVNKNQFALIEGKNKTVSTLVIQAANVSALYKCEAVNKVGRGERVISFHVTRGPEITLQPDMQPTEQESVSLWCTADRSTFENLTWYKLGPQPLPIHVGELPTPVCKNLDTLWKLNATMFSNSTNDILIMELKNASLQDQGDYVCLAQDRKTKKRHCVVRQLTVLERVAPTITGNLENQTTSIGESIEVSCTASGNPPPQIMWFKDNETLVEDSGIVLKDGNRNLTIRRVRKEDEGLYTCQACSVLGCAKVEAFFIIEGAQEKTNLE

SEQ ID NO: 21 - protein, 21 aa, synthetic construct
IIILVGTAVIAMFFWLLLVII

SEQ ID NO: 22 - protein, 5 aa, synthetic construct
LRTVK

SEQ ID NO: 23 - protein, 21 aa, synthetic construct
MALPVTALLLPLALLLHAARP

SEQ ID NO: 24 - protein, 19 aa, synthetic construct
MEFGLSWLFLVAILKGVQC

SEQ ID NO: 25 - protein, 21 aa, synthetic construct
LVVVGGVLACYSLLVTVAFII

SEQ ID NO: 26 - protein, 27 aa, synthetic construct
FWVLVVVGGVLACYSLLVTVAFIIFWV

SEQ ID NO: 27 - protein, 9 aa, synthetic construct
RSKRSRLLH

SEQ ID NO: 28 - protein, 21 aa, synthetic construct
LVVVAGVLFCYGLLVTVALCV

SEQ ID NO: 29 - protein, 6 aa, synthetic construct
GGGSGG

SEQ ID NO: 30 - protein, 21 aa, synthetic construct
MLPCLVVLLAALLSLRLGSDA

SEQ ID NO: 31 - protein, 214 aa, synthetic construct
HGTELPSPPSVWFEAEFFHHILHWTPIPNQSESTCYEVALLRYGIESWNSISNCSQTLSYDLTAVTLDLYHSNGYRARVRAVDGSRHSNWTVTNTRFSVDEVTLTVGSVNLEIHNGFILGKIQLPRPKMAPANDTYESIFSHFREYEIAIRKVPGNFTFTHKKVKHENFSLLTSGEVGEFCVQVKPSVASRSNKGMWSKEECISLTRQYFTVTN

SEQ ID NO: 32 - protein, 21 aa, synthetic construct
VIIFFAFVLLLSGALAYCLAL

SEQ ID NO: 33 - protein, 9 aa, synthetic construct
QLYVRRRKK

SEQ ID NO: 34 - protein, 19 aa, synthetic construct
MAWSLGSWLGGCLLVSALG

SEQ ID NO: 35 - protein, 201 aa, synthetic construct
MVPPPENVRMNSVNFKNILQWESPAFAKGNLTFTAQYLSYRIFQDKCMNTTLTECDFSSLSKYGDHTLRVRAEFADEHSDWVNITFCPVDDTIIGPPGMQVEVLADSLHMRFLAPKIENEYETWTMKNVYNSWTYNVQYWKNGTDEKFQITPQYDFEVLRNLEPWTTYCVQVRGFLPDRNKAGEWSEPVCEQTTHDETVPS

SEQ ID NO: 36 - protein, 22 aa, synthetic construct
WMVAVILMASVFMVCLALLGCF

SEQ ID NO: 37 - protein, 9 aa, synthetic construct
ALLWCVYKK

SEQ ID NO: 38 - protein, 33 aa, synthetic construct
MEAAVAAPRPRLLLLVLAAAAAAAAALLPGATA

SEQ ID NO: 39 - protein, 93 aa, synthetic construct
LQCFCHLCTKDNFTCVTDGLCFVSVTETTDKVIHNSMCIAEIDLIPRDRPFVCAPSSKTGSVTTTYCCNQDHCNKIELPTTVKSSPGLGPVEL

SEQ ID NO: 40 - protein, 21 aa, synthetic construct
AAVIAGPVCFVCISLMLMVYI

SEQ ID NO: 41 - protein, 5 aa, synthetic construct
CHNRT

SEQ ID NO: 42 - protein, 22 aa, Synthetic construct
MGRGLLRGLWPLHIVLWTRIAS

SEQ ID NO: 43 - protein, 144 aa, synthetic construct
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQ

SEQ ID NO: 44 - protein, 21 aa, synthetic construct
VTGISLLPPLGVAISVIIIFY

SEQ ID NO: 45 - protein, 5 aa, synthetic construct
CYRVN

SEQ ID NO: 46 - protein, 29 aa, synthetic construct
MGLSTVPDLLLPLVLLELLVGIYPSGVIG

SEQ ID NO: 47 - protein, 182 aa, synthetic construct
LVPHLGDREKRDSVCPQGKYIHPQNNSICCTKCHKGTYLYNDCPGPGQDTDCRECESGSFTASENHLRHCLSCSKCRKEMGQVEISSCTVDRDTVCGCRKNQYRHYWSENLFQCFNCSLCLNGTVHLSCQEKQNTVCTCHAGFFLRENECVSCSNCKKSLECTKLCLPQIENVKGTEDSGTT

SEQ ID NO: 48 - protein, 21 aa, synthetic construct
VLLPLVIFFGLCLLSLLFIGL

SEQ ID NO: 49 - protein, 5 aa, synthetic construct
MYRYQ

SEQ ID NO: 50 - protein, 22 aa, synthetic construct
MAPVAVWAALAVGLELWAAAHA

SEQ ID NO: 51 - protein, 235 aa, synthetic construct
LPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQHTQPTPEPSTAPSTSFLLPMGPSPPAEGSTGD

SEQ ID NO: 52 - protein, 30 aa, synthetic construct
FALPVGLIVGVTALGLLIIGVVNCVIMTQV

SEQ ID NO: 53 - protein, 5 aa, synthetic construct
KKKPL

(19) United States
(12) Patent Application Publication
(10) Pub. No.: US 2025/0313607 A1
(43) Pub. Date: Oct. 9, 2025

(54) SYNTHETIC MODULAR EXTRACELLULAR SENSORS THAT EMPLOY NATURAL RECEPTOR LIGAND-BINDING DOMAINS

(71) Applicant: Northwestern University, Evanston, IL (US)

(72) Inventors: Joshua N. Leonard, Evanston, IL (US); Hailey Ilyse Edelstein, Evanston, IL (US)

(73) Assignee: Northwestern University, Evanston, IL (US)

(21) Appl. No.: 18/865,145

(22) PCT Filed: May 12, 2023
(86) PCT No.: PCT/US2023/022079

§ 371 Date: Nov. 12, 2024

Related U.S. Application Data
(60) Provisional application No. 63/341,916, filed on May 13, 2022.

(51) Int. Cl. C07K 14/705; C07K 14/71; C07K 14/715; G01N 33/542

(52) U.S. Cl. CPC C07K 14/70578; C07K 14/71; C07K 14/7155; G01N 33/542; C07K 2319/02; C07K 2319/03; C07K 2319/50

(57) ABSTRACT

The present disclosure provides synthetic receptor systems for engineering mammalian cell-based devices to sense soluble, physiological cues.

### CROSS-REFERENCE TO RELATED APPLICATION

This application is the U.S. National Stage of PCT/US2023/022079, filed May 12, 2023, which claims priority to U.S. Provisional Application No. 63/341,916, filed May 13, 2022, the contents of which are incorporated herein by reference in their entireties.

The instant application contains a Sequence Listing which has been submitted in XML format and is hereby incorporated by reference in its entirety. Said XML copy, created on Jul. 13, 2023, is named 121384-0201.xml and is 49,400 bytes.

### STATEMENT OF GOVERNMENT SUPPORT

This invention was made with government support under Grant No. EB026510 awarded by the National Institutes of Health (NIH), National Institute of Biomedical Imaging and Engineering. The United States government has certain rights in the invention.

### TECHNICAL FIELD

The present disclosure relates generally to the field of synthetic biosensors. More specifically, the present disclosure relates synthetic receptor systems for engineering mammalian cell-based devices to sense soluble, physiological cues.

### BACKGROUND

The following description of the background of the present technology is provided simply as an aid in understanding the present technology and is not admitted to describe or constitute prior art to the present technology.

Early demonstrations of genetically engineering customized functions in mammalian cells indicate a vast potential to benefit applications including directed stem cell differentiation and cancer immunotherapy. In general, most applications require precise control of gene expression and the capability to sense and respond to external cues. Despite the growing availability of biological parts (such as libraries of promoters and regulatory proteins) that could be used to control cell states, assembling parts to compose customized receptors that function as intended remains a challenge.

The present disclosure provides novel synthetic biosensors that can be used in a variety of cell engineering platforms and other applications.

### SUMMARY

Described herein are synthetic receptor systems for engineering mammalian cell-based devices to sense soluble, physiological cues. More specifically, the present disclosure provides new receptor systems that combine a natural ectodomain with existing Modular Extracellular Sensor Architecture (MESA) to produce highly selective and useful cell biosensors and systems.

In one aspect, the present disclosure provides biosensors comprising a protein dimer comprising a first protein and a second protein that each comprises:
(a) an extracellular ligand-binding domain of a human receptor protein,
(b) a transmembrane domain,
(c) a juxtamembrane domain comprising 5-12 amino acids connected to a cytoplasmic end of the transmembrane domain, and
(d) an intracellular dimerizing domain;
wherein the intracellular dimerizing domain of the first protein comprises a half of a split protease; and
wherein the intracellular dimerizing domain of the second protein comprises (i) the complementary half of the split protease, (ii) a protease cleavage site (PCS), and (iii) a transcription factor linked thereto, such that the split protease components reconstitute upon dimerization of the first protein and the second protein, cleaving the PCS and releasing the transcription factor.

In some embodiments, the first protein, the second protein, or both further comprise a signal peptide of the human receptor protein, which is, optionally, derived from a human CD8a receptor or a human IgG variable heavy chain.

In some embodiments, the extracellular domain of the human receptor protein, the transmembrane domain, and the juxtamembrane domain are all derived from the same human protein. In some embodiments, the extracellular domain of the human receptor protein, the transmembrane domain, and the juxtamembrane domain are derived from at least two different human proteins.

In some embodiments, the transmembrane domain is derived from a murine or human CD28 receptor. In some embodiments, the juxtamembrane domain comprises a flexible repeated sequence of glycine and serine amino acids.

In some embodiments, the extracellular domain of the human receptor protein binds to transforming growth factor beta (TGF-β), a tumor necrosis factor (TNF), an interleukin, or vascular endothelial growth factor (VEGF).

In some embodiments, the extracellular domain of the human receptor protein is an extracellular domain of TGF-β receptor 1 (TGF-βR1) or TGF-β receptor 2 (TGF-βR2). In some embodiments, the extracellular domain of the human receptor protein is an extracellular domain of TNF receptor 1 (TNFR1) or TNF receptor 2 (TNFR2). In some embodiments, the extracellular domain of the human receptor protein is an extracellular domain of interleukin-10 receptor b (IL-10Rb) or interleukin-10 receptor a (IL-10Ra). In some embodiments, the extracellular domain of the human receptor protein is an extracellular domain of VEGF receptor 1 (VEGFR1) or VEGF receptor 2 (VEGFR2).

In some embodiments, the first protein comprises the N-terminal half of split tobacco etch virus protease and the second protein comprises the complementary C-terminal half of split tobacco etch virus protease, a protease cleavage site (PCS), and a transcription factor. In some embodiments, the first protein comprises the C-terminal half of split tobacco etch virus protease and the second protein comprises the complementary N-terminal half of split tobacco etch virus protease, a protease cleavage site (PCS), and a transcription factor. In some embodiments, the N-terminal half of split tobacco etch virus protease comprises SEQ ID NO: 1, 3, 5, or 6. In some embodiments, the C-terminal half of split tobacco etch virus protease comprises SEQ ID NO: 2, 4, or 7.

In some embodiments, the transcription factor is a synthetic transcription (synTF) factor or a naturally occurring transcription factor.

The foregoing general description and following detailed description are exemplary and explanatory and are intended to provide further explanation of the disclosure as claimed. Other objects, advantages, and novel features will be readily apparent to those skilled in the art from the following brief description of the drawings and detailed description of the disclosure.

### DESCRIPTION OF THE FIGURES

FIGS. 1A-1C show how natural ectodomain (NatE) MESA receptors are designed to rewire natural receptor ectodomain binding of soluble ligands through custom transcriptional output. (FIG. 1A) Schematic of the Modular Extracellular Sensor Architecture (MESA). (FIG. 1B) Schematic illustrating the conversion of a natural receptor into a NatE MESA receptor, highlighting how natural receptor ectodomains, transmembrane domains, and juxtamembrane domains are incorporated. (FIG. 1C) Shows a schematic of the panel of ligands targeted by tested NatE MESA systems described in this disclosure and the natural human receptors that were incorporated. From left to right, classification of each system and its receptor and ligand multimeric states, overview of what is currently known about the natural receptor mechanisms for each system, and proposed NatE MESA mechanism for each system. Abbreviations: ECDs, ectodomains; ICDs, intracellular domains; NTEVp, N-terminal component of split tobacco etch virus protease; CTEVp, C-terminal component of split tobacco etch virus protease; TF, transcription factor.

FIGS. 2A-2C show the general flow cytometry gating strategies used. (FIG. 2A) Illustrates the flow cytometry gating strategy used to identify single HEK293FT cells for a representative sample of cells. (FIG. 2B) Illustrates the flow cytometry gating strategy used to identify cells that are expressing miRFP720 from the landing pad locus (for identifying reporter cells with an accessible landing pad locus cells). A gate is drawn on unmodified HEK293FT cells (left) to include <0.1% of cells in the Alexa750 fluorescence channel. This gate encompasses most landing pad-modified cells (right). A similar approach is used with the FITC channel to identify transposon modified HEK293FT cells that are constitutively expressing mNeonGreen (not shown). (FIG. 2C) Calibration of fluorescence intensities to absolute units requires inclusion of a sample of Spherotech UltraRainbow Calibration Particles (URCP) in each experiment. These beads have nine fluorescent bead populations. Beads are identified based on the FSC-A vs. SSC-A profile (left). For each experiment, two fluorescent channels were used to identify all nine bead populations (right). The mean fluorescence intensities (MFIs) of each population in the relevant channel(s) are exported and plotted against manufacturer-provided absolute values of fluorophores per bead for each population (for example, Molecules of Equivalent Fluorescein MEFLs for mNeonGreen, Molecules of Equivalent PE-TexasRed MEPTRs for DsRedExpress2). To generate the calibration curve, a linear regression was performed with the constraint that the y-intercept equals zero. This calibration is done for each experiment, and then exported MFI values (which have arbitrary fluorescence units) are converted to absolute units using the multiplier obtained from the regression. Error from the linear regression is also appropriate propagated.

FIGS. 3A-3D show the schematic of the workflow for converting natural receptors into synthetic NatE MESA biosensors. (FIG. 3A) Workflow with black outlined boxes and black numbers depicts process as it was implemented in this study. This workflow employs a strategy to down select receptor variants based on surface and whole cell expression before testing function. (FIGS. 3B-D) Colored arrows and corresponding numbers depict alternative workflows depending on desired properties and throughput. (FIG. 3B) The blue workflow prioritizes testing receptor function (in co-expressed ligand setup with most optimal receptor-ligand interactions for signaling) before characterizing expression properties and doing so only for select variants or to explain functional differences. (FIG. 3C) The orange workflow prioritizes testing receptor function first in the translational context (with genomically integrated receptors and external, recombinant ligand) and only evaluating expression properties for functional hits, while the green workflow (FIG. 3D) only focuses on biosensor function in the translational context (with genomically integrated receptors and external, recombinant ligand). The alternative workflows might facilitate higher throughput explorations because surface expression and western blotting can limit throughput, though they provide useful information about which receptor variants have desired properties of surface expression and correct protein size. All workflows start with considerations for defining the receptor design space and end with options for tuning performance.

FIG. 4 shows an overview of the consistent design choices explored across all four receptor systems included in this study. Some systems employed additional design choices as needed (including split TEVp mutants and human CD28 TMD variations), which are not covered here.

FIGS. 5A-5C show conversions of VEGFR into a VEGF-sensing NatE MESA receptor system and analysis. (FIG. 5A) Schematic of generalized VEGFR signaling mechanism highlighting receptor interactions (top). Schematic of the proposed converted VEGFR-based NatE MESA signaling mechanism (bottom). (FIG. 5B) Surface expression of each single chain transfected in HEK293FT cells alone via staining for 3×FLAG epitope. Histograms show data for transfected (fluorescent) cells and the gray histograms in each column are transfection controls (no receptor). Mean APC fluorescence intensities are listed. (FIG. 5C) Whole-cell expression of each VEGFR NatE MESA receptor was measured via western blotting of a 3×FLAG epitope tag fused to the N-terminus of receptors. Soluble mNeonGreen (mNG) or mTagBFP2 (mTB2) were included as negative controls because each receptor-expressing plasmid is a poly-transfection vector that also contains either a constitutively expressed mNG or mTB2 in the backbone. A rapamycin-sensing MESA receptor, pPD810 was included as an internal control for 3×FLAG-tagged protein expression.

FIGS. 6A-6C show conversions of VEGFR into a VEGF-sensing NatE MESA receptor system and analysis continued. (FIG. 6A) Functional assay of transfected VEGFR-based NatE MESA receptors across all mixed and matched ECDs, TMDs, and signaling domain pairings with and without two isoforms of co-expressed human VEGF. Many pairs demonstrate ligand-inducible signaling, with fold induction (induced signal/background signal) shown below the plot. Some receptor pairs are more sensitive to one VEGF isoform over another. All receptors include the native signal sequence that matches the ECD. (FIG. 6B) The reporter cell line was transfected with each receptor chain individually to assess single receptor induced reporter expression in the absence of ligand. No reporter expression is observed with NTEVp receptors, which do not contain a synTF. Minimal reporter expression is observed with CTEVp receptors from spontaneous release of the synTF from the membrane. In addition, the reporter cell line was transfected with VEGF only to show that no reporter expression is induced only in the presence of VEGF. (FIG. 6C) Evaluation of receptor signal sequence effect on reporter expression with transfected receptors and co-expressed VEGF. Two inducible pairs from the initial panel were selected and pairwise combinations of receptors with varying signal sequences were evaluated in the presence and absence of VEGF. Fold induction values are indicated below each pair of bars for each receptor combination, indicating the ratio between reporter expression in the presence of and absence of ligand. Both receptor pairs were inducible across all signal sequence pairings and signal sequence choice impacted both background and induced expression (two-way ANOVA, p<0.05). Each bar represents the mean of the mean of transfected cells across three biologic replicates and error bars indicate standard error of the mean (S.E.M).

FIGS. 7A-7E show conversions of VEGFR into a VEGF-sensing NatE MESA receptor system and analysis continued. (FIG. 7A) Functional assay with transfected VEGFR-based NatE MESA receptors treated with and without recombinant, external ligand. (FIG. 7B) Reporter expression of transfected receptors when transfected cells were treated with external, recombinant VEGF. The eight inducible pairs from the functional assay involving co-expressed, internal VEGF were selected. All receptors contain their natural signal sequence. Fold inductions are shown below each pair of bars. Each bar represents the mean of transfected cells across three biologic replicates and error bars indicate standard error of the mean (S.E.M). (FIG. 7C) Surface expression of receptors with different NTEVp and CTEVp mutant domains was measured via immunohistochemistry. Receptors with the trans-cleavage MESA mechanism (full TEVp on one chain, protease recognition sequence and synTF on the other chain) were included as a reference to show that splitting the TEVp does not ablate surface expression. Histograms included all transfected cells based on expression of a constitutive fluorescent protein in the receptor-encoding plasmid backbone. (FIG. 7D) Transfected receptor combinations with varying split TEVp mutants yielding a range of interfacial energies were tested for functionality with co-expressed and external VEGF. Darker bars indicate higher interfacial energy, resulting in lower propensity for TEVp reconstitution. High performing receptors with co-expressed ligand (left) also detected external ligand (right). Fold inductions are shown below each pair of bars. Each bar represents the mean of transfected cells across three biologic replicates and error bars indicate standard error of the mean (S.E.M). (FIG. 7E) Post-hoc analysis of cells transfected with VEGFR NatE MESA receptors and treated with external VEGF (100 ng/ml). Expression of each single receptor chain was quantified via a proxy fluorescent protein included in the receptor-encoding plasmid under a separate constitutive promoter. Cells were binned based on the expression level of each fluorescent proxy and fold induction was calculated for each bin (ratio of reporter expression in the presence and absence of VEGF). Darker bins represent areas of expression with higher fold inductions. Bins where the cell count was below 100 were grayed out and fold induction was not calculated.

FIGS. 8A-8F show conversions of VEGFR into a VEGF-sensing NatE MESA receptor system and analysis continued. (FIG. 8A) Schematics detailing the composition of the PiggyBac transposon vectors used to generate stable cell lines expressing VEGFR NatE MESA receptors. For the “all-in-one” vector, the reporter, two receptor chains, and selection marker were all included in the same transposon vector and used to transduce HEK293FT cells. For the “reporter-less” vector, the reporter was excluded, and the vector was used to transduce the previously engineered HEK293FT landing pad reporter cells. In both vector designs, transcriptional units (containing the promoter, the gene, and the terminator) are flanked by a pair of chicken hypersensitive site 4 (cHS4) insulators. (FIG. 8B) Functional assay with HEK293FT cells that contain genomically-integrated VEGFR-based NatE MESA receptors and recombinant, external ligand. HEK293FTs were modified with PiggyBac transposons with receptors and a reporter in the transposon (top) or with just receptors into a cell line that contains a genomic reporter in the AAVS1 locus (bottom). Two receptor pairings were investigated: A CTEVp (190K) receptor with a VEGFR1 signal sequence, ECD, and TMD paired with a NTEVp (75S or WT) receptor with a VEGFR2 signal sequence, ECD, and TMD. The wild type NTEVp pairing shows significant inducibility in the all-in-one transposon design (two-tailed Welch's t test, p<0.05). Both transposon designs also contain a constitutively expressed mNeonGreen and puromycin resistance marker to facilitate antibiotic selection and fluorescence-based identification of engineered cells. Data shown includes all mNeonGreen+ cells. (FIG. 8C) Post-hoc analysis of silencing effects on all-in-one transposon cell lines with the CTEVp 190K, NTEVp wt receptor pair. Reporter expression as well as percent of cells with an active reporter (defined by non-engineered HEK293FTs) were compared across three different experiments (with 1 being the earliest and 3 being the latest), with and without exogenous VEGF. Silencing of the reporter increases and responsiveness to ligand decrease over time. Each bar represents the mean across three biologic replicates and error bars indicate standard error of the mean (S.E.M). (FIG. 8D) Sodium butyrate (NaB) was employed to reverse silencing. Data show the effect of NaB concentration and incubation time on VEGF-independent reporter expression in the “all-in-one” VEGFR NatE MESA cell line. (FIG. 8E) Effect of NaB concentration and incubation time on receptor surface expression as measured via 3×FLAG epitope tag. Note that this is total receptor expression-NTEVp and CTEVp receptor expression cannot be differentiated because they contain the same 3×FLAG epitope tag. (FIG. 8F) Effect of NaB concentration and incubation time on reporter expression in the presence and absence of transfected VEGF (left). Fold inductions (ratio of reporter expression in the presence and absence of VEGF) were plotted as a function of NaB concentration and incubation time (right). Each bar or data point represents the mean across three biologic replicates and error bars indicate standard error of the mean (S.E.M).

FIGS. 9A-9B show conversions of VEGFR into a VEGF-sensing NatE MESA receptor system and analysis continued. (FIG. 9A) Recombinant VEGF dose response with HEK293FT cells that contain a genomically integrated receptor pair with the wild type NTEVp tested in FIG. 8B. A dose-dependent increase in reporter expression is observed until 100 ng/mL, after which reporter expression decreases, likely due to ligand toxicity. (FIG. 9B) Functional assay with HEK293FT cells that contain a genomically integrated receptor pair with the wild type NTEVp senses surface-bound VEGF. For bar graphs, bars represent the mean of three biological replicates of either transfected cells or transposon modified (mNeonGreen+) cells and error bars depict standard error of the mean (S.E.M.).

FIGS. 10A-10C show conversions of IL-10R into an IL-10-sensing NatE MESA receptor system and analysis. (FIG. 10A) Schematic of generalized IL-10R signaling mechanism highlighting receptor interactions (top). Schematic of the proposed converted IL-10R-based NatE MESA signaling mechanism (bottom). (FIG. 10B) Surface expression of each single chain transfected in HEK293FT cells alone via staining for 3×FLAG epitope. Histograms show data for transfected (fluorescent) cells and the gray histograms in each column are transfection controls (no receptor). Mean APC fluorescence intensities are listed. (FIG. 10C) Whole-cell expression of each IL-10R NatE MESA receptor was measured via western blotting of a 3×FLAG epitope tag fused to the N-terminus of receptors. Soluble mNeonGreen (mNG) or mTagBFP2 (mTB2) were included as negative controls (−) because each receptor-expressing plasmid is a poly-transfection vector that also contains either a constitutively expressed mNG or mTB2 in the backbone. A rapamycin-sensing MESA receptor, pPD810 was included as an internal control for 3×FLAG-tagged protein expression (+).

FIGS. 11A-11D show conversions of IL-10R into an IL-10-sensing NatE MESA receptor system and analysis continued. (FIG. 11A) Functional assay of transfected IL-10R-based NatE MESA receptors across all mixed and matched ECDs, TMDs, and signaling domain pairings with and without co-expressed human IL-10. Mainly heteroassociative pairs (mismatched ECDs) demonstrate ligand-inducible signaling, with fold inductions (FI, induced signal/background signal) shown below the plot. CD28 mediates increased background signal. All receptors include the native signal sequence that matches the ECD. (FIG. 11B) The reporter cell line was transfected with each receptor individually to assess single receptor-induced reporter expression in the absence of ligand. No reporter expression is observed with NTEVp receptors, which don't contain a synTF. Minimal reporter expression is observed with CTEVp receptors from spontaneous release of the synTF from the membrane. The y-axis is scaled to match that of (FIG. 11A). (FIG. 11C) The reporter cell line was transfected with IL-10 only to show that no reporter expression is induced only in the presence of IL-10. Each bar represents the mean of transfected cells across three biologic replicates and error bars indicate standard error of the mean (S.E.M). (FIG. 11D) Evaluation of the effect of NatE MESA receptor signal sequence on reporter expression with transfected receptors and co-expressed IL-10. One inducible ECD configuration from the initial panel was selected (NTEVp-IL-10Rb and CTEVp-IL-10Ra) and pairwise combinations of receptors with varying signal sequences and TMDs were evaluated in the presence and absence of co-expressed IL-10. Fold induction (FI) values are indicated below each pair of bars for each receptor combination, indicating the ratio between reporter expression in the presence of and absence of ligand. All receptor pairs were inducible across all signal sequence and transmembrane domain pairings and signal sequence choice impacted both background and induced expression (two-way ANOVA, p<0.05). The use of a CD28-based transmembrane domain on both receptors in a pair leads to substantially higher background signal. The bottom plot shows that expression of each single CTEVp chain alone drives minimal reporter expression. Each bar represents the mean of transfected cells across three biologic replicates and error bars indicate standard error of the mean (S.E.M).

FIGS. 12A-12D show conversions of IL-10R into an IL-10-sensing NatE MESA receptor system and analysis continued. (FIG. 12A) Functional assay with transfected IL-10R-based NatE MESA receptors treated with and without recombinant, external ligand at early and late time points when assay timing was extended to enable more receptor expression and reporter expression to build up. Ligand induced a significant increase in reporter expression over the non-treated condition for all treatment timings (two-tailed Welch's t test, p<0.05). (FIG. 12B) Post-hoc transfection analysis of cells transfected with IL-10R NatE MESA receptors and treated with external IL-10 (250 ng/ml). Expression of each single receptor chain was quantified via a proxy fluorescent protein included in the receptor-encoding plasmid under a separate constitutive promoter. Cells were binned based on the expression level of each fluorescent proxy and both reporter output (the geometric mean of fluorescence intensity, GMFI) and fold induction (ratio of geometric mean of reporter expression in the presence and absence of IL-10) were calculated for each bin. Bins where the cell count was below 100 are blacked out and were not analyzed. Highest inducibility is observed with bins with cells that received the most of both receptors (upper right-hand corner). (FIG. 12C) Functional assay with HEK293FT cells that contain genomically-integrated IL-10R-based NatE MESA receptors and recombinant, external ligand. HEK293FTs were modified with PiggyBac transposons with receptors and a reporter in the transposon (top) or with just receptors into a cell line that contains a genomic reporter in the AAVS1 locus (bottom). Two receptor pairings were investigated: A CTEVp receptor with an IL-10Ra signal sequence, ECD, and TMD paired with a NTEVp receptor with an IL-10Rb ECD, and TMD and either a hIgG VH or hCD8a signal sequence. The CD8a signal sequence NTEVp pairing shows more inducibility across both transposon designs. Both transposon designs also contain a constitutively expressed mNeonGreen and puromycin resistance marker to facilitate antibiotic selection and fluorescence-based identification of engineered cells (not shown in diagrams here but shown in detailed schematics in (FIG. 12D). Data shown includes all mNeonGreen+ cells. Ligand induced a significant increase in reporter expression over the non-treated condition for all receptor-expressing cell lines except for the cell line containing the inert receptors (two-tailed Welch's t test, p<0.05). (FIG. 12C) Schematics detailing the composition of the transposon vectors used to generate stable cell lines expressing IL-10R NatE MESA receptors. For the “all-in-one” vector, the reporter, both receptors, and a selection marker were all included in the same transposon vector and used to transduce HEK293FT cells. For the “reporter-less” vector, the reporter was excluded, and the vector was used to transduce the previously engineered HEK293FT landing pad reporter cells. In both vector designs, transcriptional units (containing the promoter, the gene, and the terminator) are flanked by a pair of chicken hypersensitive site 4 (cHS4) insulators.

FIGS. 13A-13D shows conversion of IL-10R into an IL-10-sensing NatE MESA receptor system and analysis continued. (FIG. 13A) Stable cell lines engineered with two IL-1OR NatE MESA receptor pairs (which differ in the signal sequence on the NTEVp receptor) and with two different transposon types were cultured with and without exogenous human IL-10 to evaluate ability to sense external ligand. Two cell lines that contain receptors that do not sense IL-10 (inert receptors in this assay) were also included as negative controls to show that external IL-10 ligand itself does not activate the reporter. Each bar represents the mean of mNeonGreen+ cells (constitutive transposon marker) across three biologic replicates and error bars indicate standard error of the mean (S.E.M). (FIG. 13B) Recombinant IL-10 dose response with HEK293FT cells that contain a genomically integrated receptor pair with the CD8a signal sequence NTEVp tested. A dose-dependent increase in reporter expression is observed until 250 ng/mL, after which reporter expression plateaus, likely due to ligand toxicity. Ligand-induced reporter expression is significantly different from the untreated condition down to 16 ng/mL (one-way ANOVA, p<0.05). Analogous dose responses for the same receptor design when implemented via Sleeping Beauty transposon and when the cell line is sorted for top mNeonGreen expressers is shown in FIG. 16B. For all bar graphs and scatter plots, bars and points represent the mean across transfected cells or transposon modified (mNeonGreen+) cells and error bars depict standard error of the mean (S.E.M.). (FIG. 13C) Microscopy images after 48 h of culture of the cell line used in panel (FIG. 13B) with 250 ng/mL IL-10. mNeonGreen fluorescence and brightfield indicate where cells and transposon-engineered cells are. DsRedExpress2 fluorescence corresponds to reporter expression. Plot shows mean reporter expression normalized to mean mNeonGreen fluorescence, averaged across three fields of view (three different wells) quantified for images collected every two hours. After 14 hours, the ligand-treated conditions are significantly different than the non-ligand treated conditions (multi factor ANOVA, p<0.05). Error bars represent the standard error across three fields of view for each time point. (FIG. 13D) Stable cell lines engineered with two IL-10R NatE MESA receptor pairs (which differ in the signal sequence on the NTEVp receptor) via a Piggy Bac transposon can detect IL-10 in conditioned media from HEK293FT cells engineered to secrete human IL-10. Each bar represents the mean of mNeonGreen+ cells (constitutive transposon marker) across three biologic replicates and error bars indicate standard error of the mean (S.E.M).

FIGS. 14A-14D show conversions of IL-10R into an IL-10-sensing NatE MESA receptor system and analysis continued. (FIG. 14A) A sort strategy was devised to evaluate how different transposon copy numbers contribute to bulk population signaling with IL-10R NatE MESA receptors stably integrated in HEK293FTs. The distribution of mNeonGreen expression (constitutive transposon marker) was broken into eight octiles that each contained 12.5% of the total mNeonGreen+ cells, and the top 4 octiles were isolated. One week after sorting, the expanded octile populations retain differences in mNeonGreen expression. The four octiles were cultured with and without external IL-10 to evaluate (FIG. 14B) inducibility and (FIG. 14C) percent of cells expressing any reporter (defined by non-engineered HEK293FTs). Both reporter expression and percent of cells signaling decreases as octile number increases (mNeonGreen expression decreases), but fold induction is not substantially changed. Each bar represents the mean of mNeonGreen+ cells (constitutive transposon marker) across three biologic replicates and error bars indicate standard error of the mean (S.E.M). (FIG. 14D) The distribution of reporter expression across each expanded octile population shows that in general, cells that are dimly expressing reporter in the absence of ligand express more reporter when the ligand is present. This is evidenced by a growth in the shoulder of the histograms as opposed to a full population shift.

FIGS. 15A-15D show conversions of IL-10R into an IL-10-sensing NatE MESA receptor system and analysis continued. (FIG. 15A) A sort strategy was devised to evaluate both how different transposon copy numbers contribute to bulk population signaling and if a low-background population could be isolated with IL-10R NatE MESA receptors stably integrated in HEK293FTs. First, a quadrant gate was applied to identify low reporter expressing cells (cells were not treated with ligand before this sort) and the upper half of mNeonGreen expression (constitutive transposon marker). From here, mNeonGreen expression was broken into quartiles that each contained 12.5% of the total mNeonGreen+ cells (25% of the top half of mNeonGreen+ cells) and isolated. One week after sorting, the expanded octile populations retain differences in mNeonGreen expression. The four octiles were cultured with and without external IL-10 to evaluate (FIG. 15B) inducibility and (FIG. 15C) percent of cells expressing any reporter (defined by non-engineered HEK293FTs). Both reporter expression and percent of cells signaling decreases as octile number increases (mNeonGreen expression decreases), but fold induction is not substantially changed. Compared to sorted populations in FIG. 14A-14D, overall signal and percent of signaling cells are much lower. Each bar represents the mean of mNeonGreen+ cells (constitutive transposon marker) across three biologic replicates and error bars indicate standard error of the mean (S.E.M). (FIG. 15D) The distribution of reporter expression across each expanded octile population shows that in general, cells that are dimly expressing reporter in the absence of ligand express more reporter when the ligand is present. This is evidenced by a growth in the shoulder of the histograms as opposed to a full population shift.

FIGS. 16A-16B show conversions of IL-10R into an IL-10-sensing NatE MESA receptor system and analysis continued. (FIG. 16A) To evaluate if reversal of silencing could increase the proportion of cells that can signal, stable cells transposon-engineered cells with reporter only or with reporter and IL-10R NatE MESA receptors were treated with 1 mM NaB for 48 h before being treated with or without external IL-10. Background signal, induced signal, and percent of cells expressing reporter (defined by non-engineered HEK293FTs) increased with NaB treatment (two-tailed Welch's t-test, p<0.05). Each bar represents the mean of mNeonGreen+ cells (constitutive transposon marker) across three biologic replicates and error bars indicate standard error of the mean (S.E.M). (FIG. 16B) Reporter expression dose response data for stable cells engineered with the reporter and the IL-10R NatE MESA receptor pair with CD8a signal sequence NTEVp receptor via a Sleeping Beauty transposon (antibiotic selected cells) and Piggy Bac transposon (antibiotic selected cells and top mNeonGreen octile). Though signal magnitude changes across these cell lines, fold induction (ratio of induced to background signal) is relatively conserved. Each point represents the mean of mNeonGreen+ cells (constitutive transposon marker) across three biologic replicates and error bars indicate standard error of the mean (S.E.M).

FIGS. 17A-17E show conversions of TGF-βR into a TGF-β-sensing NatE MESA receptor system and analysis. (FIG. 17A) Schematic of generalized TGF-βR signaling mechanism highlighting receptor interactions (top). Schematic of the proposed converted TGF-βR-based NatE MESA signaling mechanism (bottom). (FIG. 17B) Surface expression of each single chain transfected in HEK293FT cells alone. Histograms show data for transfected (fluorescent) cells and the gray histograms in each column are transfection controls (no receptor). Mean APC fluorescence intensity for each sample are listed. (FIG. 17C) Whole-cell expression of each TGF-βR NatE MESA receptor was measured via western blotting of a 3×FLAG epitope tag fused to the N-terminus of receptors. Soluble mNeonGreen (mNG) or mTagBFP2 (mTB2) were included as negative controls (−) because each receptor-expressing plasmid is a poly-transfection vector that also contains either a constitutively expressed mNG or mTB2 in the backbone. A rapamycin-sensing MESA receptor, pPD810 was included as an internal control for 3×FLAG-tagged protein expression (+). Letters indicate receptor variants labeled in more detail in FIG. 17B. (FIG. 17D) Functional assay of transfected TGF-βR-based NatE MESA receptors across all mixed and matched ECDs, TMDs, and signaling domain pairings with and without co-expressed human TGF-βR. CD28 mediates increased background signal, and most configurations show low signal, with a moderate increase in signal when two TGF-βRI ECDs are employed. Single CTEVp chains transfected alone were also tested and produced minimal reporter output (right). (FIG. 17E) To validate that CTEVp receptors could be cleaved by a reconstituted TEVp, we co-transfected each chain with or without soluble TEVp and found that all receptors could release synTFs to induce reporter expression in the presence of TEVp.

FIGS. 18A-18B show conversions of TGF-βR into a TGF-β-sensing NatE MESA receptor system and analysis continued. (FIG. 18A) Because TGF-βR-based NatE MESA receptors demonstrated poorer surface expression that other systems explored in this study, to improve surface expression, we evaluated if switching to a human CD28 transmembrane domain with either the same truncated length used in other receptors employing a mouse CD28 transmembrane domain in this study, the full domain, or the full domain plus juxtamembrane domain. In general, changing the CD28 transmembrane domain variant did not substantially change surface expression. ECD and signal sequence have the biggest impact on surface expression. Histograms show data for transfected (fluorescent) cells and the gray histograms in each column are transfection controls (no receptor). Mean APC fluorescence intensity for each sample are listed. (FIG. 18B) Functional assay of transfected TGF-βR-based NatE MESA receptors across different CD28 transmembrane domain variations. All pairs are not inducible with co-expressed TGF-β1. Again, pairings with two TGF-βRI ECDs produced the highest signal. For all bar graphs, bars represent the mean across transfected cells of three biologic replicates and error bars depict standard error of the mean (S.E.M.).

FIGS. 19A-19E show conversions of TNFR into a TNF-sensing NatE MESA receptor system and analysis. (FIG. 19A) Schematic of generalized TNFR signaling mechanism highlighting receptor interactions (top). Schematic of the proposed converted TNFR-based NatE MESA signaling mechanism (bottom). (FIG. 19B) Surface expression of each single chain transfected in HEK293FT cells alone. Histograms show data for transfected (fluorescent) cells and the gray histograms in each column are transfection controls (no receptor). Mean APC fluorescent intensity for each sample is listed. (FIG. 19C) Whole-cell expression of each TNFR NatE MESA receptor was measured via western blotting of a 3×FLAG epitope tag fused to the N-terminus of receptors. Soluble mNeonGreen (mNG) or mTagBFP2 (mTB2) were included as negative controls (−) because each receptor-expressing plasmid is a poly-transfection vector that also contains either a constitutively expressed mNG or mTB2 in the backbone. A rapamycin-sensing MESA receptor, pPD810 was included as an internal control for 3×FLAG-tagged protein expression (+). Letters indicate receptor variants labeled in more detail in FIG. 19B. (FIG. 19D) To validate bioactivity of a TNF co-expression system, we co-transfected TNF-encoding plasmids into a previously validated stable cell line with a genomically integrated fluorescent NF-kB reporter. In HEK293FTs, TNF would be expected to activate a NF-kB reporter through endogenous receptors. The three expression systems included proTNF (membrane bound version that is reflective of how natural TNF is produced before cleavage), and mature TNF that includes only the soluble portion with either a mouse or human Ig κ light chain leader sequence for secretion. We proceeded with mature TNF with the human Ig κ light chain leader sequence. (FIG. 19E) Functional assay of transfected TNFR-based NatE MESA receptors across all mixed and matched ECDs, TMDs, and signaling domain pairings with and without co-expressed human TNF. CD28 mediates increased background signal, and most configurations show low signal, with a moderate increase in signal when two TNFR1 ECDs are employed. Single CTEVp chains transfected alone were also tested and produced minimal reporter output (right).

FIGS. 20A-20G show conversions of TNFR into a TNF-sensing NatE MESA receptor system and analysis continued. (FIG. 20A) To validate that CTEVp receptors could be cleaved by a reconstituted TEVp, we co-transfected each chain with or without soluble TEVp and found that all receptors could release synTFs to induce reporter expression in the presence of TEVp. (FIG. 20B) Removal of a MMP cleavage site was removed from TNFR1-based receptors to evaluate if reporter output could be increased. Surface expression to detect the 3×FLAG epitope tag was unchanged with these mutations. Histograms included all transfected cells based on expression of a constitutive fluorescent protein in the receptor-encoding plasmid backbone. (FIG. 20C) Reporter output was also unchanged with mutations to remove a MMP cleavage site. (FIG. 20D) Surface expression to detect the 3×FLAG epitope tag of receptors with different NTEVp and CTEVp mutant domains was measured. Histograms included all transfected cells based on expression of a constitutive fluorescent protein in the receptor-encoding plasmid backbone. (FIG. 20E) Functional assay of transfected TNFR-based NatE MESA receptors across different NTEVp/CTEVp mutant pairings with and without co-expressed human TNF. Receptor pairs with NTEVp/CTEVp pairings with low interfacial energy conferred higher, moderately inducible signaling. All receptors contain their native signal sequence and transmembrane domains. Interfacial energy is measured in Rosetta energy units (REUs). (FIG. 20F) Receptor pairings that employ a trans-cleavage signaling mechanism with a full TEVp on one chain and a recognition sequence on the other chain, which do not have to reconstitute the TEVp to signal, also displayed higher signaling. These receptors contain their native signal sequence and transmembrane domains. (FIG. 20G) Functional assay with transfected TNFR-based NatE MESA receptors treated with and without recombinant, external ligand and co-transfected with TNF. All receptors contain a CD28 transmembrane domain. A transfection assay with extended timing (late ligand addition after a passage post transfection) shows low inducibility for the TNFR2-TNFR2 pairing (multi factor ANOVA, p<0.05). For all bar graphs, bars represent the mean across transfected cells of three biologic replicates and error bars depict standard error of the mean (S.E.M.).

FIGS. 21A-21E show the use of IL-10R-based NatE MESA receptors to drive translationally relevant outputs. (FIG. 21A) Functional assay with IL-10R-based NatE MESA receptors integrated genomically in Jurkats via PiggyBac transposon. Cells can sense 250 ng/mL external, recombinant ligand and produce synTF-driven reporter output. (FIG. 21B) The same cells used in FIG. 21A can sense IL-10 dose-dependently in the antibiotic-selected cells down to 16 ng/mL and in a population sorted for top mNeonGreen expressers down to 2 ng/ml (one-way ANOVA, p<0.05). mNeonGreen is a constitutive marker in the transposon, along with a puromycin resistance marker to facilitate selection and sorting. Detailed schematics are shown in FIG. 23. (FIG. 21C) Fold induction is unchanged after sorting, showing that these dose responses are characterizing inherent features of the receptors themselves in this experimental setup, independent of context features. (FIG. 21D) HEK293FTs express a HER2-targeting CAR in an IL-10-dependent manner with both transfected, co-expressed IL-10 and recombinant IL-10. (FIG. 21E) Western blot showing expression of membrane-bound IL-15 (mbIL-15) in HEK293FTs. Sample descriptions for each lane are on the right. Arrow markers for relevant protein sizes are also shown. The expected dominant band size for mbIL-15 is 73 kDa including post-translational modifications (PTMs). A C-terminal myc epitope tag was included on the mbIL-15 for detection. A consistent band for endogenous myc protein expression appears in all lanes. Lanes A-D show that co-transfected IL-10 and syn-TF lead to an increase in mbIL-15 expression when the biosensor circuit is transfected on a plasmid. Lanes G-L show that treatment with recombinant IL-10 or co-transfection with IL-10 and syn-TF in cells with stably expressed biosensor circuits yield an increase in mbIL-15 expression. Lanes E, F, M, N are negative controls.

FIGS. 22A-22G show the use of IL-10R-based NatE MESA receptors to drive translationally relevant outputs continued. (FIG. 22A) Surface expression of IL-10R NatE MESA CTEVp receptors with different natural transcription factors (BATF, cJun, and Tbet) was measured via immunohistochemistry to detect a surface expressed 3×FLAG epitope tag on the N-terminus of each receptor. Histograms include all transfected cells based on expression of a constitutive transfection control fluorescent protein on a separate co-transfected plasmid and the negative control is a sample transfected with just the transfection control plasmid. Mean APC fluorescence intensity for each sample is listed. (FIG. 22B) Whole-cell expression of each IL-10R NatE MESA receptor was measured via western blotting of the 3×FLAG epitope tag fused to the N-terminus of receptors. With each receptor, a plasmid encoding mTagBFP2 was included as a transfection control, so soluble mTagBFP2 was included alone as a negative control (−). A rapamycin-sensing MESA receptor, pPD810 was included as an internal control for 3×FLAG-tagged protein expression (+). Natural transcription factor-containing receptors produced a band at the expected size and the BATF-containing receptor produced a prominent, small cleavage product. (FIG. 22C) Synthetic promoters were engineered to detect release of natural transcription factors. Six copies of binding sites for each transcription factor were placed upstream of a YB_TATA minimal promoter. These reporter systems were validated by co-transfecting three doses of transcription factor-encoding plasmid along with each reporter plasmid in HEK293FTs. The level of background reporter output from transfection of the reporter plasmid alone varies across designs because HEK293FTs express varying levels of cJun and BATF but not Tbet endogenously. Each bar represents the mean of transfected cells across three biologic replicates and error bars indicate standard error of the mean (S.E.M). (FIG. 22D) IL-10R-based NatE MESA receptors can release natural TFs from the membrane as measured by reading out signal of engineered natural TF-responsive fluorescent reporters. Co-expression of each natural TF induced a significant change in reporter expression for each reporter in HEK293FTs (two-tailed Welch's t-test, p<0.05). These reporters were validated in FIG. 22C. (FIG. 22E) IL-10R-based NatE MESA receptors can release active cJun from the membrane in response to external ligand when genomically integrated in Jurkats. Ligand induced a significant change in reporter expression after 96 h for receptors that release cJun, but not for the other natural TFs in this context or for unmodified Jurkats (two-tailed Welch's t-test, p<0.05). For all bar graphs, bars represent the mean across transfected or transposon-modified (mNeonGreen+) cells of three biological replicates and error bars depict standard error of the mean (S.E.M.). (FIG. 22F) Jurkats engineered with natural transcription factor containing IL-10R NatE MESA receptors were cultured with or without external IL-10 for 48 h to assay synthetic reporter output from engineered natural transcription factor reporters. This is the earlier timepoint data that corresponds to data shown in (FIG. 22E). Ligand treatments did not produce statistically significant changes in reporter expression in 48 h. (FIG. 22G) Jurkats engineered with just the synthetic reporters for each natural transcription factor were cultured with or without external IL-10 for 48 h to assay how IL-10 alone (no receptors) impacts synthetic reporter output. Ligand treatments did not produce statistically significant changes in reporter expression. Each bar represents the mean of mNeonGreen+ cells (constitutive transposon marker) across three biologic replicates and error bars indicate standard error of the mean (S.E.M).

FIG. 23 shows the use of IL-10R-based NatE MESA receptors to drive translationally relevant outputs continued. Schematics detailing the composition of the Piggy Bac transposon vectors used to generate stable cell lines expressing IL-10R NatE MESA receptors in Jurkats and HEK293FTs. In all vector designs, transcriptional units (containing the promoter, the gene, and the terminator) are flanked by a pair of cHS4 insulators.

FIGS. 24A-24E show multiplexing NatE MESA receptors to sense and respond to multiple inputs. (FIG. 24A) Cross-reactivity assay of VEGF and IL-10 NatE MESA receptors when co-transfected in a HEK293FT stable reporter cell line. A VEGFR NTEVp chain was co-transfected with an IL-10R CTEVp chain, and vice versa. Co-expressed ligands were included as indicated, with empty vector filler DNA transfected for no ligand conditions. The VEGFR NTEVp receptor can have transient interactions with the IL-10R CTEVp receptor, resulting in release of the synTF form the membrane and subsequent reporter expression. See FIG. 24B for a full panel of receptor design choice combinations. (FIG. 24B) Cross-reactivity evaluation between VEGFR and IL-10R NatE MESA receptors when expressed in the same cells. Two VEGFR NTEVp receptors were co-transfected with one IL-10R CTEVp receptor, and two IL-10R NTEVp receptors were co-transfected with one VEGFR CTEVp receptor in HEK293FTs. Reporter expression was evaluated for conditions with no ligand, each ligand individually (co-expressed VEGF or IL-10), and both ligands. All receptors include their native signal sequence as well as their native transmembrane domain. Each bar represents the mean of transfected cells across three biologic replicates and error bars indicate standard error of the mean (S.E.M). (FIG. 24C) OR gate schematic, implementation, and desired behavior. Each receptor system releases the same synTF upon ligand binding, which can then bind to a cognate promoter and induce reporter expression. The system should be active when either of the inputs (IL-10 and/or VEGF) are present. (FIG. 24D) OR gate functional assay. IL-10R and VEGFR NatE MESA receptors were co-transfected into the reporter cell line, along with co-expressed ligands as indicated (no ligand conditions were transfected with empty vector filler DNA). The heatmap and bar graph show the same data. OR gate showed the desired behavior, with IL-10R driving most of the reporter signal. (FIG. 24E) Full panel of OR gate design choices. VEGFR and IL-10R receptor pairs were co-transfected in HEK293FTs along with the different co-expressed ligands (or empty vector filler DNA in the no ligand condition) and reporter expression was measured. The figure shows the combination of the VEGFR pair with NTEVp mutant 75S and the IL-10R pair with NTEVp signal sequence hIgG VH (first combination on this plot). CTEVp chains were not varied between each of the receptor pairs. Each bar represents the mean of transfected cells across three biologic replicates and error bars indicate standard error of the mean (S.E.M).

FIGS. 25A-25E show multiplexing NatE MESA receptors to sense and respond to multiple inputs continued. (FIG. 25A) Hybrid promoter AND gate schematic, implementation, and desired behavior. Each receptor system releases a different synTF upon ligand binding, which can then bind to a cognate hybrid promoter and induce reporter expression. The system should be active when both inputs are present (IL-10 and VEGF). (FIG. 25B) Synthetic hybrid promoter development for AND gate testing. Four promoter architectures (P1-P4) were built and inserted into the AAVS1 safe harbor locus of Landing Pad cells (HEK293FT-LP). The promoters have interspersed binding sites for COMET synTFs ZF1 and ZF6, with a total of either 6 or 12 total binding sites. Soluble transcription factors were transfected into the engineered cell lines and reporter expression was measured to evaluate the synergistic potential of the hybrid promoter architectures. Synergy is defined here as the ratio of (reporter output with both synTFs-reporter output with no synTFs) to (sum of reporter output with each individual synTF). Each bar represents the mean of transfected cells across three biologic replicates and error bars indicate standard error of the mean (S.E.M). (FIG. 25C) Multi-gene expression vector (MGEV) design for the evaluation of AND gate logic. Each receptor system (VEGFR and IL-10R) was encoded on a different MGEV containing both the NTEVp and CTEVp chains, as well as a fluorescent proxy (mNeonGreen for VEGFR, mTagBFP2 for IL-10R). All transcriptional units are controlled by a human EF1α promoter. (FIG. 25D) Hybrid promoter AND gate functional assay. Two promoter architectures were integrated genomically into HEK293FT-LP cells and transfected with multi-gene expression vectors for each receptor system. Co-expressed ligands were included as indicated, with empty vector filler DNA supplemented in no ligand conditions to keep constant total DNA mass. Heatmaps and bar graphs show the same data. Different combinations of which receptor system drives release of each synTF were tested, and some of them showed better synergy than others (though all displayed AND gate behavior). Synergy is defined here as the ratio of (reporter output with both synTFs-reporter output with no synTFs) to (sum of reporter output with each individual synTF). (FIG. 25E) Full panel of hybrid promoter AND gate design choices. VEGFR and IL-10R MGEVs (FIG. 25C) were co-transfected in the engineered P2 and P4 reporter cell lines along with the different co-expressed ligands (or empty vector filler DNA in the no ligand condition). Reporter expression was measured for a panel of design choices. The figure shows the combination of the VEGFR pair with the NTEVp mutant 75S and the IL-10R pair with the NTEVp signal sequence hIgG VH. CTEVp chains were not varied between each of the receptor pairs. Quantification of synergy was included, which was calculated as the background (no ligand condition) subtracted reporter expression when both ligands are present, over the sum of the background subtracted reporter expression when each ligand is present individually. Each bar represents the mean of transfected cells across three biologic replicates and error bars indicate standard error of the mean (S.E.M).

FIGS. 26A-26C show multiplexing NatE MESA receptors to sense and respond to multiple inputs continued. (FIG. 26A) Split-intein synTF AND gate schematic, implementation, and desired behavior. Each receptor system releases a different synTF upon ligand binding, which can then bind to a cognate promoter and induce expression of one half of a split-intein synTF (activation domain fused to intN, DNA-binding domain fused to intC¹⁰⁴), these can then reconstitute and induce reporter expression. The system should be active when both inputs are present (IL-10 and VEGF). (FIG. 26B) Split-intein synTF AND gate functional assay. Two reporter setups were integrated genomically into HEK293FT Landing Pad cells and transfected with multi-gene expression vectors for each receptor system. Co-expressed ligands were included as indicated, with empty vector filler DNA transfected for no ligand conditions. Heatmaps and bar graphs show the same data. Different combinations of what receptor system drives expression of what half of the split-intein synTF were tested, and some of them showed better synergy than others (though most combinations displayed AND gate behavior). For all bar graphs, bars represent the mean of three biological replicates of transfected cells and error bars depict standard error of the mean (S.E.M.). (FIG. 26C) Full panel of split intein synTF AND gate design choices. VEGFR and IL-10R MGEVs were co-transfected in the engineered reporter cell lines along with the different co-expressed ligands (or empty vector filler DNA in the no ligand condition). Reporter expression was measured for a panel of design choices. The figure shows the combination of the VEGFR pair with the NTEVp mutant 75S and the IL-10R pair with the NTEVp signal sequence hIgG VH. CTEVp chains were not varied between each of the receptor pairs. Quantification of synergy was included, which was calculated as the background (no ligand condition) subtracted reporter expression when both ligands are present, over the sum of the background subtracted reporter expression when each ligand is present individually. Each bar represents the mean of transfected cells across three biologic replicates and error bars indicate standard error of the mean (S.E.M).

### DETAILED DESCRIPTION

Cell-based therapies represent an exciting frontier in design-driven medicine, leveraging the natural capabilities of cells to sense, process information, and produce and secrete therapeutic molecules in situ. Synthetic receptor systems enable engineered mammalian cell-based therapies to sense physiological cues and produce therapeutic responses. There now exist examples of synthetic receptor systems that can sense surface-bound and soluble extracellular targets and signal through either natural signaling pathways or synthetic gene circuits.

Synthetic sensors have the advantage of minimally disturbing or being regulated by native cellular processes, yet it remains laborious to generate new synthetic receptors for soluble ligands of interest. Although natural receptors exist for many soluble ligands, no systematic strategy has been developed to convert natural receptors into synthetic receptors that signal orthogonally from native pathways. Towards addressing this goal, the present disclosure shows how natural receptor domains and their corresponding biophysical mechanisms can be leveraged and incorporated into a synthetic receptor architecture, particularly by employing the Modular Extracellular Sensor Architecture (MESA), a synthetic receptor system that signals via proteolytic release of a transcription factor upon receptor dimerization. This signaling mechanism enables customized transcriptional output upon detection of the target ligand. The present inventors systematically characterized surface expression and signaling performance for MESA receptors derived from three different types of human cytokine receptors. This process generated multiple novel, high performing synthetic cytokine receptors. The present inventors also identified mechanisms that render this conversion from natural to synthetic receptors challenging or infeasible, thereby allowing for novel synthetic receptor construction. Thus, the present disclosure provides synthetic sensors for cell-based therapies, diagnostics, and tools for studying disease pathology.

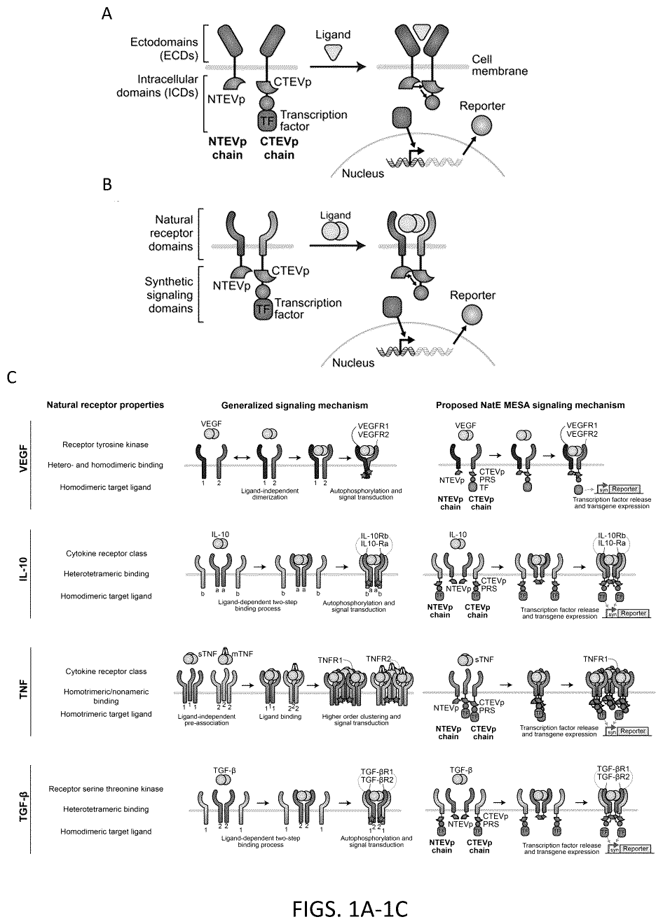

This technology encompasses several synthetic cytokine receptor systems for engineering mammalian cell-based devices to sense soluble, physiological cues. Specifically, this technology employs natural receptor domains within the Modular Extracellular Sensor Architecture (MESA) framework to coopt native receptor-ligand binding mechanisms into user defined transcriptional output. MESA receptors comprise transmembrane proteins that are engineered to release a sequestered transcription factor through proteolytic cleavage upon receptor binding to the target ligand (FIG. 1A) (Daringer, et al. ACS Synthetic Biology, 2014; Dolberg, et al. Nature Chemical Biology, 2021). In some embodiments, termed Natural Ectodomain (NatE) MESA, natural receptor ectodomains mediate ligand binding and signaling output is produced via COMET (Composable Mammalian Elements of Transcription) transcription factors (Donahue, et al. Nature Communications, 2020). Thus, the disclosed receptors rely on conversion of natural receptors, which signal through a variety of biophysical mechanisms and native signaling pathways, into NatE MESA synthetic receptors, which signal through user-defined transcriptional programs. Altogether the disclosed technology enables expedient engineering of high surface-expressing synthetic receptors for sensing extracellular, physiological cues.

### I. DEFINITIONS

It is to be understood that the terminology used herein is for the purpose of describing particular embodiments only, and is not intended to be limiting.

Technical and scientific terms used herein have the meanings commonly understood by one of ordinary skill in the art, unless otherwise defined. Unless otherwise specified, materials and/or methodologies known to those of ordinary skill in the art can be utilized in carrying out the methods described herein, based on the guidance provided herein.

As used herein, the singular terms “a,” “an,” and “the” include plural referents unless the context clearly dictates otherwise. Reference to an object in the singular is not intended to mean “one and only one” unless explicitly so stated, but rather “one or more.”

As used herein, “about” when used with a numerical value means the numerical value stated as well as plus or minus 10% of the numerical value. For example, “about 10” should be understood as both “10” and “9-11.”

As used herein, a phrase in the form “A/B” or in the form “A and/or B” means (A), (B), or (A and B); a phrase in the form “at least one of A, B, and C” means (A), (B), (C), (A and B), (A and C), (B and C), or (A, B, and C).

As used herein, the terms “include” and “including” have the same meaning as the terms “comprise” and “comprising” in that these latter terms are “open” transitional terms that do not limit claims only to the recited elements succeeding these transitional terms. The term “consisting of,” while encompassed by the term “comprising,” should be interpreted as a “closed” transitional term that limits claims only to the recited elements succeeding this transitional term. The term “consisting essentially of,” while encompassed by the term “comprising,” should be interpreted as a “partially closed” transitional term which permits additional elements succeeding this transitional term, but only if those additional elements do not materially affect the basic and novel characteristics of the claim.

As used herein, the terms “protein” or “polypeptide” or “peptide” may be used interchangeable to refer to a polymer of amino acids. Typically, a “polypeptide” or “protein” is defined as a longer polymer of amino acids, of a length typically of greater than 50, 60, 70, 80, 90, or 100 amino acids. A “peptide” is defined as a short polymer of amino acids, of a length typically of 50, 40, 30, 20 or less amino acids.

A “protein” as contemplated herein typically comprises a polymer of naturally or non-naturally occurring amino acids (e.g., alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine). The proteins contemplated herein may be further modified in vitro or in vivo to include non-amino acid moieties. These modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine.

The proteins disclosed herein may include “wild type” proteins and variants, mutants, and derivatives thereof. As used herein the term “wild type” is a term of the art understood by skilled persons and means the typical form of an organism, strain, gene or characteristic as it occurs in nature as distinguished from mutant or variant forms. As used herein, a “variant, “mutant,” or “derivative” refers to a protein molecule having an amino acid sequence that differs from a reference protein or polypeptide molecule. A variant or mutant may have one or more insertions, deletions, or substitutions of an amino acid residue relative to a reference molecule. A variant or mutant may include a fragment of a reference molecule. For example, a mutant or variant molecule may one or more insertions, deletions, or substitution of at least one amino acid residue relative to a reference polypeptide.

Regarding proteins, a “deletion” refers to a change in the amino acid sequence that results in the absence of one or more amino acid residues. A deletion may remove at least 1, 2, 3, 4, 5, 10, 20, 50, 100, 200, or more amino acids residues. A deletion may include an internal deletion and/or a terminal deletion (e.g., an N-terminal truncation, a C-terminal truncation or both of a reference polypeptide). A “variant,” “mutant,” or “derivative” of a reference polypeptide sequence may include a deletion relative to the reference polypeptide sequence.

Regarding proteins, “fragment” is a portion of an amino acid sequence which is identical in sequence to but shorter in length than a reference sequence. A fragment may comprise up to the entire length of the reference sequence, minus at least one amino acid residue. For example, a fragment may comprise from 5 to 1000 contiguous amino acid residues of a reference polypeptide, respectively. In some embodiments, a fragment may comprise at least 5, 10, 15, 20, 25, 30, 40, 50, 60, 70, 80, 90, 100, 150, 250, or 500 contiguous amino acid residues of a reference polypeptide. Fragments may be preferentially selected from certain regions of a molecule. The term “at least a fragment” encompasses the full-length polypeptide. A fragment may include an N-terminal truncation, a C-terminal truncation, or both truncations relative to the full-length protein. A “variant,” “mutant,” or “derivative” of a reference polypeptide sequence may include a fragment of the reference polypeptide sequence.

The term “half” is used herein to define a portion of a protein, “split protein”, or nucleic acid sequence that encodes a protein or split protein, wherein the protein or sequence is divided into two parts. The term “half” is non-limiting, in that it does not necessarily defined as being 50% of the split protein. In some instances, half may be any portion, fragment, or percent of the protein. For example, in some embodiments, half of the split protein may comprise 1%, 5%, 10%, 25%, 50%, 75%, 90%, 95% or 99% of the protein. In some embodiments, the split protein may be divided in two halves, such that the first half comprises 1% and the second half comprises 99% of the protein. In some embodiments, the split protein may be divided in two halves, such that the first half comprises 5% and the second half comprises 95% of the protein. In some embodiments, the split protein may be divided in two halves, such that the first half comprises 10% and the second half comprises 90% of the protein. In some embodiments, the split protein may be divided in two halves, such that the first half comprises 25% and the second half comprises 75% of the protein. In some embodiments, the split protein may be divided in two halves, such that the first half comprises 50% and the second half comprises 50% of the protein.

Regarding proteins, the words “insertion” and “addition” refer to changes in an amino acid sequence resulting in the addition of one or more amino acid residues. An insertion or addition may refer to 1, 2, 3, 4, 5, 10, 20, 30, 40, 50, 60, 70, 80, 90, 100, 150, 200, or more amino acid residues. A “variant,” “mutant,” or “derivative” of a reference polypeptide sequence may include an insertion or addition relative to the reference polypeptide sequence. A variant of a protein may have N-terminal insertions, C-terminal insertions, internal insertions, or any combination of N-terminal insertions, C-terminal insertions, and internal insertions.

Regarding proteins, the phrases “percent identity” and “% identity,” refer to the percentage of residue matches between at least two amino acid sequences aligned sing a standardized algorithm. Methods of amino acid sequence alignment are well-known. Some alignment methods take into account conservative amino acid substitutions. Such conservative substitutions, explained in more detail below, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastp,” that is used to align a known amino acid sequence with other amino acids sequences from a variety of databases.

Regarding proteins, percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

Regarding proteins, the amino acid sequences of variants, mutants, or derivatives as contemplated herein may include conservative amino acid substitutions relative to a reference amino acid sequence. For example, a variant, mutant, or derivative protein may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide.

Conservative amino acid substitutions generally maintain (a) the structure of the polypeptide backbone in the area of the substitution, for example, as a beta sheet or alpha helical conformation, (b) the charge or hydrophobicity of the molecule at the site of the substitution, and/or (c) the bulk of the side chain. Non-conservative amino acids typically disrupt (a) the structure of the polypeptide backbone in the area of the substitution, for example, as a beta sheet or alpha helical conformation, (b) the charge or hydrophobicity of the molecule at the site of the substitution, and/or (c) the bulk of the side chain.

The disclosed proteins, mutants, variants, or described herein may have one or more functional or biological activities exhibited by a reference polypeptide (e.g., one or more functional or biological activities exhibited by wild-type protein).

The disclosed proteins may be substantially isolated or purified. The term “substantially isolated or purified” refers to proteins that are removed from their natural environment, and are at least 60% free, preferably at least 75% free, and more preferably at least 90% free, even more preferably at least 95% free from other components with which they are naturally associated.

Also disclosed herein are polynucleotides, for example polynucleotide sequences that encode proteins or polypeptides as disclosed herein. The terms “polynucleotide,” “polynucleotide sequence,” “nucleic acid” and “nucleic acid sequence” refer to a nucleotide, oligonucleotide, polynucleotide (which terms may be used interchangeably), or any fragment thereof. These phrases also refer to DNA or RNA of genomic, natural, or synthetic origin (which may be single-stranded or double-stranded and may represent the sense or the antisense strand).

Regarding polynucleotide sequences, the terms “percent identity” and “% identity” refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Regarding polynucleotide sequences, percent identity may be measured over the length of an entire defined polynucleotide sequence, for example, as defined by a particular SEQ ID number, or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length supported by the sequences shown herein, in the tables, figures, or Sequence Listing, may be used to describe a length over which percentage identity may be measured.

Regarding polynucleotide sequences, “variant,” “mutant,” or “derivative” may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174:247-250). Such a pair of nucleic acids may show, for example, at least 60%, at least 70%, at least 80%, at least 85%, at least 90%, at least 91%, at least 92%, at least 93%, at least 94%, at least 95%, at least 96%, at least 97%, at least 98%, or at least 99% or greater sequence identity over a certain defined length.

Nucleic acid sequences that do not show a high degree of identity may nevertheless encode similar amino acid sequences due to the degeneracy of the genetic code where multiple codons may encode for a single amino acid. It is understood that changes in a nucleic acid sequence can be made using this degeneracy to produce multiple nucleic acid sequences that all encode substantially the same protein. For example, polynucleotide sequences as contemplated herein may encode a protein and may be codon-optimized for expression in a particular host. In the art, codon usage frequency tables have been prepared for a number of host organisms including humans, mouse, rat, pig, E. coli, plants, and other host cells.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques known in the art. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

The nucleic acids disclosed herein may be “substantially isolated or purified.” The term “substantially isolated or purified” refers to a nucleic acid that is removed from its natural environment, and is at least 60% free, preferably at least 75% free, and more preferably at least 90% free, even more preferably at least 95% free from other components with which it is naturally associated.

“Transformation” or “transfection” describes a process by which exogenous nucleic acid (e.g., DNA or RNA) is introduced into a recipient cell. Transformation or transfection may occur under natural or artificial conditions according to various methods well known in the art, and may rely on any known method for the insertion of foreign nucleic acid sequences into a prokaryotic or eukaryotic host cell. The method for transformation or transfection is selected based on the type of host cell being transformed and may include, but is not limited to, bacteriophage or viral infection or non-viral delivery. Methods of non-viral delivery of nucleic acids include lipofection, nucleofection, microinjection, electroporation, heat shock, particle bombardment, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration). The term “transformed cells” or “transfected cells” includes stably transformed or transfected cells in which the inserted DNA is capable of replication either as an autonomously replicating plasmid or as part of the host chromosome, as well as transiently transformed or transfected cells which express the inserted DNA or RNA for limited periods of time.

The polynucleotide sequences contemplated herein may be present in expression vectors. For example, the vectors may comprise: (a) a polynucleotide encoding an ORF of a protein; (b) a polynucleotide that expresses an RNA that directs RNA-mediated binding, nicking, and/or cleaving of a target DNA sequence; and both (a) and (b). The polynucleotide present in the vector may be operably linked to a prokaryotic or eukaryotic promoter. “Operably linked” refers to the situation in which a first nucleic acid sequence is placed in a functional relationship with a second nucleic acid sequence. For instance, a promoter is operably linked to a coding sequence if the promoter affects the transcription or expression of the coding sequence. Operably linked DNA sequences may be in close proximity or contiguous and, where necessary to join two protein coding regions, in the same reading frame. Vectors contemplated herein may comprise a heterologous promoter (e.g., a eukaryotic or prokaryotic promoter) operably linked to a polynucleotide that encodes a protein. A “heterologous promoter” refers to a promoter that is not the native or endogenous promoter for the protein or RNA that is being expressed.

As used herein, “expression” refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

The term “vector” refers to some means by which nucleic acid (e.g., DNA) can be introduced into a host organism or host tissue. There are various types of vectors including plasmid vector, bacteriophage vectors, cosmid vectors, bacterial vectors, and viral vectors. As used herein, a “vector” may refers to a recombinant nucleic acid that has been engineered to express a heterologous polypeptide (e.g., the fusion proteins disclosed herein). The recombinant nucleic acid typically includes cis-acting elements for expression of the heterologous polypeptide.

Any of the conventional vectors used for expression in eukaryotic cells may be used for directly introducing DNA into a subject. Expression vectors containing regulatory elements from eukaryotic viruses may be used in eukaryotic expression vectors (e.g., vectors containing SV40, CMV, or retroviral promoters or enhancers). Exemplary vectors include those that express proteins under the direction of such promoters as the SV40 early promoter, SV40 later promoter, metallothionein promoter, human cytomegalovirus promoter, murine mammary tumor virus promoter, and Rous sarcoma virus promoter. Expression vectors as contemplated herein may include eukaryotic or prokaryotic control sequences that modulate expression of a heterologous protein (e.g. the fusion protein disclosed herein).

The presently disclosed methods may include delivering one or more polynucleotides, such as or one or more vectors as described herein and/or one or proteins transcribed therefrom, to a host cell. Further contemplated are host cells produced by such methods, and organisms (such as animals, plants, or fungi) comprising or produced from such cells. Conventional viral and non-viral based gene transfer methods can be used to introduce nucleic acids in mammalian cells or target tissues. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell.

In the methods contemplated herein, a host cell may be transiently or non-transiently transfected (i.e., stably transfected) with one or more vectors described herein. In some embodiments, a cell is transfected as it naturally occurs in a subject (i.e., in situ). In some embodiments, a cell that is transfected is taken from a subject (i.e., explanted). In some embodiments, the cell is derived from cells taken from a subject, such as a cell line. Suitable cells may include stem cells (e.g., embryonic stem cells and pluripotent stem cells). A cell transfected with one or more vectors described herein may be used to establish a new cell line comprising one or more vector-derived sequences. In the methods contemplated herein, a cell may be transiently transfected with the components of a system as described herein (such as by transient transfection of one or more vectors, or transfection with RNA), and modified through the activity of a complex, in order to establish a new cell line comprising cells containing the modification but lacking any other exogenous sequence.

As used herein “receptor” refers to naturally occurring or endogenous proteins that are associated with the cell membrane (e.g., membrane bound proteins, integral membrane proteins, transmembrane proteins, glycophosphatidylinosital anchored proteins) and have binding specificity for a cognate ligand, and to proteins having an amino acid sequence which is the same as that of a naturally occurring or endogenous receptor protein (e.g., recombinant proteins, synthetic proteins (i.e., produced using the methods of synthetic organic chemistry)). Accordingly, as defined herein, the term includes mature receptor protein, naturally occurring polymorphic or allelic variants, and other naturally occurring isoforms of a receptor (e.g., produced by alternative splicing or other cellular processes), and modified (e.g. post-translational modifications, lipidated, glycosylated) or unmodified forms of the foregoing. Alternative splicing of RNA encoding a receptor may yield several isoforms of the receptor that differ in the number of amino acids in the protein sequence. These isoforms and other naturally occurring isoforms are expressly encompassed by the term “receptor”. Naturally occurring or endogenous receptors can be recovered or isolated from a source which naturally produces the receptor, for example. These proteins and proteins having the same amino acid sequence as a naturally occurring or endogenous corresponding receptor, are referred to by the name of the corresponding mammal. For example, where the corresponding mammal is a human, the protein is designated as a human receptor.

As used herein, the term “ligand” refers to a compound that comprises at least one peptide, polypeptide, protein moiety that has a binding site with binding specificity for a desired target. For example, the ligand can comprise a (e.g., at least as one) protein moiety (e.g., a dAb) that has a binding site with-binding specificity for a receptor.

The terms “extracellular domain”, “ectodomain” or “ECD” are used interchangeably herein to refer to the extracellular region or a portion thereof exclusive of the transmembrane spanning and cytoplasmic regions. Ligand-binding ectodomains of the receptors described herein sense and bind target ligands including small molecules and proteins. The ectodomains of the receptors described herein may comprise homodimeric and heterodimeric ectodomain binding configurations. Homodimerization and heterodimerization may be ligand-induced.

As used herein, “transmembrane domain” or “TMD” broadly refers to an amino acid sequence of about 15 amino acid residues across the plasma membrane. More preferably, the transmembrane domain comprises at least about 20, 25, 30, 35, 40, or 45 amino acid residues and spans the plasma membrane. The transmembrane domain is abundant within hydrophobic residues and usually has an a-helical structure. In one embodiment, at least 50%, 60%, 70%, 80%, 90%, 95% or more of the amino acids in the transmembrane domain are hydrophobic, such as leucine, isoleucine, tyrosine, or tryptophan.

As used herein, “juxtamembrane domain” or “JMD” refers to an intracellular part of the receptor adjacent to the transmembrane domain.

In some embodiments, the extracellular domain of the human receptor protein is an extracellular domain of TGF-β receptor 1 (TGF-βR1) or TGF-β receptor 2 (TGF-βR2). In some embodiments, the extracellular domain of the human receptor protein is an extracellular domain of TNF receptor 1 (TNFR1) or TNF receptor 2 (TNFR2). In some embodiments, the extracellular domain of the human receptor protein is an extracellular domain of interleukin-10 receptor b (IL-10Rb) or interleukin-10 receptor a (IL-10Ra). In some embodiments, the extracellular domain of the human receptor protein is an extracellular domain of VEGF receptor 1 (VEGFR1) or VEGF receptor 2 (VEGFR2).

### II. MODULAR EXTRACELLULAR SENSOR ARCHITECTURE

The disclosed subject matter relates to integrated “Modular Extracellular Sensor Architecture” (MESA). In some embodiments, a MESA system includes a pair of extracellular receptors where both receptors of the pair contain a ligand binding domain and transmembrane domain, and one receptor contains a protease cleavage site and a functional domain (e.g., transcription regulator such as a transcription regulator that promotes transcription or a transcription regulator that inhibits transcription) and the other receptor contains a protease domain. In some embodiments, a MESA system includes a pair of extracellular receptors where both receptors of the pair contain a ligand binding domain and transmembrane domain, and one receptor contains half of a split protease domain and the other receptor contains the complementary half of the split protease domain as well as a protease cleavage site and a functional domain (e.g., transcription regulator such as a transcription regulator that promotes transcription or a transcription regulator that inhibits transcription). As used herein, a transcription regulator may include a transcription factor that promotes transcription (e.g., by recruiting additional cellular components for transcription) and/or a transcription inhibitor or transcription repressor).

MESA technology and the presently disclosed advancement may be utilized for building living cell-based biosensors. In certain embodiments, MESA technology and the presently disclosed advancement comprise engineered receptor proteins that can detect extracellular ligands (e.g., such as cytokines) and transduce this information across the cell membrane to release an engineered transcription regulator that drives the expression of a user-defined gene.

MESA technology and the presently disclosed advancement have a wide variety of uses including in vitro laboratory assays (e.g., to detect/quantify specific analytes), as powerful new experimental tools for studying in vivo animal models (e.g., wherein engineered cell-based biosensors could be adoptively transferred, generated from transplanted bone marrow, or genetically engineered in a transgenic animal), and as human therapeutics (e.g., for augmenting the functionality of engineered cell-based therapies). MESA technology and the presently disclosed advancement could also be adapted to function in other cell types, such as insect cells or microbes (e.g., yeast) to create cell-based biosensors for a variety of applications.

In certain embodiments of MESA technology and the presently disclosed advancement where two receptors are employed, the general mode of action is that ligand binding induces the aggregation of two or more MESA receptors, bringing an intracellular split protease domain (PR) into proximity with a complementary half of a split protease, leading to reconstitution of the protease and cleavage of the cognate intracellular protease cleavage site (PCS), and upon cleavage of the PCS by PR, a transcription factor (TF) or other functional domain (e.g., a transcription inhibitor) is released from the MESA receptor at the cell membrane to carry out its function (e.g., a TF may localize to the nucleus to induce gene expression). One implementation of this architecture would be a heterodimerization-(or heteromultimerization-) based signaling mechanism. In this system, one engineered receptor chain contains the N-terminal fragment of the split protease and the other engineered receptor chain contains the C-terminal fragment of the split protease as well as the PCS-TF domain. Other implementations include, for example, a system in which one receptor contains a complete protease and the other receptor contains the PCS-TF domain; or a homodimerization-(or homomultimerization-) based mechanism in which each MESA chain contains both PR and PCS-TF domains, but the receptor is engineered such that cleavage may occur in trans, but not in cis (i.e., one chain may not release its own TF).

A general implementation of one embodiment of MESA technology and the presently disclosed advancement is as follows: receptors are designed, DNA sequences encoding these receptors are generated (by molecular biology and/or DNA synthesis) and inserted into a suitable expression vector (such as a plasmid or a stable gene delivery system), the expression vector is transfected or genomically integrated into a suitable cell line or stock of primary cells (together with a suitable reporter construct, which expresses a reporter gene in response to nuclear-localized TF), ligand is added to the cell culture medium, and induced reporter gene expression is quantified by suitable means.

In certain embodiments, MESA technology and the presently disclosed advancement provide a cell-based biosensor for detecting a natural analyte of interest in vitro or in vivo and employing natural receptor ectodomains (ECDs) to coopt their mechanisms for ligand binding into customized transcriptional outputs.

In some embodiments, MESA technology and the presently disclosed advancement provide: i) an approach where a pair of MESA receptors are engineered with naturally occurring ligand-binding domains that recognize a specific peptide; ii) a cell-based biosensor for detecting a specific pattern of multiple analytes of interest (e.g., by coupling MESA receptors to engineered gene circuits to enable signal processing) in vitro; iii) a cell-based biosensor for detecting a specific pattern of multiple analytes of interest (e.g., by coupling MESA receptors to engineered gene circuits to enable signal processing) in vivo; iv) a cell-based biosensor coupled to expression of a gene that enables in vivo imaging (e.g., by MRI) for diagnostic purposes; v) a cell-based biosensor coupled to expression of a therapeutic agent to create targeted cellular therapies, which may be used to treat cancer, autoimmune disease, and other diseases; vi) a multicellular network using synthetic intercellular communication (e.g., engineering some cells to express MESA receptors and others to secrete MESA ligands), with applications including: scientific investigation of biological processes including development, immune function, wound healing, etc., cell & tissue-based products for applications including tissue engineering, regenerative medicine, immune therapy, transplantation medicine, cellular therapies and the like.

All MESA receptors can be modified in order to optimize specific receptor properties. Modifications include, for example, the following: i) varying the length of intracellular spacers (ISP) (on either MESA chain) to include, for example, between 0-20, 0-50, or 0-150 (e.g., 0 . . . 5 . . . 50 . . . 100 . . . 130 . . . or 150) non-structured amino-acid residues (e.g., glycines or alternating glycine-serine residues); ii) varying the predicted mechanical properties of ISP (on either MESA chain) by replacing non-structured amino acids with structured subdomains (e.g., an alpha-helical domain); iii) including an ESP domain (either structured or unstructured, of lengths between 0-20 or 0-50 or 0-150 amino acids). Structured domains may include, for example, an immunoglobulin motif, (e.g., for presentation of ligand-binding (LB) domains that are derived from antibody fragments at a certain distance away from the cell surface); altering the sequence of the PCS to enhance or inhibit the rate of PR-mediated cleavage; or varying the combinations of receptor chains used to constitute a complete MESA receptor system. The transmembrane domain may be derived from either natural or synthetic sequences in order to modulate the kinetics or geometry with which MESA chains associate in the presence or absence of ligand. The specific split TEVp domains on each receptor may also be modified. For example, either of both of the NTEVp and CTEVp domains may comprise mutations from the wildtype TEVp sequence that affect reconstitution propensities, and therefore signaling output from receptors may be modulated. See, Table 1 for wildtype and mutant split TEVp sequences.

MESA variants may use, for example, ligand-binding domain interactions including: i) using an antibody (or a fragment thereof) to bind to the target ligand; ii) implementation in a homodimeric MESA receptor (both antibody fragments are identical and bind to identical sites on a polyvalent ligand, such as a homodimeric cytokine); iii) implementation in a heterodimeric MESA receptor (e.g., each MESA chain incorporates a distinct antibody fragment, such that a monovalent ligand can still induce MESA receptor dimerization or multimerization); iv) incorporating a modular protein-peptide interaction that is not from a receptor ligand system (e.g., conserved protein motifs such as SH3, PDZ, and GBD domains bind distinct and unique consensus peptide motifs) to create an engineered MESA receptor-ligand system.

In certain embodiments, the released functional domain on a MESA receptor (ER-A) is replaced with another functional domain, such as a catalytic domain (whose activity requires cleavage-mediated release), a separate protease domain (whose activity requires cleavage-mediated release), a DNA-binding domain (e.g., zinc-finger or TAL Effector-based domains) coupled to a functional domain (e.g., an endonuclease, a chromatin modifying enzyme such as the Krueppel-associated box or KRAB protein, or other enzymes or cofactor-recruiting domains). In particular embodiments, modification of the MESA system to detect intracellular analytes, such that intracellular versions of the MESA receptors may be: ER-A could contain LB-ISP-PCS-TF domains and its cognate MESA receptor (ER-B) could contain LB-ISP-PR domains; in other embodiments ER-A could contain LB-ISP-PR1-PCS-TF domains and its cognate MESA receptor (ER-B) could contain LB-ISP-PR2 domains, where PR1 and PR2 are portions of a split protease. Ligand-binding by the two chains would again enable protease-mediated cleavage and release of a functional domain (such as transcription factor, TF). In other embodiments, the split protease-based MESA configuration may be modified to detect intracellular ligands.

MESA technology and the presently disclosed advancement may be configured for use in multiple cellular contexts for applications in basic science, biotechnology, and medicine (including both diagnostics and therapeutics). MESA biosensors and the disclosed improvements (e.g., implemented in mammalian cells) would have a wide variety of potential uses including in vitro laboratory assays (e.g., to detect/quantify specific analytes), as powerful new experimental tools for studying in vivo animal models (e.g., engineered cell-based biosensors could be adoptively transferred, generated from transplanted bone marrow, or genetically engineered in a transgenic animal to monitor extracellular species in real time in living animals), and potentially as human therapeutics (e.g., for engineering cell-based therapies that probe their environment and deliver a therapeutic payload only at desirable locations). This powerful synthetic biology technology may also be adapted to function in other cell types, such as insect cells or microbes (e.g., yeast) to create cell-based biosensors for applications in biotechnology.

In MESA technology and the presently disclosed advancement each engineered receptor (ER) of a receptor pair is composed of two chains, each of which is a type I transmembrane protein. The alpha chain (ER-A) may be fused at its C-terminus to one half of a split protease domain, a peptide harboring a TEV protease cleavage site (PCS), and an engineered transcription factor (TF). The beta chain (ER-B) may be fused at its C-terminus to the complementary half of the split TEV protease (PR). Other domains could include ligand-binding domains (LB), extracellular spacers (ESP), intracellular spacers (ISP) (e.g., which may be absent or a short length), transmembrane domains (TM), and juxtamembrane domains (JM). In such embodiments, the binding of ER-A and ER-B to a ligand may lead to receptor oligomerization, PR reconstitution, and PR-mediated cleavage and release of TF. This strategy is suitable for recognition of any ligand possessing more than one domain that may be recognized by a LB domain, as described in detail below. Modular receptor construction is intrinsic MESA technology and the presently disclosed advancement, since receptor design may, in certain embodiments, require adjustment for each receptor-ligand combination. Domain junctions may be engineered by introducing unique restriction sites to facilitate exchange.

MESA technology and the presently disclosed advancement may rely upon the formation of heteromeric complexes. In alternative embodiments, each chain may include both PR and TF domains separated by a PCS and oriented such that each PR domain cleaves in trans but not in cis (i.e., PR cleaves neighboring receptors upon ligand binding-induced aggregation).

In some embodiments, the presently disclosed systems provide cell-based biosensors that perform multifactorial logical evaluation of extracellular signals using the MESA receptors described herein, which transduce extracellular cues into synthetic pathways. Such pathways may be constructed into genetic circuit architectures that can process information in useful ways. For example, one may engineer cells to perform multifactorial evaluations of extracellular inputs using Boolean logic, which is a strategy that has been implemented to date using intracellular sensors. Successful implementation of this strategy using extracellular inputs is an important step toward building mammalian cell-based sensors that interface with natural systems in vivo. For example, initially, three representative types of circuits may be constructed such as “OR”, “NOT IF”, and “AND” gate genetic circuits. Transcriptional control may be implemented using systems known in the art and described herein.

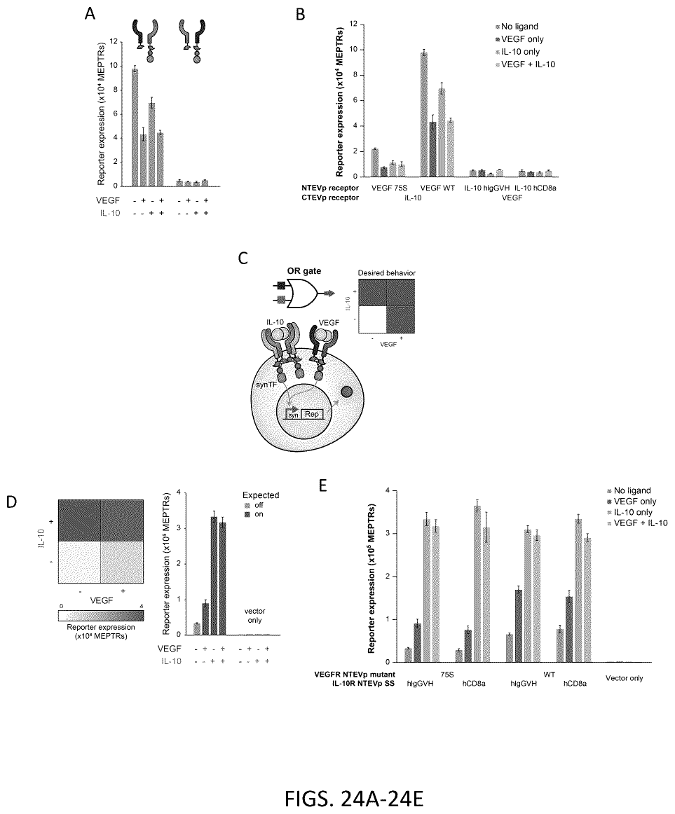

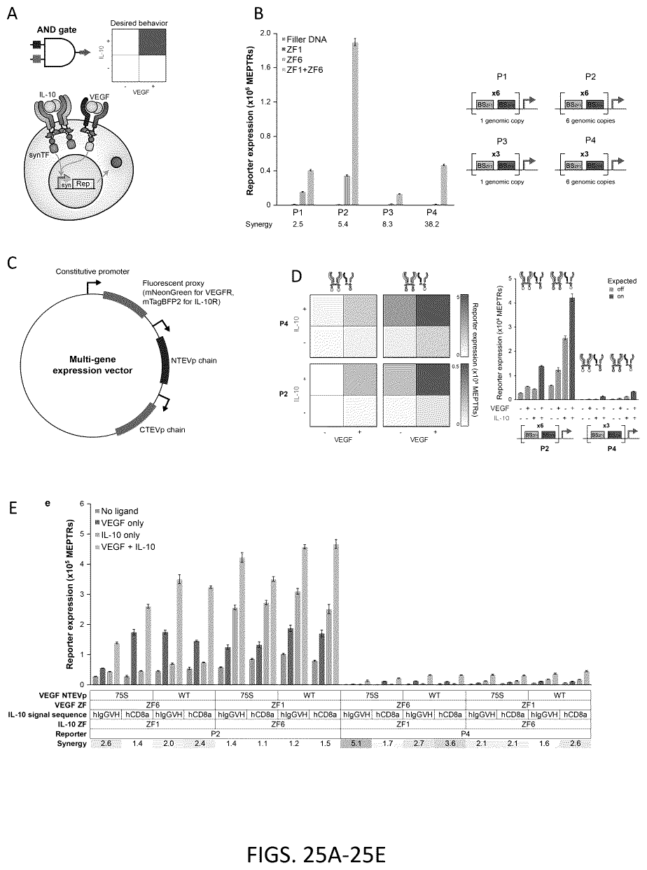

In particular, one may investigate the following circuit architectures, each of which would be useful for probing immune function: an OR gate that reports in response to either IL-10 or VEGF (i.e., a general sensor for immunosuppressive signals) as shown in FIG. 24C for example, a NOT IF gate that reports in response to IL-10 but only when IL-12 is absent (i.e., a sensor for uniformly immunosuppressive signals), and an AND gate that reports only in response to IL-10 and VEGF (i.e., a sensor specific for multimodal immunosuppressive signals) as shown in FIG. 25A for example. After characterizing the qualitative behavior of these circuits, one may also characterize the quantitative function of these circuits (see, Example 6). These investigations may be facilitated by choice of engineered transcription factors. Plasmid doses may also be varied to modulate the level of engineered receptor expression. Using these tunable parameters, one may determine the sensitivity of these circuits to various input combinations and strengths (concentrations) and characterize the resulting transfer functions (quantitative relationships between inputs and outputs). In some embodiments, logic gates having multiple inputs may be generated where one input is a ligand as described herein, and the other input is a physiological state (e.g., hypoxia) that effects a response. For example, where the input is the physiological state of hypoxia, a hypoxia-responsive protein/promoter may be used to regulate part of the signaling downstream of MESA, such that the output gene is expressed only under conditions of hypoxia AND in the presence of the ligand for the MESA receptor.

One may also evaluate the dynamic responses of these circuits when extracellular inputs are removed from the system (e.g., by replacing the culture medium). To facilitate these analyses, one may use computational mathematical modeling, as has previously been done for other intracellular genetic circuits. An important extension may be developing systems for stably expressing these circuits and characterizing their performance under these expression conditions. One may need to evaluate the influence of expression on circuit performance, stability, and variability. Strategies for coping with these challenges include expressing both receptor chains (ER-A and ER-B) from a single multicistronic vector, which reduces the number of vectors required. Bicistronic expression would suffice for implementing even the relatively more complicated “AND” gate.

In certain embodiments, MESA technology and the presently disclosed advancement described herein are implemented in mammalian cells, and are employed in any suitable use, such as in vitro laboratory assays (e.g., to detect/quantify specific analytes), as powerful experimental tools for studying in vivo animal models (e.g., engineered cell-based biosensors could be adoptively transferred, generated from transplanted bone marrow, or genetically engineered in a transgenic animal to monitor extracellular species in real time in living animals), and as human therapeutics (e.g., for engineering cell-based therapies that probe their environment and delivery a therapeutic payload only at desirable locations). In other embodiments, MESA technology and the presently disclosed advancement are employed with other cell types, such as insect cells or microbes (e.g., yeast) to create cell-based biosensors for applications in biotechnology.

Any type of suitable ligand binding domain (LB) can be employed with the receptors of MESA technology. Ligand binding domains can, for example, be derived from either an existing receptor ligand-binding domain or from an engineered ligand binding domain.

Existing ligand-binding domains could come, for example, from cytokine receptors, chemokine receptors, innate immune receptors (TLRs, etc.), olfactory receptors, steroid and hormone receptors, growth factor receptors, mutant receptors that occur in cancer, neurotransmitter receptors. Engineered ligand-binding domains can be, for example, single-chain antibodies (see scFv constructs discussion below), engineered fibronectin based binding proteins, and engineered consensus-derived binding proteins (e.g., based upon leucine-rich repeats or ankyrin-rich repeats, such as DARPins). The presently disclosed advancement utilizes naturally occurring ligand-binding domains to coopt their mechanisms for ligand binding into customized transcriptional outputs.

Any suitable extracellular spacer (ESP) can be used with the receptors of MESA technology and the presently disclosed advancement. In certain embodiments, the ESP is from 0-30 amino acids long (e.g., 1 . . . 5 . . . 15 . . . 25 . . . or 30), where each amino acid can be, for example, any of the 20 naturally occurring amino acids. In certain embodiments, ESP can be nonstructured or comprised partially or entirely of amino acids predicted to fold into a secondary structure (i.e., an alpha helix) or a tertiary structure. ESP sequences flanking the transmembrane (TM) domain may be selected to adjust the stability of the TM in the membrane (i.e., adding a polar or charged residue to ESP next to TM should make it more difficult for that amino acid to be pulled into the membrane). In certain embodiments, ESP is derived from the extracellular portion of a natural receptor sequence.

Any suitable transmembrane domain (TM) can be used with the receptors of MESA technology and the presently disclosed advancement. In certain embodiments, the TM is, for example, a TM domain taken from an existing receptor (e.g., TLR4, CD28, IL-10 receptor, VEGF receptor, TGF-β receptor, TNF receptor etc.) or engineered using a novel sequence, for example using TM consensus sequence features.

Any suitable intracellular spacer (ISP) can be used with the receptors of MESA technology and the presently disclosed advancement. In particular embodiments, no ISP is present. In certain embodiments, the ISP is, for example, 0-30 amino acids long (e.g., 1, 2, 3, 4, 5, 6, . . . 15 . . . 25 . . . or 30 amino acids) where each amino acid can be, for example, any of the 20 naturally occurring amino acids. ISP can be, for example, nonstructured or comprised partially or entirely of amino acids predicted to fold into a secondary structure (i.e., an alpha helix) or a tertiary structure. ISP sequences flanking the TM domain may be selected to adjust the stability of the TM in the membrane (i.e., adding a polar or charged residue to ISP next to TM should make it more difficult for that amino acid to be pulled into the membrane). In certain embodiments, ISP is derived from the intracellular portion of a natural receptor sequence.

Any suitable protease cleavage sequence may be employed with the receptors of MESA technology and the presently disclosed advancement. In certain embodiments, the PCSs, for example, are varied by mutating the amino acid at the P1′ position, for example, to any of the 20 amino acids or by introducing 1 or more mutations into the rest of the PCS, e.g., to modify kinetic parameters governing PCS cleavage.

MESA technology and the presently disclosed advancement are not limited to any particular protease or corresponding protease cleavage site. In some embodiments, the protease and cleavage site are from a virus. For example, in certain embodiments, the protease and protease cleavage site are from a virus selected from: tobacco etch virus (TEV), a chymotrypsin-like serine protease and corresponding cleavage sites, alphavirus proteases and cleavage sites, Hepatitis C virus proteases (e.g., N S3 proteases) and corresponding cleavage sites, chymotrypsin-like cysteine proteases and corresponding cleavage sites, papain-like cysteine proteases and cleavage sites, picornavirus leader proteases and cleavage sites, HIV proteases and cleavage sites, Herpesvirus proteases and cleavage sites, and adenovirus proteases and cleavage sites (see, Tong, Chem. Rev. 2002, 102, 4609-4626, herein incorporated by reference in its entirety). In particular embodiments, the proteases and cleavage sites are bacterial in original, such as, for example, from Streptomyces griseus protease A (SGPA), SGPB, and alpha-lytic protease and corresponding cleavage sites. In some embodiments, the proteases and cleavage sites are mammalian. For example, the proteases could be one of the five major classes of proteases known in mammals which include serine proteases, cycteine proteases, metallo proteases, aspartic proteases, and thereonine proteases (see, e.g., Turk et al., The EMBO Journal, 2012, 31, 1630-1643; Lopez-Otin and Overall, 2002, Nat. Rev. Mol. Cell Biol., 2:509-519; Overall and Blobel, 2007, Nat. Rev. Mol. Cell Biol., 8:245-257; and Lopez-Otin and Bond, 2008, J. Biol. Chem., 283:30422-30437, all of which are herein incorporated in their entireties by references.

In certain embodiments, receptors for MESA technology and the presently disclosed advancement may be designed using engineered ligand binding domains based upon single chain antibody variable fragments (scFv). The loop linking heavy and light chain-derived fragments of an scFv may be designed (both in length and sequence) to favor monomeric scFvs, dimeric scFvs, trimeric scFvs, etc. Loop length may be, for example, 0-30 amino acids long, where each amino acid may be, for example, any of the 20 naturally occurring amino acids. One may select a loop to favor scFvs or to favor homomultimeric scFvs. ScFv may be engineered, for example, from isolated antibody, BCR, or TCR sequences, or they may be isolated from a random library, such as phage-display, bacterial-display, or yeast-display. In other embodiments, receptors for MESA technology and the presently disclosed advancement may be designed using engineered ligand binding domains based upon a camelid antibody analog termed a “nanobody.”

In certain embodiments, directed evolution could be used to optimize performance characteristics of receptors including, for example: low background signaling, enhanced signal-to-noise ratio, enhanced sensitivity for low ligand concentrations, and enhanced dynamic range (differential responsiveness over a wider range of ligand concentrations). Directed evolution could be performed, for example, by a scheme in which (a) a library of genetic variants upon an initial receptor design are created (b) each variant is expressed in a separate cell (c) this pool of cells is exposed to a functional screen to either eliminate cells (and therefore receptor variants) exhibiting undesirable activity or retain cells (and therefore receptor variants) that exhibit some desirable activity. This process could be repeated to enrich for variants with desirable properties. A variation upon this method would be to isolate variants in this fashion after 1 or more rounds of enrichment, introduce additional genetic diversity into this library, and return to the cell-based screening; this could be repeated for multiple rounds until the pool or individual constructs within the pool exhibit properties that meet some threshold for considering it a success. In some embodiments, one could (a) generate a library of DNA sequences encoding MESA variants using error-prone PCR or other molecular biology techniques to incorporate chemically synthesized DNA oligonucleotides including variation at defined positions; variation could be introduced at ISP, PCS, PR, TM, ESP, LB, or combinations of these sites, (b) each variant could be cloned into an expression vector based upon adeno-associated virus (AAV), viral vectors could be packaged by standard techniques, and AAV vectors could be used to transduce cells at a ratio of viruses to cells such that each cell expresses only one variant of the MESA library, and then (c) this pool of cells that expresses the MESA library (one variant per cell) could be used for cell-based assays; for example, cells could be transfected or transduced with a reporter construct that reads out MESA signaling by inducing expression of a fluorescent protein, and then the MESA pool of reporter-bearing cells could be sorted using fluorescence assisted cell sorting (FACS) based upon whether the reporter construct is induced or not when exposed to zero ligand or some finite quantity of ligand.

### III. NATURAL ECTODOMAIN MESA SYSTEM (NATE MESA)

To build receptors that sense soluble, physiological cues and produce user-defined transcriptional output in response, the Modular Extracellular Sensor Architecture (MESA) was used (FIG. 1A, Table 1) (Daringer, et al. ACS Synthetic Biology, 2014; Dolberg, et al. Nature Chemical Biology, 2021). MESA receptors comprise two types of transmembrane proteins that are engineered to associate upon ligand binding and release a sequestered transcription factor. Both MESA receptor types contain a signal peptide, a 3×FLAG epitope tag, extracellular domains that bind to the target ligand, and a transmembrane domain. Inside the cell membrane, one type contains the C-terminal half of a split tobacco etch virus (TEV) protease, a protease recognition sequence, and an intracellular transcription factor. The second type contains the N-terminal half of the split TEV protease inside the cell. Upon extracellular ligand binding, the two receptor types are driven to reconstitute the intracellular split protease and release the tethered transcription factor to translocate to the nucleus and activate target gene expression (FIG. 1A). Published versions of MESA receptors contain small molecule binding domains, scFvs, and nanobodies as extracellular ligand binding domains (Edelstein, et al. Synthetic Biology, 2020; Dolberg, et al. Nature Chemical Biology, 2021). In embodiments described herein, natural receptor ectodomains were identified, which may also be used to confer MESA receptor binding to their cognate ligands, with the specific construction dependent on the biophysical ligand-binding mechanism of the natural parental receptor system. Wildtype and mutant MESA sequences can be found in Table 1.

TABLE 1
Sequences of the intracellular binding domain

| Name | SEQ ID NO | Sequence |
|---|---|---|
| NTEVp wildtype | 1 | ESLFKGPRDYNPISSTICHLTNESDGHTTSLYGIG |
| | | FGPFIITNKHLFRRNNGTLLVQSLHGVFKVKNT |
| | | TTLQQHLIDGRDMIIIRMPKDFPPFPQKLKFREP |
| | | QREERICLVTTNFQT |
| CTEVp wildtype | 2 | KSMSSMVSDTSCTFPSSDGIFWKHWIQTKDGQ |
| | | CGSPLVSTRDGFIVGIHSASNFTNTNNYFTSVPK |
| | | NFMELLTNQEAQQWVSGWRLNADSVLWGGH |
| | | KVFMVKPEEPFQPVKEATQLMN |
| NTEVp 75S | 3 | ESLFKGPRDYNPISSTICHLTNESDGHTTSLYGIG |
| | | FGPFIITNKHLFRRNNGTLLVQSLHGVFKVKNT |
| | | TTLQQSLIDGRDMIIIRMPKDFPPFPQKLKFREP |
| | | QREERICLVTTNFQT |
| CTEVp 190K | 4 | KSMSSMVSDTSCTFPSSDGIFWKHWIQTKDGQ |
| | | CGSPLVSTRDGFIVGIHSASNFTNTNNYFTSVPK |
| | | NFMELKTNQEAQQWVSGWRLNADSVLWGGH |
| | | KVFMVKPEEPFQPVKEATQLMN |
| NTEVp 103H | 5 | ESLFKGPRDYNPISSTICHLTNESDGHTTSLYGIG |
| | | FGPFIITNKHLFRRNNGTLLVQSLHGVFKVKNT |
| | | TTLQQHLIDGRDMIIIRMPKDFPPFPQKLKFREH |
| | | QREERICLVTTNFQT |
| NTEVp 75E | 6 | ESLFKGPRDYNPISSTICHLTNESDGHTTSLYGIG |
| | | FGPFIITNKHLFRRNNGTLLVQSLHGVFKVKNT |
| | | TTLQQELIDGRDMIIIRMPKDFPPFPQKLKFREP |
| | | QREERICLVTTNFQT |
| CTEVp 158P | 7 | KSMSSMVSDTSCTFPSSDGIFWKHWIQTKDGQ |
| | | CGSPLVSPRDGFIVGIHSASNFTNTNNYFTSVPK |
| | | NFMELLTNQEAQQWVSGWRLNADSVLWGGH |
| | | KVFMVKPEEPFQPVKEATQLMN |
| Protease | 8 | ENLYFQM |
| cleavage site | | |
| (PCS) | | |
| Functional | 9 | PKKKRKVSGDALDDFDLDMLGSDALDDFDLD |
| domain | | MLGSDALDDFDLDMLGSDALDDFDLDMLGSG |
| | | GGGSGGGGSGGGGSGTARPGERPFQCRICMRN |
| | | FSKGERLVRHTRTHTGEKPFQCRICMRNFSRMD |
| | | NLSTHLRTHTGEKPFQCRICMRNFSRKDALNRH |
| | | LKTHLRGS |

To first convert natural receptors into NatE MESA receptors, domains were pulled directly from natural human receptors and employed into the MESA framework, including the signal peptide, complete extracellular domain, transmembrane domain, and 5-12 amino acids of the juxtamembrane domain (FIG. 1B). Variants that contained alternative natural signal peptides (derived from the human CD8a receptor and the human IgG variable heavy chain), alternative transmembrane domains (derived from the murine or human CD28 receptor), and alternative juxtamembrane domains (synthetic, flexible sequences comprised of glycine and serine amino acids) were also tested. Receptor panels were constructed of native human receptors that represent a variety of receptor classes and signaling mechanisms and that could enable useful biosensing capabilities for soluble disease markers. These receptors included human VEGFR1 and VEGFR2 (for sensing vascular endothelial growth factor, VEGF), IL-10Ra and IL-10RB (for sensing interleukin 10, IL-10), TNFR1 and TNFR2 (for sensing tumor necrosis factor, TNF), and TGF-βR1 and TGF-βR2 (for sensing transforming growth factor B, TGF-β). This panel was selected because VEGF, IL-10, and TGF-B are upregulated soluble cues in the tumor microenvironment and TNF and IL-10 are important immunoregulatory molecules that play roles in inflammatory and immunosuppressive states respectively. (FIG. 1C).

The receptor systems chosen included hetero-associative (different receptor chains associate) and homo-associative (like receptor chains associate) ligand-dependent signaling mechanisms between two or more receptor chains, making them promising candidates for use in a ligand-mediated split protease reconstitution and trans-cleavage (MESA-like) signaling mechanism.

Some NatE MESA variants additionally employ a signal peptide, transmembrane domain, and juxtamembrane domain from a parent natural receptor for the target ligand to signal inducibly. Some NatE MESA variants utilize (or see performance improvements with) alternative natural or synthetic signal peptides, transmembrane domains, and juxtamembrane domains. Some NatE MESA variants may utilize mechanism-guided tuning of the intracellular signaling domains to signal inducibly. Finally, some NatE MESA variants are unable to signal inducibly, highlighting the non-obvious nature of this conversion.

The present disclosure provides novel synthetic receptors that enable mammalian cell-based biosensing of physiological ligands that outperform existing synthetic receptors. This disclosure also establishes a workflow and design principles for the generation of additional synthetic receptors for other targets. These synthetic receptors enable controlled gene expression (i.e. therapeutic secreted protein or other downstream gene circuitry) in response to detection of extracellular ligands in cell-based devices.

Prior to the present disclosure, no one has tried to systematically convert multiple types of natural cytokine receptors into synthetic sensors that produce custom transcriptional output, as described here. One VEGFR ectodomain-based synthetic receptor system exists, though this system has not demonstrated inducible signaling in response to recombinant, extracellular ligand (Baeumler, et al.). Others have employed well-characterized protein-protein interactions downstream of native GPCR signaling (Barnea, et al., Kipniss, et al., Baeumler, et al.) and ErbB signaling (Chung, et al.) to convert ligand binding into custom transcriptional output.

The disclosed sensors and systems (i.e., NatE MESA) can be utilized for, among other thing, cell-based therapies in which the sensor detects a disease marker of interest and response by regulating production of a therapeutic output; cell-based; diagnostics/theragnostics to report on presence or relative abundance of a disease marker of interest by regulating production of a reporter gene; and products enabling fundamental research on the dynamics of selected disease markers through the course of disease or in in vitro laboratory assays to detect and quantify target ligands.

Unlike the natural receptors from which these synthetic sensors are derived, the disclosed sensors signal through orthogonal signaling pathways that are self-contained and have the advantage of minimally disturbing or being regulated by native cellular processes. In particular, these sensors can signal via transcription factors of the Composable Mammalian Elements of Transcription (COMET), a panel of synthetic zinc finger-based transcription factors, which makes them easier to use with sophisticated downstream circuitry. These receptors are also amenable to other user-defined synTFs besides those from the COMET toolkit.

The disclosed sensors demonstrate substantially higher surface expression, leading to improved ability to sense ligands outside of the cell compared to previously-reported sensors. The strategy employed here spans three classes of receptors (cytokine receptors, receptor tyrosine kinases, receptor serine/threonine kinases) and identifies the most important aspects of design of synthetic receptors from natural receptors to propose generalizeable rules for this process.

In sum, the present disclosure provides cell-based biosensing capabilities to engineer cell-based devices to sense and respond to physiological ligand targets. In particular, the present disclosure includes engineering custom transcriptional output upon detection of tumor microenvironment cues (VEGF, IL-10, TGF-β), detection of general inflammation (TNF), and detection of general immunosuppression (IL-10). Further, these characterizations have identified general properties of natural receptors that enable conversion into synthetic modular receptors and will enable the development of more biosensors. Accordingly, this technology has value for many types of cell-based therapies, diagnostics, and research tools.

### IV. VASCULAR ENDOTHELIAL GROWTH FACTOR (VEGF)

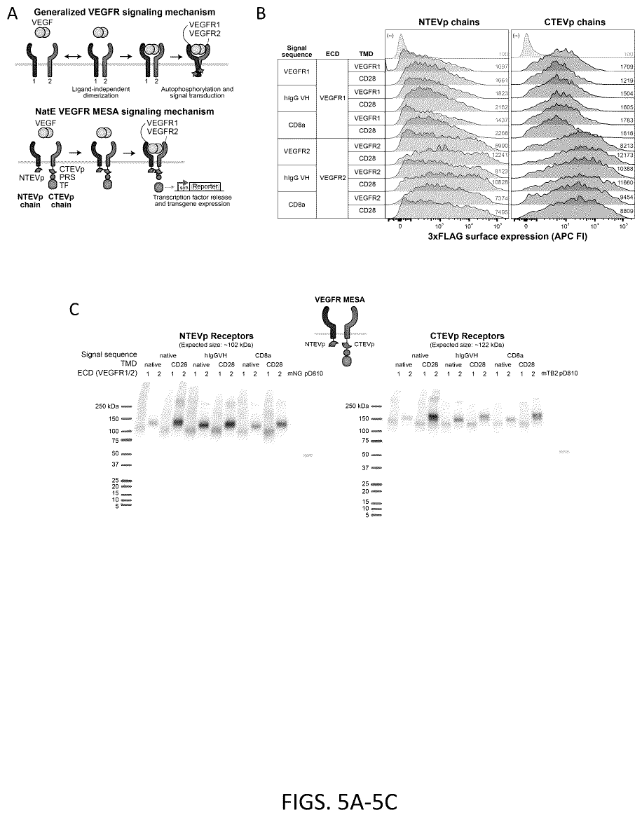

Vascular endothelial growth factor (VEGF) performs a key role in neovascularization, and its dysregulated activity leads to solid tumor growth and development. This molecule is thus upregulated in many tumor tissues and has been harnessed as a tumor microenvironment (TME) marker and therapeutic target. VEGFR1 and 2 belong to the receptor tyrosine kinase (RTK) family, and are the main receptors involved in VEGF signaling and angiogenesis. These receptors can homo- or heterodimerize in the absence of VEGF, but do not signal without VEGF. All VEGF isoforms exist mainly as homodimers. Ligand binding to the extracellular domain induces a conformational change, which leads to the activation and autophosphorylation of the intracellular kinase domains, activating different signaling cascades downstream (FIG. 5A). Given previous studies that elucidated the MESA receptor signaling mechanism and proof of principle demonstrations with VEGFR domains and a similar signaling mechanism, it was hypothesized that this ligand-induced conformational change could be compatible with MESA's mutant split TEVp reconstitution and cleavage mechanism, resulting in inducible synthetic biosensors that sense VEGF.

### V. INTERLEUKIN-10 (IL-10)

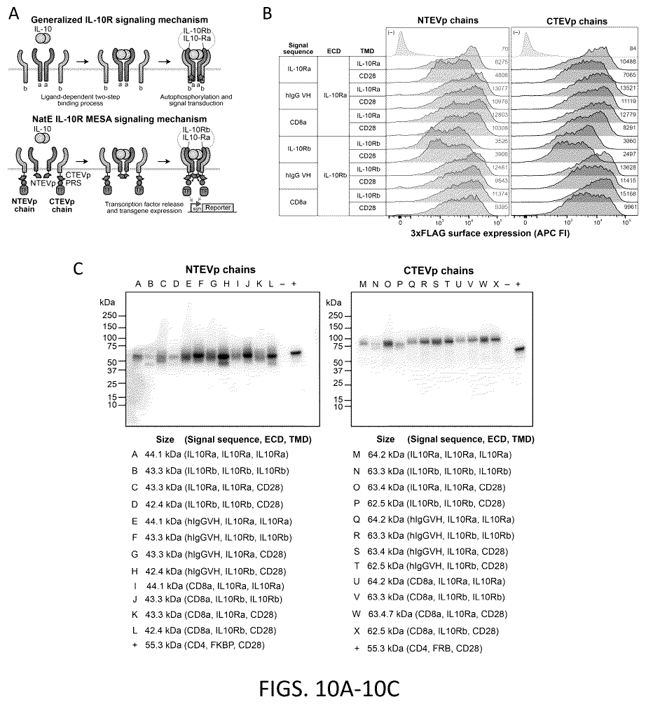

Interleukin-10 (IL-10) is an immunoregulatory cytokine, and its dysregulation is implicated in many diseases. Overexpression of IL-10 can suppress immune responses to pathogens or other threats while down-regulation of IL-10 can mediate chronic inflammation. Additionally, many types of cancer are associated with high local concentrations of IL-10 in the TME, including melanoma, ovarian cancer, and several lymphomas. Targeting IL-10 with a cell-based therapy may permit targeting immunostimulative therapies to the tumor specifically. IL-10 is a homodimeric ligand that signals through IL-10Ra and IL-10RB, which are considered class II cytokine receptors. Signaling occurs via a mechanism by which the high-affinity receptor pair IL-10Ra binds to the ligand first, eliciting a conformational change that permits the low-affinity receptor pair IL-10RB to bind and form the full heterotetrameric complex which signals through the JAK/STAT signaling pathway (FIG. 10A). Notably, signaling is still permitted with mutant forms of IL-10 that are monomeric suggesting that some signaling occurs within single a and B chains bound to a monomer of the ligand as opposed to only across the dimer. Some studies suggest that IL-10Ra/β complexes may be weakly pre-assembled to some extent, and other studies confirm that ligand induces an increase in homo- and hetero-association of the two receptor types. Because of these mechanistic features, it was hypothesized that IL-10 could induce association of MESA receptors IL-10R ECDs and resulting split TEVp reconstitution and cleavage.

### VI. TRANSFORMING GROWTH FACTOR β (TGF-β)

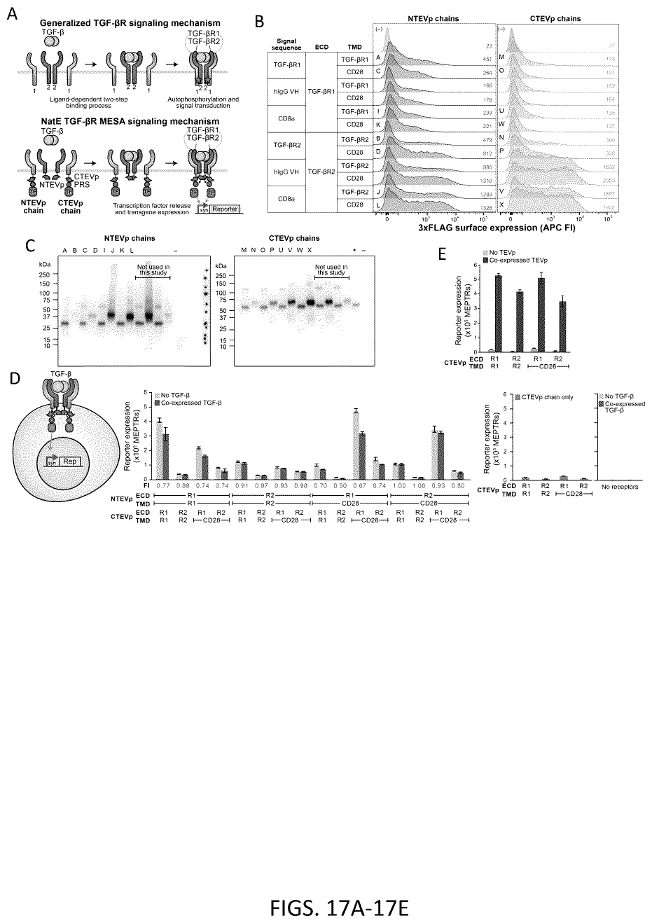

Transforming growth factor β (TGF-β) is a multifunctional cytokine that has multiple (sometimes opposing) functions depending on its context. TGF-β generally plays a highly immunosuppressive role, downregulating effector functions and expansion of many immune cell types. These properties also make TGF-β a tumor suppressor, halting growth of pre-malignant cells. Throughout cancer development, however, TGF-β supports metastasis by suppressing immune surveillance and cytotoxic activity in the tumor microenvironment. As a result, targeting TGF-B with a cell-based therapy that drives customized transcriptional output could enable tumor targeting of cytotoxic functions in a surface marker-independent manner. TGF-β, in its mature form, is a homodimeric ligand that signals through TGF-βR1 and TGF-βR2, which are serine/threonine kinase receptors that drive SMAD signaling pathways. A TGF-βR2 pair first binds to TGF-β, and then TGF-βR1 is recruited to form a heterotetrameric complex (FIG. 17A). Both receptor types are generally found as monomers in the absence of ligand, but TGF-βR1 can be found partially pre-dimerized when overexpressed. The monomeric nature of these receptors in the absence of ligand and the ligand-mediated heteroassociation are both attractive features for conversion into a ligand-induced split TEVp-reconstitution-based mechanism.

### VII. TUMOR NECROSIS FACTOR (TNF)

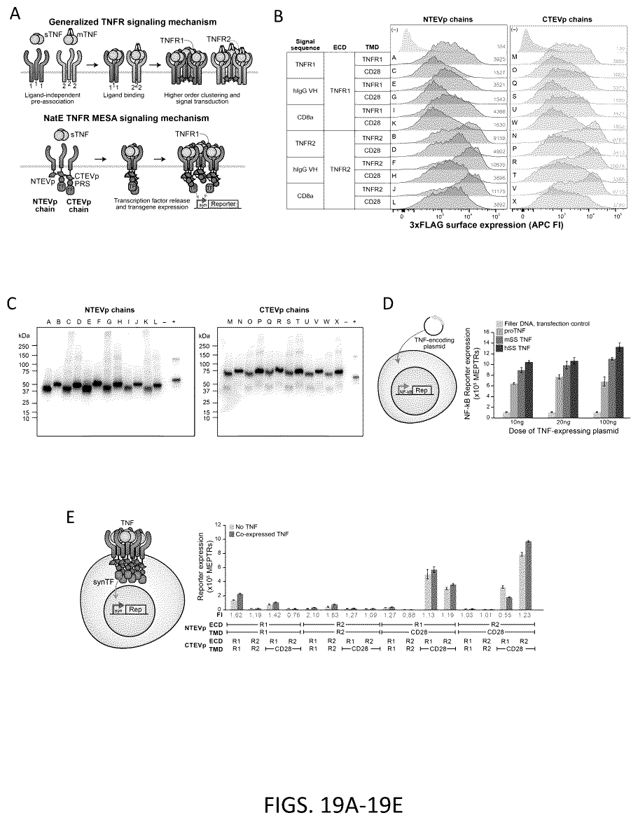

Tumor necrosis factor (TNF) is an immunostimulatory cytokine that is involved in inflammatory responses and highly upregulated locally and systemically in most chronic inflammatory diseases. Targeting TNF with a cell-based therapy could permit sensing and responding to local sites of inflammation with immunosuppression, which could help manage chronic inflammatory disease without systemically immunosuppressing the patient and increasing their risk of infection and malignancy. TNF is a homotrimeric ligand that signals through TNFR1 or TNFR2, which are part of the TNF cytokine receptor class, and each activate different signaling pathways. TNF can be found in the human body in membrane-tethered form (mTNF) or soluble form (sTNF). TNFR1 has higher affinity for TNF and can sense either mTNF or STNF, while TNFR2 has lower affinity and mainly senses mTNF with only weak interactions with sTNF. Both receptors have similar mechanisms of signaling-TNFRs pre-associate in the absence of ligand with each other in a homo-associative manner as these associations are mediated by various domains in the ECD itself and the stalk regions. Ligand binding induces a conformational change in these complexes as well as higher order homo-associations with other receptor complexes to transduce signaling (FIG. 19A). It was hypothesized that this high order clustering mechanism might be amenable to split TEVp reconstitution when receptors with matching ECDs are paired. It was also hypothesized that we could take advantage of smaller clusters of pre-associated receptors with the same ECD to sequester the MESA signaling components separately and drive heteroassociation upon ligand addition.

### VIII. EXAMPLES

### Materials and Methods

General DNA assembly: Plasmid cloning was performed primarily using standard PCR and restriction enzyme cloning with Phusion DNA Polymerase (NEB #M0530L), restriction enzymes (NEB), T4 DNA Ligase (NEB #M0202L), Antarctic Phosphatase (NEB #M0289L) and T4 Polynucleotide Kinase (NEB M0201L). Golden gate assembly and Gibson assembly were also utilized. Plasmids with pcDNA-based backbones (including transcription unit positioning vectors, TUPVs) were transformed into chemically competent TOP10 Escherichia coli (Thermo Fisher #C404010), and cells were grown at 37° C. Plasmids with poly-transfection, transposon, lentiviral, integration vector backbones were transformed into chemically competent NEB Stable Escherichia coli (NEB #C3040H), and cells were grown at 30° C.

Source vectors for DNA assembly: Genes encoding each ligand were sourced from: VEGFA165 (pVax1-hVEGF165, Addgene plasmid #74466), IL-10 (pHR_Gal4UAS_humanIL-10_T2A_PDL1_PGK_mCherry, Addgene plasmid #85430), TNF (pLI_TNF, Addgene plasmid #171179), and TGF-β1 (TGFB1_pLX307, Addgene plasmid #98377). DsRed-Express2 was obtained by site directed mutagenesis of pDsRed2-N1. The red crt operon used in mMoClo destination vectors was sourced from the landing pad destination vector plasmid. The cHS4 insulator was sourced from PhiC31-Neo-ins-5xTetO-pEF-H2B-Citrin-ins (Addgene plasmid #78099). The CAG promoter was sourced from pR26R CAG/GFP Asc, (Addgene plasmid #74285). The human EFla promoter was sourced from pLVX-Tet3G (Clontech/Takara). BlastR was sourced from lenti dCAS-VP64 Blast, (Addgene plasmid #61425). PuroR and HygroR were sourced from pGIPZ (Open Biosystems). Genes encoding natural transcription factors were sourced from: cJun (pCLXSN-c-JUN, Addgene plasmid #102758) and BATF (pFUW-TetO-BATF, Addgene plasmid #178451). The NF-κB inducible promoter was sourced from pLNEE. PiggyBac transposon inverted terminal repeats were sourced from pPB_Muc1_mOxGFP_dCT_BlpI. Sleeping Beauty transposon inverted terminal repeats were sourced from pT4/HB (Addgene plasmid #108352). The gene encoding hyPBase, a hyperactive from of the PiggyBac transposase for high efficiency insertion, was sourced from pCMV-hyPBase. The gene encoding SB100X, a hyperactive form of the Sleeping Beauty transposase for high efficiency insertion, was sourced from pCMV (CAT) T7-SB100, (Addgene plasmid #34879).

Cloning MESA receptors: In most cases, MESA receptors were first cloned into poly-transfection backbones, which are modified versions of pcDNA to confer high expression in HEK293FT cells and include a secondary transcriptional unit encoding constitutive expression of a fluorescent protein. These plasmid backbones are derived from a modified version of the pcDNA3.1/Hygro (+) Mammalian Expression Vector (Thermo Fisher #V87020), (pPD005, Addgene #138749) and further modified to change the antibiotic resistance marker from ampicillin to kanamycin and to add the additional expression cassette containing a CMV promoter, fluorescent protein gene, and polyadenylation sequence. Receptors with the CTEVp domain were cloned into a poly-transfection backbone with a constitutively expressed mTagBFP2 gene, while receptors with the NTEVp domain were cloned into a poly-transfection backbone with a constitutively expressed mNeonGreen gene. In general, restriction sites were chosen to facilitate modular swapping of parts via restriction enzyme cloning. Selected receptor constructs were moved into TUPV backbones by restriction enzyme-based cloning to facilitate transposon vector assembly via mammalian modular cloning (mMoClo). In some cases, receptors were cloned directly into TUPV backbones.

Cloning ligands for co-expression and secretion: Ligands were cloned into a pcDNA backbone to confer high expression in HEK293FT cells (Addgene #138749). PCR was used to amplify the coding region of each ligand and append a secretion signal sequence if necessary and restriction enzyme-based cloning was used to insert the products into pcDNA. Selected ligands were moved into a second generation pGIPZ lentiviral vector by restriction enzyme-based cloning to enable generation of stable ligand-secreting HEK293FT and SKOV3 cell lines.

Cloning reporters: Golden Gate assembly was used to construct all synthetic TF-responsive reporters in a TUPV backbone to facilitate assembly into transposon or landing pad integration vectors. The specific TUPV backbone used for golden gate reporter assembly (pGGB022) includes a pair of BsaI restriction sites upstream of a YB_TATA minimal promoter and a DsRedExpress-2 reporter gene. Promoter inserts containing TF binding sites were synthesized as 15-100 bp oligonucleotides (some promoters were long enough to require multiple inserts) by Integrated DNA Technologies or Life Technologies (Thermo Fisher). The coding and reverse strands were synthesized separately and designed to anneal, resulting in dsDNA with a 4 nt sticky end overhang on each side. The coding and reverse oligonucleotides were mixed (6 μL H₂O, 1 μL T4 Ligase Buffer, 1 μL T4 PNK (10 U/μL; NEB), 1 μL of each 100 μM oligonucleotide) and phosphorylated at 37° C. for 1 h. They were then denatured at 95° C. for 5 min and cooled slowly to room temperature (approximately 22° C.) to allow for annealing. The mix was then diluted 500-fold to make a 20 nM stock and included in the Golden Gate reaction. Golden Gate reaction mixtures comprise 1 μL T4 ligase buffer, 1 μL 10×BSA (1 mg/mL), 0.5 L BsaI-HF (20 U/μL; NEB), 0.5 μL T4 Ligase (400 U/μL; NEB), 10 fmol of vector, 1 μL of each insert (diluted to 20 nM), and water to a total volume of 10 μL. The reaction was incubated at 37° C. for 1 h, 55° C. for 15 min, and 80° C. for 20 min, and then cooled to room temperature. Then, 3 μL of the reaction was immediately transformed into 50 μL of chemically competent TOP10 E. coli.

Natural TF-responsive promoters were constructed using restriction enzyme-based assembly. Promoters were synthesized using the same oligo annealing approach described for synthetic TF-responsive promoters. These inserts were diluted 1:500 to generate a 20 nM stock. The backbone used was a TUPV plasmid containing a different array of binding sites, a YB_TATA minimal promoter, and a DsRedExpress-2 reporter gene. The backbone was cut upstream of the binding site array using BglII (NEB) and in the YB_TATA minimal promoter using SpeI-HF (NEB). The natural TF binding site array inserts were designed to match these overhangs and replace the part of the YB_TATA minimal promoter that was removed when linearizing the backbone. The ligation reactions comprise 2 μL T4 ligase buffer, 1 μL T4 Ligase (400 U/μL; NEB), 1 μL backbone (typically around 50 ng), 3 μL of each insert (diluted to 20 nM), and water to a total volume of 20 μL. The specific binding sites used for each natural TF are as follows: cJun (reporter 1, AP-1 binding site TGAGTCA, SEQ ID NO: 10; reporter 2, cJun dimer binding site TTACCTCA, SEQ ID NO: 11), BATF (BATF/IRF binding site GAAATGAGTCA, SEQ ID NO: 12), Tbet (palindromic Tbet binding site AATTTCACACCTAGGTGTGAAATT, SEQ ID NO: 13), NFAT (NFAT binding site ACGCCTTCTGTATGAAACAGTTTTTCCTCC, SEQ ID NO: 14).

Cloning transposon vectors and landing pad integration vectors: Both transposon vectors (for genomic integration of receptors and sometimes reporters) and landing pad integration vectors (for genomic integration of reporters) were assembled through a BbsI-mediated Golden Gate reaction based on the previously published mMoClo system (Duportet, X. et al. (2014) Nucleic Acids Res 42, 13440-13451). Each 20 μL reaction comprised 2 μL 10×T4 ligase buffer, 2 μL 10×BSA (1 mg/mL stock), 0.8 μL BbsI-HF (NEB), 0.8 μL T4 DNA Ligase (400 U/μL stock), 20 fmol integration vector backbone, and 40 fmol of each transcription unit and linker plasmid to be inserted. The backbone for piggyBac transposon vectors was pHIE426, for sleeping beauty transposon vectors was pHIE430, and for landing pad integration vectors was pPD1178. Transposon vector assemblies typically included transcriptional units encoding receptors, constitutive fluorescent markers, antibiotic selection markers, and sometimes a fluorescent reporter regulated by a synthetic TF-responsive promoter. Landing pad integration vector assemblies typically included transcriptional unit(s) encoding fluorescent reporters regulated by synthetic TF-responsive promoters, constitutive fluorescent markers, and antibiotic selection markers. The Golden Gate reaction was incubated at 37° C. for 15 min, then subjected to 55 iterations of thermocycling (37° C. for 5 min, 16° C. for 3 min, repeat), followed by 37° C. for 15 min, 50° C. for 5 min, 80° C. for 10 min to terminate the reactions; then the mixture was cooled to room temperature and placed on ice prior to immediate transformation into NEB Stable E. coli. Because all three backbones used here contain the crt operon, colonies that were transformed with unmodified backbone appeared red after 24 h of growth and were discarded.

Plasmid preparation: In most cases, TOP10 or NEB Stable E. coli were grown overnight in 100 mL of LB with the appropriate selective antibiotic. The following morning, cells were pelleted at 3000×g for 10 min and then resuspended in 4 mL of a solution of 25 mM Tris pH 8.0, 10 mM EDTA, and 15% sucrose. Cells were lysed for 15 min by addition of 8 mL of a solution of 0.2 M NaOH and 1% SDS, followed by neutralization with 5 mL of 3 M sodium acetate (pH 5.2). Precipitate was pelleted by centrifugation at 9000×g for 20 min. Supernatant was decanted and treated with 3 μL of RNAse A (Thermo Fisher) for 1 h at 37° C. 5 mL of phenol chloroform was added, and the solution was mixed and then centrifuged at 7500×g for 20 min. The aqueous layer was removed and subjected to another round of phenol chloroform extraction with 7 mL of phenol chloroform. The aqueous layer was then subjected to an isopropanol precipitation (41% final volume isopropanol, 10 min at room temperature, 9000×g for 20 min), and the pellet was briefly dried and resuspended in 420 μL of water. The DNA mixture was incubated on ice for at least 12 h in a solution of 6.5% PEG 20,000 and 0.4 M NaCl (1 mL final volume). DNA was precipitated with centrifugation at maximum speed for 20 min. The pellet was washed once with ethanol, dried for several h at 37° C., and resuspended for several h in TE buffer (10 mM Tris, 1 mM EDTA, pH 8.0). DNA purity and concentration were confirmed using a Nanodrop 2000 (Thermo Fisher). In some cases, TOP10 or NEB Stable E. coli were grown overnight in 50-100 mL of LB with the appropriate selective antibiotic and DNA was prepped using a ZymoPURE II Plasmid Midiprep Kit (Zymo #D4201) by following the manufacturer's instructions. In a given experiment, all receptor variants being compared were prepped using the same method. For some transposon vectors and reporter landing pad integration vectors, 10 mL of NEB Stable E. coli were grown overnight in LB with the appropriate selective antibiotic and DNA was prepped using a ZymoPURE Plasmid Miniprep Kit (Zymo #D4210) by following the manufacturer's instructions.

Cell culture: The HEK293FT cell line was purchased from Thermo Fisher/Life Technologies (RRID: CVCL_6911). The HEK293FT-LP was authenticated by flow cytometric analysis of EYFP expression, which was shown to be homogenous and stable over time-a pattern which is consistent with the original description of this cell line. HEK293FT and HEK293FT-LP cells were cultured in DMEM (Gibco 31600-091) with 10% FBS (Gibco 16140-071), 6 mM L-glutamine (2 mM from Gibco 31600-091 and 4 mM from additional Gibco 25030-081), penicillin (100 U/μL), and streptomycin (100 μg/mL) (Gibco 15140122), in a 37° C. incubator with 5% CO₂. HEK293FT and HEK293FT-LP cells were subcultured at a 1:5 to 1:10 ratio every 2-3 d using Trypsin-EDTA (Gibco 25300-054). All HEK293FT cell lines generated by engineering either the HEK293FT or HEK293FT-LP parent cell lines were cultured in the same way. The Jurkat T cell line (ATCC TIB-152) was purchased from ATCC. Jurkat T cells were cultured in Rosewell Park Memorial Institute Medium (RPMI 1640, Gibco 31800-105) supplemented with 10% FBS (Gibco 16140-071), 4 mM L-glutamine (Gibco 25030-081), penicillin (100 U/μL), and streptomycin (100 μg/mL) (Gibco 15140122). Jurkat T cells were subcultured at a 1:5 or 1:10 ratio every 2-3 d. Cells were maintained at 37° C. with 5% CO₂. All Jurkat cell lines generated by engineering the parent cell line were cultured in the same way. The HEK293FT, HEK293FT-LP, and Jurkat cell lines tested negative for mycoplasma with the MycoAlert Mycoplasma Detection Kit (Lonza Cat #LT07-318).

Transient co-transfection of HEK293FTs: Transient transfection of HEK293FT cells was conducted using the calcium phosphate method. Cells were plated at a minimum density of 1.0×10⁵ cells per well in a 24-well plate in 0.5 ml DMEM, supplemented as described above. For western blot and surface staining experiments, cells were plated at a minimum density of 2.0×10⁵ cells per well in a 12-well plate in 1 ml DMEM. After about 24 h, by which time the cells had adhered to the plate and grown to about 50% confluency in the well, the cells were transfected. Plasmids (up to 500 ng DNA for 24-well plates and up to 1,000 ng DNA for 12-well plates) were mixed in H2O, and 2 M CaCl2 was added to a final concentration of 0.3 M CaCl2. This mixture was added dropwise to an equal-volume solution of 2×HEPES-buffered saline (280 mM NaCl, 0.05 M HEPES, 1.5 mM Na2HPO4) and gently pipetted up and down four times. After 4 min, the solution was mixed vigorously by pipetting ten times. Next, 100 μl of this mixture was added dropwise to the plated cells in 24-well plates (or 200 μl to 12-well plates), and the plates were gently swirled. All receptor plasmid masses were calculated by normalizing to a copy number of 4.22×10⁹ for 24-well plates and 8.44×10⁹ for 12-well plates, approximately 35-40 ng per 24-well and 70-80 ng per 12-well. These numbers were determined empirically from prior experiments involving rapamycin-sensing receptors and the dose scales with well size. For conditions that received co-expressed ligands, 20 ng of ligand-expressing plasmid was included per 24-well. The total mass of all samples in an experiment was held constant by supplementing with empty vector filler DNA (pHIE298). The next morning, the medium was aspirated and replaced with fresh medium.

Signaling assays with co-expressed ligand: Cells were transfected as described above. The morning after transfection, medium was aspirated and replaced with fresh medium. Typically, at 36-48 h post-transfection and at least 24 h post-media change, cells were harvested. Cells were harvested for flow cytometry by washing with PBS pH 7.4 and using 0.05% Trypsin-EDTA (Thermo Fisher Scientific #25300120) for 5 min followed by quenching with medium. Cell suspensions were pipetted and added to 1 mL of FACS buffer (PBS pH 7.4, 2-5 mM EDTA, 0.1% BSA). Cells were spun at 150×g for 5 min, supernatant was decanted, and fresh FACS buffer was added.

Signaling assays with external, recombinant ligand: For experiments with transfected receptors, cells were transfected using lipofectamine LTX based on the manufacturer's recommendations and no co-expressed ligand was included. Briefly, cells were plated in 12-well plates at a density of 2.0×10⁵ cells per well in complete DMEM the left to adhere. After 24 h, cells were transfected with 100 μl of mix containing 8.44×10⁹ receptor plasmid copies and supplemented with filler DNA (pHIE298) to reach a total mass of 1,000 ng. The morning after transfection, medium was aspirated and replaced with fresh medium with or without recombinant ligand, typically 100 ng/mL VEGF (Biolegend #583706) or 250 ng/ml IL-10 (Biolegend #573206). The next day, cells were passaged at a 1:2 subculture ratio and treated again with or without ligand. The next day, cells were harvested for flow cytometry by washing with PBS pH 7.4 and using 0.05% Trypsin-EDTA (Thermo Fisher Scientific #25300120) for 5 min followed by quenching with medium. Cell suspensions were pipetted and added to 1 mL of FACS buffer (PBS pH 7.4, 2-5 mM EDTA, 0.1% BSA). Cells were spun at 150×g for 5 min, supernatant was decanted, and fresh FACS buffer was added.

For experiments with stably expressed receptors, cells were plated in 24-well plates at a density of 0.75×10⁵ cells per well. The next morning, medium was aspirated and replaced with fresh medium with or without recombinant ligand, typically 100 ng/mL VEGF (Biolegend #583706) or 250 ng/mL IL-10 (Biolegend #573206). Cells were left alone for 48 h before harvesting for flow cytometery. Cells were harvested for flow cytometry by washing with PBS pH 7.4 and using 0.05% Trypsin-EDTA (Thermo Fisher Scientific #25300120) for 5 min followed by quenching with medium. Cell suspensions were pipetted and added to 1 mL of FACS buffer (PBS pH 7.4, 2-5 mM EDTA, 0.1% BSA). Cells were spun at 150×g for 5 min, supernatant was decanted, and fresh FACS buffer was added.

Time course microscopy signaling assays with external ligand: For time course microscopy experiments, cells were plated at a density of 0.25×10⁵ cells per well in 48-well plates. The next morning, IL-10 was diluted in serum free (incomplete) DMEM to a concentration of 25 ng/μL and 5 μL of this mix was added to the side of each well to make the final concentration 250 ng/μL. For untreated wells, 5 μL of serum free DMEM was added. Plates were immediately placed in the microscope (Keyence BZ-X800E), well positions were chosen using the brightfield channel, and the time course was started. Plates were held in a stage top incubator (TOKAI HIT, INU-KIW-F1) to maintain humidity, a temperature of 37° C., and CO₂ concentration of 5%. Images were taken every 2 hrs. for 48 hrs. A BZ-X GFP filter (Ex 470/40 nm, Em 525/50 nm, dichroic 495 nm) was used to measure mNeonGreen fluorescence and a BZ-X Texas Red filter (Ex 560/40 nm, Em 630/75 nm, dichroic 585 nm) was used to measure DsRedExpress2 reporter fluorescence. The following exposure times were used: ⅕ sec for mNeonGreen, 1/40 sec for DsRedExpress2, 1/7500 sec for brightfield using 25% transmitted light with oblique illumination. Images were acquired using BZ Series Application software v01.01.00.17 and PlanApo 4×objective with a numerical aperture of 0.2. Images were not modified or corrected in any way. Images were processed using custom software in MATLAB to extract the fluorescence in each channel per pixel, normalize DsRedExpress2 to mNeonGreen fluorescence on a pixel-by-pixel basis, and calculate the average normalized fluorescence across the entire image for each field of view at each timepoint.

Signaling assays with external, surface bound ligand: A Nunc Immobilizer Streptavidin 96-well plate (Thermo Scientific #436014) was pre-washed with 300 μL/well BSA buffer (1 mg/mL BSA in PBS) 3×5 min at 37° C. Next, 100 μL per well of biotinylated cytokine solution was added at the specified concentrations (0, 1, 5, and 10 μg/mL biotinylated VEGFA-165 in BSA buffer). The plate was incubated with gentle agitation for 2 h at room temperature (22° C.). The wells were then aspirated and washed with 300 μL of BSA buffer per well for 5 min at 37° C. Wells were next washed with complete DMEM 5×5 min at 37° C. The final wash volume was aspirated, and cells were plated at a density of 1.6×10+ cells/well in complete DMEM, with a total volume of 200 μL per well. The cells were incubated at 37° C. for approximately 48 h before harvesting for flow. Cells that stably express VEGFR NatE MESA receptors and the reporter were harvested for flow cytometry by washing with PBS pH 7.4 and using 0.05% Trypsin-EDTA (Thermo Fisher Scientific #25300120) for 5 min followed by quenching with medium. Cell suspensions were pipetted and added to 1 mL of FACS buffer (PBS pH 7.4, 2-5 mM EDTA, 0.1% BSA). Cells were spun at 150×g for 5 min, supernatant was decanted, and fresh FACS buffer was added.

Immunohistochemistry (surface staining): For surface staining to quantify MESA receptor surface expression, HEK293FT cells were plated at 2×10⁵ cells per well in 1 mL DMEM in 12-well plates 24 h before transfection and transfected as described above, using 200 μL transfection reagent per well. The receptor plasmid copy number for individual receptors matches the copy number used in functional assays, scaled up to 12-well format (8.44×10⁹ receptor plasmid copies). At 36-48 h after transfection, cells were harvested with 500 μl FACS buffer and spun at 150×g at 4° C. for 5 min. Supernatant was decanted, and 50 μL fresh FACS buffer and 10 μL human IgG (Human IgG Isotype Control, ThermoFisher Scientific #02-7102, RRID: AB_2532958, stock concentration 1 mg/mL) was added. Cells were incubated in this mixture at 4° C. for 5 min. Next, 5 μL FLAG tag antibody (Anti-DDDDK-APC, Abcam ab72569, RRID: AB_1310127) was added at a concentration of 0.5 ug per sample and cells incubated at 4° C. for 30 min. Following incubation, 1 mL of FACS buffer was added, cells were spun at 150×g at 4° C. for 5 min, and supernatant was decanted. This wash step was repeated two more times to total three washes. After decanting supernatant in the final wash, 1-3 drops of FACS buffer were added.

For surface staining to quantify CAR expression, HEK293FTs that were genomically engineered to contain the circuit components for CAR expression were plated at 2× 10⁵ cells per well in 1 mL DMEM and treated with or without 250 ng/ml recombinant IL-10 for 48 h before harvesting. Cells were harvested as described for MESA receptor surface staining. For HEK293FTs transfected with circuit components for CAR expression, cell plating and harvest is identical to that described for MESA receptor surface staining. For Jurkats that were genomically engineered to contain the circuit components for CAR expression, cells were plated at 2× 10⁵ cells/mL in 1 mL in 12-well plates with or without 250 ng/ml recombinant IL-10 and incubated for 48 h. Jurkats were harvested by collecting the full volume remaining in each well, diluting in 1 mL FACS buffer, and spinning down at 150×g at 4° C. for 5 min. Supernatant was decanted, and 50 μL fresh FACS buffer and 10 μL human IgG (Human IgG Isotype Control, ThermoFisher Scientific #02-7102, RRID: AB_2532958, stock concentration 1 mg/mL) was added. Cells were incubated in this mixture at 4° C. for 5 min. Next, 2.5 μL anti-hEGF tag antibody (Anti-hEGF-biotin, R&D Systems BAF236) was added at a concentration of 0.5 ug per sample and cells incubated at 4° C. for 20 min. Following incubation, 1 mL of FACS buffer was added, cells were spun at 150×g at 4° C. for 5 min, and supernatant was decanted. Next, 2.5 μL Strep-APC (Abcam ab 134362) was added at a concentration of 0.5 ug per sample and cells incubated at 4° C. for 20 min. Following incubation, 1 mL of FACS buffer was added, cells were spun at 150×g at 4° C. for 5 min, and supernatant was decanted. This wash step was repeated two more times to total three washes. After decanting supernatant in the final wash, 1-3 drops of FACS buffer were added. For Jurkats, FACS buffer contained 3 μM DAPI for viability staining.

Analytical flow cytometry: Flow cytometry was run on a BD LSR Fortessa Special Order Research Product. Lasers and filter sets used for data acquisition are listed in Table 2 below. Approximately 3,000-10,000 single, transfected cells were analyzed per sample in transfection experiments. Transfected cells were identified using a separate, single transfection control fluorescent protein or multiple fluorescent proteins encoded on receptor plasmids (e.g., mNeonGreen and mTagBFP2). In cases where the transfected cells are landing pad engineered cells, a constitutive miRFP720 gene is included in landing pad cargo so cells are first identified by miRFP720 expression before setting transfection gate(s). In cases where the cells were engineered with transposons, a constitutive mNeonGreen gene is included in the transposon so engineered cells were identified by mNeonGreen fluorescence.

TABLE 2
Instrument specifications for analytical flow cytometry.

| | Fluorescent | Parameter/Channel | Excitation | Filter |
|---|---|---|---|---|
| Instrument | protein | name | laser | set |
| BD LSR | mTagBFP2 | Pacific Blue | Violet, | 450/50 |
| Fortessa | | | 405 nm | |
| | mNeonGreen | FITC | Blue, | 505LP, |
| | | | 488 nm | 530/30 |
| | DsRedExpress2 | PE-Texas Red | Yellow | 600LP, |
| | | | Green, | 610/20 |
| | | | 550 nm | |
| | miRFP720 | Alexa Fluor 750 | Far Red, | 690LP, |
| | | | 685 nm | 730/45 |

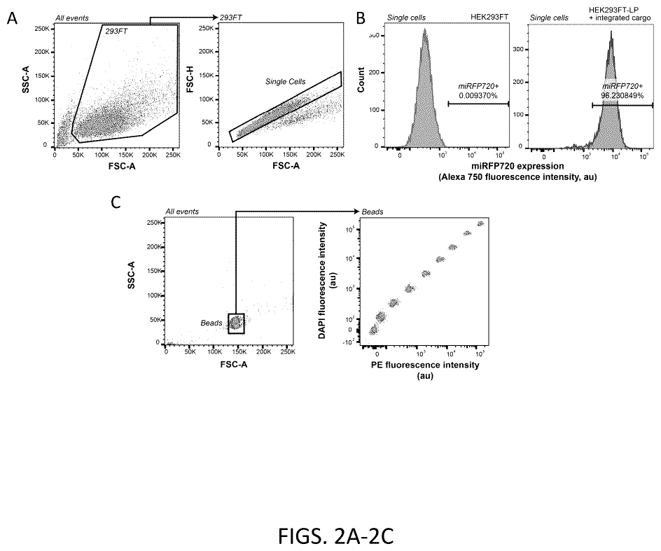

Samples were analyzed using FlowJo v10 software (FlowJo, LLC). Fluorescence data were compensated for spectral bleed-through. As shown in FIG. 2, the HEK293FT cell population was identified by SSC-A versus FSC-A gating, and singlets were identified by FSC-A versus FSC-H gating (FIG. 2A). To distinguish transfected from non-transfected cells, a control sample of cells was generated by transfecting cells with a mass of pcDNA (empty vector) equivalent to the mass of DNA used in other samples in the experiment. For the single-cell subpopulation of the pcDNA-only sample, a gate was made to identify cells that were positive for the constitutive fluorescent protein(s) used as a transfection control in other samples, such that the gate included no more than 1% of the non-fluorescent cells (FIG. 2B). A similar approach was used to identify genomically modified miRFP720 or mNeonGreen expressing HEK293FTs. The Jurkat cell population was identified by SSC-A versus FSC-A gating, and singlets were identified by FSC-A versus FSC-H gating. Live cells were identified by inclusion of a DAPI viability stain such that DAPI+ (dead) cells were excluded from analysis. Genomically engineered Jurkats were identified by mNeonGreen expression by drawing a gate on un-modified Jurkats such that the gate included no more than 1% of the non-fluorescent cells.

Generation of stable receptor cell lines: To generate HEK293FT cell lines, from exponentially growing cells, 0.5×10⁵ cells were plated per well (0.5 mL medium) in 24-well format, and cells were cultured for 24 h to allow cells to attach and spread. HyPBase (hyperactive PiggyBac transposase, pHIE425) or SB100X (hyperactive Sleeping Beauty transposase, pHIE429) encoded on pcDNA-based plasmids were co-transfected with their respective transposon vector by lipofection with Lipofectamine LTX with PLUS Reagent (ThermoFisher 15338100). 100 ng of transposase expression vector was mixed with 500 ng of reporter-containing integration vector, 0.6 μL of PLUS reagent, and enough OptiMEM (ThermoFisher/Gibco 31985062) to bring the mix volume up to 25 μL. In a separate tube, 2 μL of LTX reagent was mixed with 23 μL of OptiMEM. The DNA/PLUS reagent mix was added to the LTX mix, pipetted up and down four times, and then incubated at room temperature for 5 min. 50 μL of this transfection mix was added dropwise to each well of cells and mixed by gentle swirling. Cells were cultured until the well was ready to split (typically 3 d), without any media changes.

To begin selection of HEK293FT cells that successfully integrated the transposon vector, cells were harvested from the 24-well plate when confluent by trypsinizing and transferring to a single well of a 12-well plate in 1 mL of medium supplemented with 1 μg/mL puromycin (Invivogen ant-pr) or 200 μg/mL hygromycin (Millipore, #400053) depending on the vector. Cells were trypsinized daily (typically 3 d) until cell death was no longer evident. Cells were cultured in medium supplemented with puromycin through exponential expansion until reaching a confluent 10 cm dish, upon which cells were frozen. Selective pressure was maintained when culturing these cells but not included during experiments.

For sorting experiments, HEK293FT cells were harvested by trypsinizing, resuspended at approximately 10⁷ cells per mL in pre-sort medium (DMEM with 10% FBS, 25 mM HEPES (Sigma H3375), and 100 μg/mL gentamycin (Amresco 0304)), and held on ice until sorting was performed. Cells were sorted using a BD FACS Aria 4-laser Special Order Research Product (Robert H. Lurie Cancer Center Flow Cytometry Core). Details on laser and channel configurations can be found in Table 3, below. The first sorting strategy was as follows: single cells were first gated to exclude all mNeonGreen negative cells (as mNeonGreen is a constitutive marker in the transposon and successfully engineered, non-silenced cells should express this protein). Then, the mNeonGreen positive population was broken into octiles that each contained 12.5% of the mNeonGreen positive population and the top (brightest mNeonGreen) four octiles were sorted. The second sorting strategy required an additional gate for exclusion of reporter (DsRedExpress2) positive cells to isolate low background cells and the same octile sorting strategy was applied to this mNeonGreen+/DsRedExpress2-population. The top four mNeonGreen octiles were sorted. 50,000 cells were collected in post-sort medium (DMEM with 20% FBS, 25 mM HEPES, and 100 μg/mL gentamycin), and cells were held on ice until they could be centrifuged at 150×g for 5 min, resuspended in 0.5 mL complete medium supplemented with 100 μg/mL gentamycin, and plated in one well of a 24-well plate. Cells were maintained in gentamycin for 7 d after sorting during expansion before banking. Cells were thawed for use in experiments in this study.

TABLE 3
Instrument specifications for fluorescence-activated cell sorting.

| | Fluorescent | Parameter/Channel | Excitation | Filter |
|---|---|---|---|---|
| Instrument | protein | name | laser | set |
| BD FacsAria | mNeonGreen | FITC | Blue, | 505LP, |
| IIu | | | 488 nm | 530/30 |
| | DsRedExpress2 | PE-Texas Red | Yellow | 600LP, |
| | | | Green, | 610/20 |
| | | | 561 nm | |
| | miRFP720 | APC-Cy7 | Red, | 690LP, |
| | | | 633 nm | 730/45 |

To generate Jurkat cell lines, from exponentially growing cells, 0.5×10⁵ cells were plated per well (0.5 mL medium) in 24-well format at the time of transfection. HyPBase (hyperactive PiggyBac transposase, pHIE425) was co-transfected with a transposon vector by lipofection with Lipofectamine LTX with PLUS Reagent (ThermoFisher 15338100). 100 ng of transposase expression vector was mixed with 500 ng of reporter-containing integration vector, 0.6 μL of PLUS reagent, and enough OptiMEM (ThermoFisher/Gibco 31985062) to bring the mix volume up to 25 μL. In a separate tube, 2 μL of LTX reagent was mixed with 23 μL of OptiMEM. The DNA/PLUS reagent mix was added to the LTX mix, pipetted up and down four times, and then incubated at room temperature for 30-60 min. 50 μL of this transfection mix was added dropwise to each well of cells and mixed by gentle swirling. Cells were cultured until the well was ready to split (typically 3 d), without any media changes.

To begin selection of Jurkat cells that successfully integrated the transposon vector, cells were harvested from the 24-well plate and transferred to a single well of a 12-well plate in 1 mL of fresh medium. After 24 h, the entire contents of the 12-well was transferred to a 6-well and supplemented with 0.2 ug/mL puromycin (Invivogen ant-pr). Cells were analyzed daily and spun at 125×g for 5 min at 4° C. approximately once per week to fully refresh media. Every 3 days, 1 mL puro-containing medium was added. Once cells were exponentially growing, they were transferred up to 10 cm dishes in medium supplemented with puromycin, passaged once without puromycin, and frozen. Selective pressure was maintained when culturing these cells but not included during experiments.

To sort, Jurkat cells were harvested, spun down at 125×g for 5 min at 4° C. before resuspending at approximately 10⁷ cells per mL in pre-sort medium (RPMI with 10% FBS, 25 mM HEPES (Sigma H3375), and 100 μg/mL gentamycin (Amresco 0304)), and held on ice until sorting was performed. Cells were sorted using a BD FACS Aria 4-laser Special Order Research Product (Robert H. Lurie Cancer Center Flow Cytometry Core). Details on laser and channel configurations can be found in Table 3, above. The sorting strategy was as follows: single cells were first gated to exclude all mNeonGreen negative cells (as mNeonGreen is a constitutive marker in the transposon and successfully engineered, non-silenced cells should express this protein). Then, the mNeonGreen positive population was broken into octiles that each contained 12.5% of the mNeonGreen positive population and the top (brightest mNeonGreen) octile was sorted. 50,000 cells were collected in post-sort medium (DMEM with 20% FBS, 25 mM HEPES, and 100 μg/mL gentamycin), and cells were held on ice until they could be centrifuged at 125×g for 5 min at 4° C., resuspended in 0.5 mL complete medium supplemented with 100 μg/mL gentamycin, and plated in one well of a 24-well plate. Cells were maintained in gentamycin for 7 d after sorting during expansion before banking. Cells were thawed for use in experiments in this study.

Generation of stable reporter cell lines: From exponentially growing HEK293LP cells, 0.5×10⁵ cells were plated per well (0.5 mL medium) in 24-well format, and cells were cultured for 24 h to allow cells to attach and spread. Bxb1 recombinase was co-transfected with the integration vector by lipofection with Lipofectamine LTX with PLUS Reagent (ThermoFisher 15338100). 300 ng of BxB1 expression vector was mixed with 300 ng of reporter-containing integration vector, 0.5 μL of PLUS reagent, and enough OptiMEM (ThermoFisher/Gibco 31985062) to bring the mix volume up to 25 μL. In a separate tube, 1.9 μL of LTX reagent was mixed with 23.1 μL of OptiMEM. The DNA/PLUS reagent mix was added to the LTX mix, pipetted up and down four times, and then incubated at room temperature for 5 min. 50 μL of this transfection mix was added dropwise to each well of cells and mixed by gentle swirling. Cells were cultured until the well was ready to split (typically 3 d), without any media changes.

To begin selection of cells that successfully integrated the reporter integration vector, cells were harvested from the 24-well plate when confluent by trypsinizing and transferring to a single well of a 6-well plate in 2 mL of medium supplemented with 1 μg/mL puromycin (Invivogen ant-pr). Cells were trypsinized daily (typically 3 d) until cell death was no longer evident. Cells were cultured in medium supplemented with puromycin until the 6-well was confluent and cells were exponentially growing. Cells were then selected with 6 μg/ml blasticidin (Alfa Aesar/ThermoFisher J61883) for 7 d. Cells were cultured in both puromycin and blasticidin to maintain selective pressure until flow sorting.

To sort, cells were harvested by trypsinizing, resuspended at approximately 10⁷ cells per mL in pre-sort medium (DMEM with 10% FBS, 25 mM HEPES (Sigma H3375), and 100 μg/mL gentamycin (Amresco 0304)), and held on ice until sorting was performed. Cells were sorted using a BD FACS Aria 4-laser Special Order Research Product (Robert H. Lurie Cancer Center Flow Cytometry Core). The sorting strategy was as follows: single cells were first gated to exclude all EYFP positive cells (as EYFP positive cells still have an intact landing pad locus, suggesting a mis-integration event occurred) and to include only miRFP720+ cells. EYFP expression was measured using the FITC channel and miRFP720 expression was measured using a modified APC-Cy7 channel. Then a gate was drawn on miRFP720 expression to capture the 88th to 98th percentile of miRFP720-expressing cells (the top 2% were excluded to exclude cells suspected to possess two or more integrated copies of the cargo vector). 50,000 cells were collected in post-sort medium (DMEM with 20% FBS, 25 mM HEPES, and 100 μg/mL gentamycin), and cells were held on ice until they could be centrifuged at 150×g for 5 min, resuspended in 0.5 mL complete medium supplemented with 100 μg/mL gentamycin, and plated in one well of a 24-well plate. Cells were maintained in gentamycin for 7 d after sorting during expansion before banking. Cells were thawed for use in experiments in this study.

Quantification of reporter output: MESA signaling was quantified by measuring the expression of a downstream fluorescent reporter protein, DsRedExpress2, regulated by a synTF-inducible promoter. The mean fluorescence intensity (MFI) for each relevant channel (as defined in Table 2, above) of the single cell transfected population (transfection control marker+) or the single cell transposon modified population (mNeonGreen+) was calculated and exported for further analysis. To calculate reporter expression, MFI in the PE-Texas Red channel was averaged across three biological replicates. The MFI was converted to Molecules of Equivalent PE-Texas Red (MEPTRs); as shown in FIG. 2C, to determine conversion factors for MFI to MEPTRs, UltraRainbow Calibration Particles (Spherotech #URCP-100-2H) were run with each flow cytometry experiment. These reagents contain nine subpopulations of beads, each with a known number of various fluorophores. The total bead population was identified by FSC-A vs. SSC-A gating, and bead subpopulations were identified through two fluorescent channels. MEPTR values corresponding to each subpopulation were supplied by the manufacturer. A calibration curve was generated for the experimentally determined MFI vs. the manufacturer supplied MEPTRs, and a linear regression was performed with the constraint that 0 MFI equals 0 MEPTRs. The slope from the regression was used as the conversion factor, and error was propagated. Fold differences were calculated by dividing reporter expression with ligand treatment by the reporter expression without ligand treatment. Standard error was propagated through all calculations.

Western blotting: For western blotting to detect MESA receptor expression, HEK293FT cells were plated at 2×10⁵ cells per well in 1 mL DMEM in 12-well plates 24 h before transfection and transfected as above, using 200 μL transfection reagent per well (the reaction scales with the volume of medium). At 36-48 h after transfection, cells were lysed with 250 μL RIPA (150 mM NaCl, 50 mM Tris-HCl pH 8.0, 1% Triton X-100, 0.5% sodium deoxycholate and 0.1% sodium dodecyl sulfate) with protease inhibitor cocktail (Pierce/Thermo Fisher #A32953) and incubated on ice for 30 min. Lysate was cleared by centrifugation at 14 000×g for 20 min at 4° C., and supernatant was harvested. A BCA assay was performed to determine protein concentration, and after a 10 min incubation in Laemmli buffer (final concentration 60 mM Tris-HCl pH 6.8, 10% glycerol, 2% sodium dodecyl sulfate, 100 mM dithiothreitol and 0.01% bromophenol blue) at 70° C., protein (10-25 ug, the maximum amount of protein that could be loaded to keep protein mass constant across all samples on a gel) was loaded onto a 4-15% Mini-PROTEAN TGX Precast Protein Gel (Bio-Rad) and run at 50 V for 10 min followed by 100 V for at least 1 h. Wet transfer was performed onto an Immuno-Blot PVDF membrane (Bio-Rad) for 45 min at 100 V. Ponceau-S staining was used to confirm protein transfer.

For detection of MESA receptors using the N-terminal 3×FLAG epitope tag, membranes were blocked for 30 min with 3% milk in Tris-buffered saline pH 8.0 (TBS pH 8.0:50 mM Tris, 138 mM NaCl, 2.7 mM KCl, HCl to pH 8.0), washed once with TBS pH 8.0 for 5 min and incubated for overnight at 4° C. in primary antibody (Mouse-anti-FLAG M2; Sigma #F1804, RRID: AB_262044), diluted 1:1000 in 3% milk in TBS pH 8.0. Primary antibody solution was decanted, and the membrane was washed once with TBS pH 8.0 and twice with TBS pH 8.0 with 0.05% Tween, for 5 min each. Secondary antibody (HRP-anti-Mouse; CST 7076, RRID: AB_330924), diluted 1:3000 in 5% milk in TBST pH 7.6 (TBST pH 7.6:50 mM Tris, 150 mM NaCl, HCl to pH 7.6, 0.1% Tween), was applied for 1 h at room temperature, and the membrane was washed three times for 5 min each time with TBST pH 7.6. After primary and secondary staining and washing, the membrane was incubated with Clarity Western ECL Substrate (Bio-Rad) for 5 min, and then imaged using an Azure c280 (Azure Biosystems). Images were cropped with Illustrator CC (Adobe). No other image processing was employed.

For detection of membrane-bound IL-15 (mbIL-15) using the C-terminal myc epitope tag, membranes were blocked for 30 min with 5% milk in Tris-buffered saline pH 7.6 with 0.1% Tween (TBST pH 7.6:50 mM Tris, 150 mM NaCl, HCl to pH 8.0, 0.1% Tween-20), washed once with TBST pH 7.6 for 5 min and incubated overnight at 4° C. in primary antibody (Mouse-anti-myc; Abcam #ab32, RRID: AB_303599) diluted 1:1000 in 5% milk in TBST pH 7.6. Primary antibody solution was decanted, and the membrane was washed three times with TBST pH 7.6 for 5 min each. Secondary antibody (HRP-anti-Mouse; CST 7076, RRID: AB_330924), diluted 1:3000 in 5% milk in TBST pH 7.6, was applied for 1 h at room temperature, and the membrane was washed three times for 5 min each time with TBST pH 7.6. After primary and secondary staining and washing, the membrane was incubated with Clarity Western ECL Substrate (Bio-Rad) for 5 min, and then imaged using an Azure c280 (Azure Biosystems).

Statistical analyses: ANOVA tests and Tukey's HSD tests were performed using RStudio. Tukey's HSD tests were performed with α=0.05. Pairwise comparisons were made using a two-tailed Welch's t-test, which is a version of Student's t-test in which the variance between samples is treated as not necessarily equal. Two-tailed Welch's 1-tests were performed in GraphPad and Excel. To decrease the false discovery rate, the Benjamini-Hochberg procedure was applied to each set of tests per figure panel; in all tests, after the Benjamini-Hochberg procedure, the null hypothesis was rejected for p-values <0.05. The outcomes for each statistical test are provided in the figure captions.

Development of synthetic biosensors using natural human receptors: To develop strategies for engineering natural ectodomain (NatE) MESA receptors, a panel of native human receptors that represent a variety of receptor classes and signaling mechanisms and that could enable useful biosensing capabilities for soluble disease markers were selected. These receptors included human VEGFR1 and VEGFR2 (for sensing vascular endothelial growth factor, VEGF), IL-10Rα and IL-10Rβ (for sensing interleukin 10, IL-10), TNFR1 and TNFR2 (for sensing tumor necrosis factor, TNF), and TGF-βR1 and TGF-βR2 (for sensing transforming growth factor β, TGF-β). This panel was selected because VEGF, IL-10, and TGF-β are upregulated soluble cues in the tumor microenvironment and TNF and IL-10 are important immunoregulatory molecules that play roles in inflammatory and immunosuppressive states respectively. Additional rationale for each of these systems is provided in subsequent sections. The receptor systems chosen include hetero-associative (different receptor chains associate) and homo-associative (like receptor chains associate) ligand-dependent signaling mechanisms between two or more receptor chains, making them promising candidates for use in a ligand-mediated split protease reconstitution and trans-cleavage (MESA-like) signaling mechanism. A previously validated set of MESA intracellular signaling domains was chosen, including the split tobacco etch virus protease (TEVp) mutant 75S/190K and a synthetic zinc finger-based transcription factor and accompanying linker. Designs that retain the native transmembrane (TMD) and juxtamembrane (JMD) domains for each selected human receptor were chosen because those domains are often highly involved in surface expression, ligand-independent receptor chain association, and ligand-induced signaling events. Designs that replace the native TMD and JMD with the domains that were used in prior work with MESA receptors to validate ligand-induced reconstitution of split TEVp components at the membrane were chosen, including a truncated form of the murine cluster of differentiation 28 (CD28) TMD and a glycine-serine-rich intracellular linker. Finally, variants with either the native signal sequence for each selected human receptor or alternative human signal sequences were chosen, which were hypothesized to improve trafficking to the cell surface if necessary. The two alternative signal sequences are widely used on chimeric antigen receptors (CARs) and synNotch receptors, and they are derived from the human cluster of differentiation 8a (CD8a) T cell receptor and from the human immunoglobulin variable heavy chain (IgG VH). The exploration of design space defined here provides a starting point for evaluating the general prospects of natural receptor conversion.

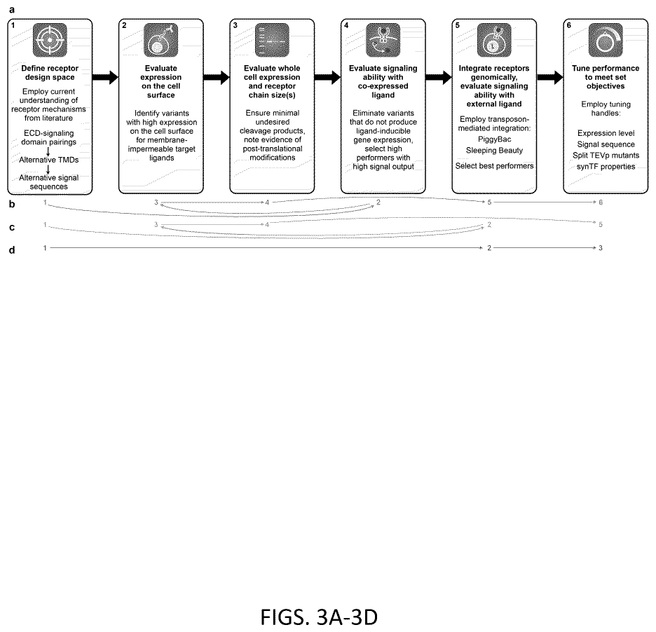

The workflow for converting natural human receptors into synthetic biosensors follows the same general steps for each of VEGFR, IL-10R, TNFR, and TGF-βR as described below. A general schematic of the workflow is shown in FIGS. 3A-3D.

### Example 1—Conversion of VEGFR

VEGF NatE MESA receptors comprise human VEGFR1 and human VEGFR2 signal peptides and ectodomains (hVEGFR1 SP (SEQ ID NO: 15) and ECD (SEQ ID NO: 16): aa1-758, hVEGFR2 SP (SEQ ID NO: 19) and ECD (SEQ ID NO: 20): aa1-764). When the native transmembrane domains were used, five amino acids of the intracellular juxtamembrane domain were retained (hVEGFR1 JMD: aa781-785 (SEQ ID NO: 18), hVEGFR2 JMD: aa 786-790 (SEQ ID NO: 22)). In some variants, the mouse CD28 transmembrane domain (SEQ ID NO: 28) was used in place of the hVEGFR1 and hVEGFR2 transmembrane domains (hVEGFR1 TMD: aa759-780 (SEQ ID NO: 17), hVEGFR2 TMD: aa765-785 (SEQ ID NO: 21), mCD28 TMD: aa154-174 (SEQ ID NO: 28)) with a juxtamembrane domain consisting of GGGSGG (SEQ ID NO: 29). When alternative signal peptides were used, the natural hVEGFR1 and hVEGFR2 signal peptides were exchanged for the signal peptides of the human CD8a receptor and the human IgG variable heavy chain (hVEGFR1 SP: aa1-26 (SEQ ID NO: 15), hVEGFR2 SP: aa1-19 (SEQ ID NO: 19), hCD8a SP: aa1-21 (SEQ ID NO: 23), hIgGVH SP: aa1-19 (SEQ ID NO: 24)). Sequences are found in Tables 4 and 5.

TABLE 4
VEGFR Sequences

| | SEQ ID | |
|---|---|---|
| Name | NO | Sequence |
| VEGFR1 SP | 15 | MVSYWDTGVLLCALLSCLLLTGSSSG |
| VEGFR1 ED | 16 | SKLKDPELSLKGTQHIMQAGQTLHLQCRGEAAHKWS |
| | | LPEMVSKESERLSITKSACGRNGKQFCSTLTLNTAQA |
| | | NHTGFYSCKYLAVPTSKKKETESAIYIFISDTGRPFVE |
| | | MYSEIPEIIHMTEGRELVIPCRVTSPNITVTLKKFPLDTL |
| | | IPDGKRIIWDSRKGFIISNATYKEIGLLTCEATVNGHLY |
| | | KTNYLTHRQTNTIIDVQISTPRPVKLLRGHTLVLNCTA |
| | | TTPLNTRVQMTWSYPDEKNKRASVRRRIDQSNSHANI |
| | | FYSVLTIDKMQNKDKGLYTCRVRSGPSFKSVNTSVHI |
| | | YDKAFITVKHRKQQVLETVAGKRSYRLSMKVKAFPS |
| | | PEVVWLKDGLPATEKSARYLTRGYSLIIKDVTEEDAG |
| | | NYTILLSIKQSNVFKNLTATLIVNVKPQIYEKAVSSFPD |
| | | PALYPLGSRQILTCTAYGIPQPTIKWFWHPCNHNHSEA |
| | | RCDFCSNNEESFILDADSNMGNRIESITQRMAIIEGKN |
| | | KMASTLVVADSRISGIYICIASNKVGTVGRNISFYITDV |
| | | PNGFHVNLEKMPTEGEDLKLSCTVNKFLYRDVTWILL |
| | | RTVNNRTMHYSISKQKMAITKEHSITLNLTIMNVSLQ |
| | | DSGTYACRARNVYTGEEILQKKEITIRDQEAPYLLRNL |
| | | SDHTVAISSSTTLDCHANGVPEPQITWFKNNHKIQQEP |
| | | GIILGPGSSTLFIERVTEEDEGVYHCKATNQKGSVESS |
| | | AYLTVQGTSDKSNLE |
| VEGFR1 TMD | 17 | LITLTCTCVAATLFWLLLTLFI |
| VEGFR1 JMD | 18 | RKMKR |
| VEGFR2 SP | 19 | MQSKVLLAVALWLCVETRA |
| VEGFR2 ED | 20 | ASVGLPSVSLDLPRLSIQKDILTIKANTTLQITCRGQRD |
| | | LDWLWPNNQSGSEQRVEVTECSDGLFCKTLTIPKVIG |
| | | NDTGAYKCFYRETDLASVIYVYVQDYRSPFIASVSDQ |
| | | HGVVYITENKNKTVVIPCLGSISNLNVSLCARYPEKRF |
| | | VPDGNRISWDSKKGFTIPSYMISYAGMVFCEAKINDES |
| | | YQSIMYIVVVVGYRIYDVVLSPSHGIELSVGEKLVLNC |
| | | TARTELNVGIDFNWEYPSSKHQHKKLVNRDLKTQSGS |
| | | EMKKFLSTLTIDGVTRSDQGLYTCAASSGLMTKKNST |
| | | FVRVHEKPFVAFGSGMESLVEATVGERVRIPAKYLGY |
| | | PPPEIKWYKNGIPLESNHTIKAGHVLTIMEVSERDTGN |
| | | YTVILTNPISKEKQSHVVSLVVYVPPQIGEKSLISPVDS |
| | | YQYGTTQTLTCTVYAIPPPHHIHWYWQLEEECANEPS |
| | | QAVSVTNPYPCEEWRSVEDFQGGNKIEVNKNQFALIE |
| | | GKNKTVSTLVIQAANVSALYKCEAVNKVGRGERVISF |
| | | HVTRGPEITLQPDMQPTEQESVSLWCTADRSTFENLT |
| | | WYKLGPQPLPIHVGELPTPVCKNLDTLWKLNATMFS |
| | | NSTNDILIMELKNASLQDQGDYVCLAQDRKTKKRHC |
| | | VVRQLTVLERVAPTITGNLENQTTSIGESIEVSCTASGN |
| | | PPPQIMWFKDNETLVEDSGIVLKDGNRNLTIRRVRKE |
| | | DEGLYTCQACSVLGCAKVEAFFIIEGAQEKTNLE |
| VEGFR2 TMD | 21 | IIILVGTAVIAMFFWLLLVII |
| VEGFR2 JMD | 22 | LRTVK |

TABLE 5
Alternative Sequences

| | SEQ | |
|---|---|---|
| | ID | |
| Name | NO | Sequence |
| Human CD8a | 23 | MALPVTALLLPLALLLHAARP |
| derived signal | | |
| peptide | | |
| Human IgG VH | 24 | MEFGLSWLFLVAILKGVQC |
| signal peptide | | |
| Truncated | 25 | LVVVGGVLACYSLLVTVAFII |
| human CD28 | | |
| derived TMD | | |
| Full length | 26 | FWVLVVVGGVLACYSLLVTVAFIIFWV |
| human CD28 | | |
| derived TMD | | |
| Human CD28 | 27 | RSKRSRLLH |
| derived JMD | | |
| Mouse CD28 | 28 | LVVVAGVLFCYGLLVTVALCV |
| derived TMD | | |
| Alternative | 29 | GGGSGG |
| JMD | | |

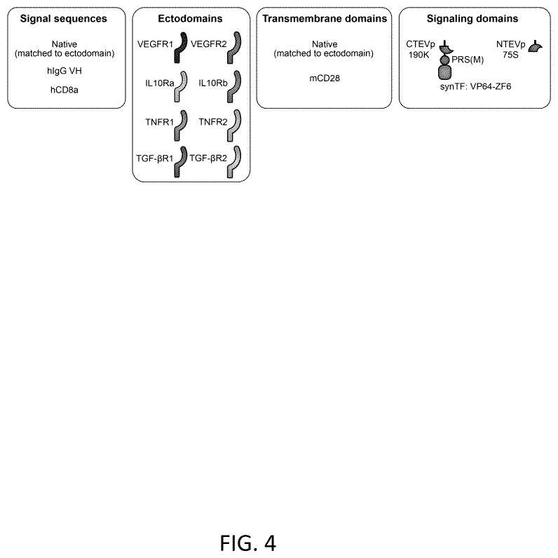

To evaluate conversion of VEGF receptors to MESA receptors, the surface expression of all variants across the full panel of design choices (FIG. 4) was measured. In general, all the receptor variants tested expressed well on the cell surface (FIG. 5B). To verify that full-length receptors were being surface expressed, whole cell expression was evaluated by western blot, and all receptors were expressed at their expected size considering post-translational modifications (FIG. 5C). VEGFR2 receptors appear larger than VEGFR1 receptors, suggesting higher levels of glycosylation. VEGFR2-based receptors exhibit higher surface and whole-cell expression levels than VEGFR1-based receptors. Signal sequence and TMD choice did not meaningfully affect receptor trafficking to the cell surface (FIG. 5B). These observations suggest that ECD choice is the biggest determinant in receptor surface expression. Given that these results did not suggest that one signal sequence was significantly better than the rest and hypothesizing that signal sequence choice would not impact signaling ability because it is cleaved in the receptor trafficking process, experiments proceeded only with receptors containing their natural signal sequence.

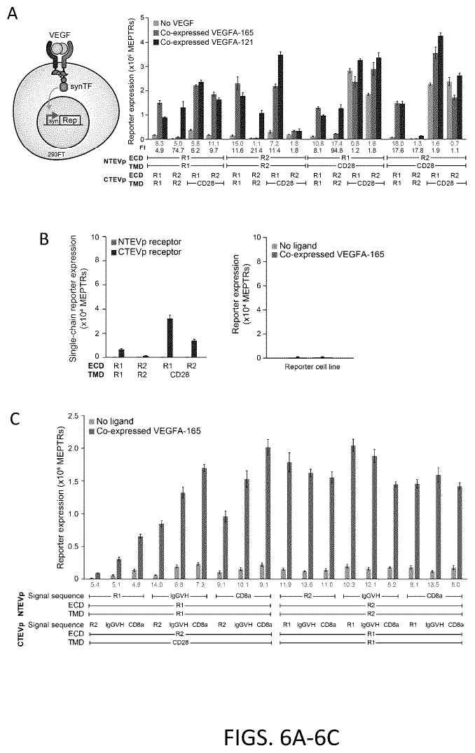

Next evaluation of whether candidate VEGF NatE MESA receptors were functional was performed. Pairwise combinations of variants that differ in ECD, TMD, and signaling domain (FIG. 6A). To quickly identify receptor combinations that are not conducive to signaling, a transient transfection approach was employed in which the target ligand is co-expressed with the receptor chains and the fluorescent reporter is genomically integrated (FIG. 5A). In this approach, both surface-expressed and intracellular receptors have access to the target ligand, thereby increasing the occurrence of binding events. Therefore, if a receptor pair does not signal in this assay setup, it will not signal when stimulated with exogenous, externally supplied ligand. Both the VEGFA165 isoform, which is the most abundant form and can be found bound to the extracellular matrix, as well as the VEGFA121 isoform, which is less abundant and is only found in soluble form, were tested. In general, the conversion of VEGFRs into MESA receptors yielded several inducible configurations that produce an increase in reporter expression when co-expressed with VEGF in both homo- and heterodimeric receptor configurations (FIGS. 6A-C). Receptor pairs in which both chains contain a CD28 TMD produced high background (ligand-independent) signal, resulting in low fold inductions. This is consistent with previous observations with MESA receptors and CARs, as the CD28 TMD has a high propensity to aggregate. Moreover, receptor pairs in which the CTEVp chain contains a VEGFR2 ECD and TMD exhibited more signaling when co-expressed with VEGFA121. This could be due to differences in geometry of binding of the different ECDs with the VEGFA variants, which can affect the interaction of the intracellular receptor parts upon ligand binding. It was also verified that changes in signal sequence choice did not affect signaling ability of receptor pairs, and it was hypothesize that observed effects upon magnitude of reporter signal are attributable to changes to receptor expression level (FIGS. 5C and 6C). Subsequent experiments focused only on sensing VEGFA165, since it is the most abundant variant and most relevant to sensing features of the TME.

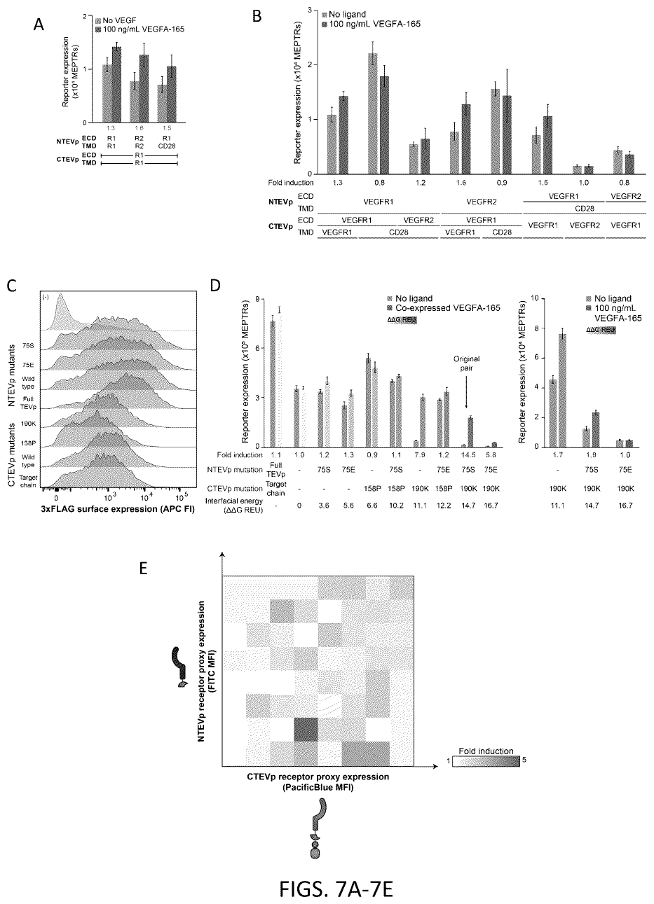

Using the most promising receptor pairs identified with strong inducible signaling in response to co-expressed ligand, signaling with exogenous (external) recombinant VEGF was tested. When receptors were transiently transfected, only a few combinations showed minimal inducibility compared to the co-expressed ligand assay (FIGS. 7A and 7B). It was hypothesized that receptors that signal with higher reporter output (i.e., reconstitute TEVp more easily and release the tethered synTF more frequently) would demonstrate better induction with exogenous ligand. Thus, new VEGFR MESA receptors were engineered containing alternative split TEVp mutants that varied in split protease reconstitution propensity (interfacial energies). It was first verified that changing the split TEVp mutants did not ablate surface expression (FIG. 7C). Their performance was then tested in response to either co-expressed and exogenous VEGF and identified one other promising receptor pair with a wildtype NTEVp domain and 190K CTEVp domain (FIG. 7D). While both pairs demonstrated high fold induction with co-expressed ligand, response to exogenous VEGF was still minimal (FIGS. 7D and 7E). It was also hypothesized that assay timing conditions could be hampering the magnitude of receptor induction with exogenous ligand. Since only receptors on the cell surface can bind to exogenous ligand, there needs to be enough time after transfection for adequate receptor production and trafficking to the cell surface as well as enough time after ligand addition to allow for signaling and sufficient reporter accumulation for detection.

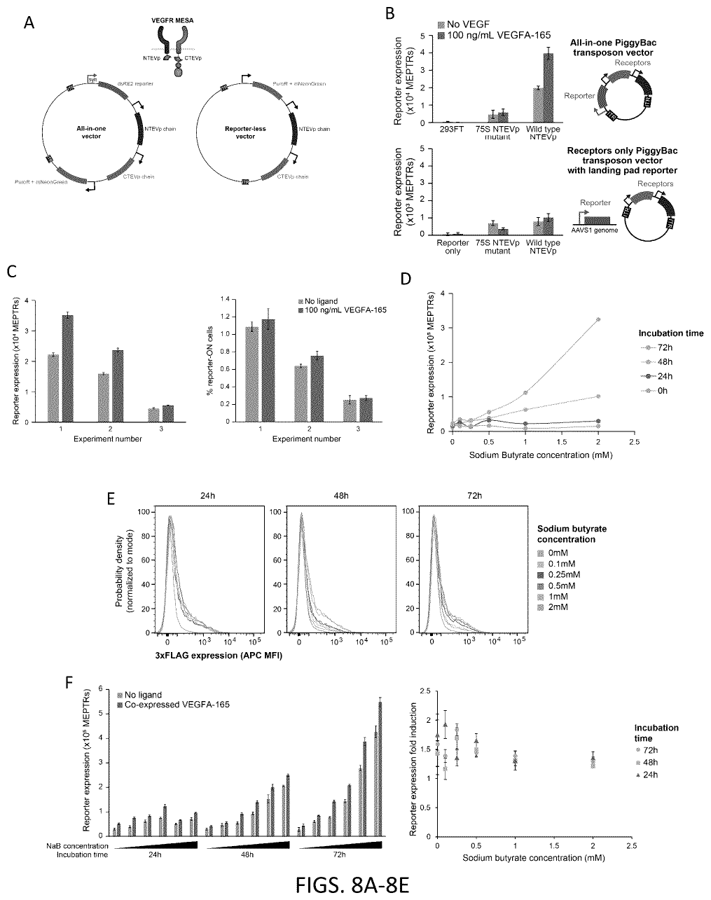

To mitigate the challenge of designing an assay to detect external ligand and to evaluate receptor performance in a context that is most translationally relevant for a cell-based therapy context, VEGFR NatE MESA receptors were stably integrated in a genomic context. Receptors were genomically integrated via transposons because of their translational relevance and because they can better accommodate the relatively large size of these receptors, around 3 kb total. Two strategies were evaluated for integrating receptor systems into the genome of HEK293FT cells: one in which the whole system (inducible reporter, NTEVp receptor chain, and CTEVp receptor chain) was contained in the same transposon, as well as one in which only the receptor chains were included and integrated into the previously engineered Landing Pad reporter cells (FIG. 8A). Stable integration of the receptors via PiggyBac transposons enabled sensing of exogenous ligand (FIG. 8B), with the “all-in-one” transposon integration strategy yielding better overall performance (fold induction) than the receptor-only transposon with a pre-integrated reporter. Because silencing is known to be a challenge with genomic integration of transgenes, this cell line was re-tested after subculturing for three weeks. When this cell line was subcultured and retested after multiple passages, a reduction in receptor signaling and fold inductions was observed as well as a reduction in the fraction of cells with an active reporter (FIG. 8C). To test the theory that silencing impacted accessibility of the integrated transposons the cells were treated with sodium butyrate (NaB), an HDAC inhibitor known to help re-open chromatin and validated for use with gene circuits in mouse and human cell lines. NaB treatment led to dose- and time-dependent reporter expression increase (FIG. 8D), as well as dose-dependent receptor surface expression increase at longer incubation times (FIG. 8E). This confirmed the hypothesis of silencing of the transgenes affecting receptor system performance and showed that initial fold inductions could be recovered by NaB treatment, with optimal fold inductions at lower NaB doses (FIG. 8F).

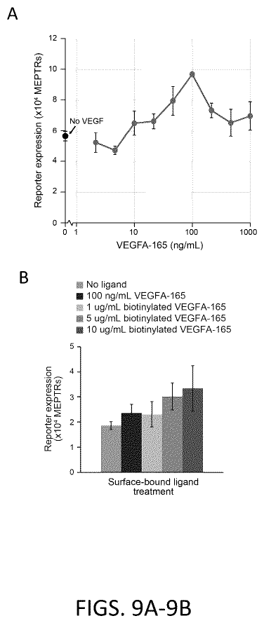

Furthermore, stable expression of receptors enabled the characterization of system properties. Firstly, it was shown that receptors can detect soluble external ligand dose-dependently (FIG. 9A), with the highest reporter signal at a dose of 100 ng/ml. It was also showed that these biosensors can detect surface-bound ligand when cultured in streptavidin coated plates treated with biotin-conjugated VEGF (FIG. 9B). Higher concentrations of ligand resulted in higher reporter signal and fold induction. Overall, these results show that the engineered VEGFR NatE MESA receptors are capable of sensing exogenous VEGF in a dose-dependent manner and in different contexts (soluble as well as surface-bound).

### Example 2—Conversion of IL-10R

IL-10 NatE MESA receptors comprise human IL10Rα and human IL10Rβ signal peptides and ectodomains (hIL10Rα SP (SEQ ID NO: 30) and ECD (SEQ ID NO: 31): aa1-225, hIL10RB SP (SEQ ID NO: 34) and ECD (SEQ ID NO: 35): aa1-220). When the native transmembrane domains were used, nine amino acids of the intracellular juxtamembrane domain were retained (hIL10Rα JMD: aa257-265 (SEQ ID NO: 33), hIL10RB JMD: aa243-251 (SEQ ID NO: 37)). In some variants, the mouse CD28 transmembrane domain was used in place of the hIL10Rα and hIL10RB transmembrane domains (hIL10Rα TMD: aa236-256 (SEQ ID NO: 32), hIL10RB TMD: aa221-242 (SEQ ID NO: 36), mCD28 TMD: aa154-174 (SEQ ID NO: 28)) with a juxtamembrane domain consisting of GGGSGG (SEQ ID NO: 29). When alternative signal peptides were used, the natural hIL10Rα and hIL10Rβ signal peptides were exchanged for the signal peptides of the human CD8a receptor and the human IgG variable heavy chain (hIL10Rα SP: aa1-21 (SEQ ID NO: 30), hIL10RB SP: aa1-19 (SEQ ID NO: 34), hCD8a SP: aa1-21 (SEQ ID NO: 23), hIgGVH SP: aa1-19 (SEQ ID NO: 24)). Sequences can be found in Tables 5 and 6.

TABLE 6
IL-10R Sequences

| | SEQ ID | |
|---|---|---|
| Name | NO | Sequence |
| IL-10Rα SP | 30 | MLPCLVVLLAALLSLRLGSDA |
| IL-10Rα ED | 31 | HGTELPSPPSVWFEAEFFHHILHWTPIPNQSESTCYEVAL |
| | | LRYGIESWNSISNCSQTLSYDLTAVTLDLYHSNGYRARV |
| | | RAVDGSRHSNWTVTNTRFSVDEVTLTVGSVNLEIHNGFI |
| | | LGKIQLPRPKMAPANDTYESIFSHFREYEIAIRKVPGNFTF |
| | | THKKVKHENFSLLTSGEVGEFCVQVKPSVASRSNKGMW |
| | | SKEECISLTRQYFTVTN |
| IL-10Rα TMD | 32 | VIIFFAFVLLLSGALAYCLAL |
| IL-10Rα JMD | 33 | QLYVRRRKK |
| IL-10Rβ SP | 34 | MAWSLGSWLGGCLLVSALG |
| IL-10Rβ ED | 35 | MVPPPENVRMNSVNFKNILQWESPAFAKGNLTFTAQYL |
| | | SYRIFQDKCMNTTLTECDFSSLSKYGDHTLRVRAEFADE |
| | | HSDWVNITFCPVDDTIIGPPGMQVEVLADSLHMRFLAPK |
| | | IENEYETWTMKNVYNSWTYNVQYWKNGTDEKFQITPQ |
| | | YDFEVLRNLEPWTTYCVQVRGFLPDRNKAGEWSEPVCE |
| | | QTTHDETVPS |
| IL-10Rβ TMD | 36 | WMVAVILMASVFMVCLALLGCF |
| IL-10Rβ JMD | 37 | ALLWCVYKK |

Surface expression of all variants across the full panel of design choices was first evaluated (FIG. 4). It was found that all variants were highly expressed on the cell surface (FIG. 10B). Selection of one of the alternative signal sequences boosted surface expression moderately compared to the native signal sequences, and this was more pronounced for receptors with an IL-10Rβ ECD. It was verified that full-length receptors were being expressed by analyzing whole cell expression via western blot, and that most receptors were expressed at similar levels within each set of NTEVp or CTEVp receptors, except for reduced expression of the IL-10Rβ receptors that retain their native signal sequence, confirming the observations of surface expression (FIG. 10C).

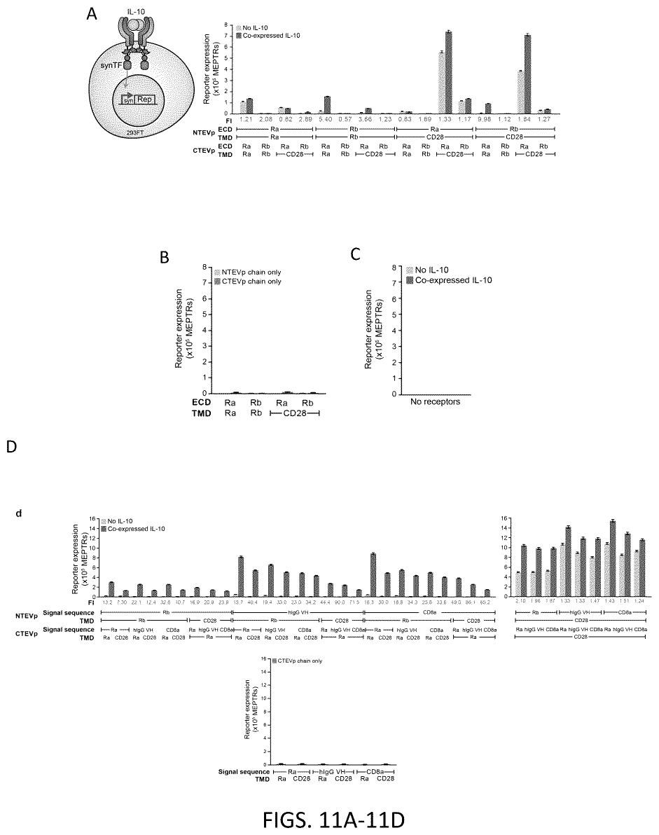

Functional performance of all different pairwise combinations of receptors with different ECs, TMDs, and signaling domains was then tested using receptors with their native signal sequences (FIG. 11A). Again a transient transfection approach was employed in which the target ligand, here human IL-10, is co-expressed with the receptors and the fluorescent reporter is genomically integrated (FIG. 10A). It was observed that across pairings of TMDs and signaling domains, receptor pairs with the same ECD demonstrated minimal ligand inducibility (fold induction close to 1). The overall magnitude of background and induced signal is higher for receptor pairs in which both ECDs are IL-10Ra, suggesting that these ECDs may mediate some pre-association. It was observed that receptor pairs with different ECDs (IL-10Rα on one receptor and IL-10Rβ on the other receptor) all demonstrated inducible signaling. The specific configuration in which the NTEVp receptor contains an IL-10RB ECD and the CTEVp receptor contains an IL-10Rα ECD demonstrated superior inducibility (higher fold induction) and overall higher induced signaling output, this configuration was selected for future analyses. It was confirmed that signaling is mediated by pairs of receptors with the ligand, and not by either receptor or transfected ligand alone (FIGS. 11B and C). Since substantial differences in expression for IL-10Rβ receptors with alternative signal sequences was observed, this selected promising ECD/signaling domain configuration across TMD and signal sequence variants was evaluated (FIG. 11D). It was confirmed that within this configuration, all pairs demonstrated ligand-inducible signaling. It was found that overall magnitude of induced signal and sometimes fold induction could be increased by employing different pairs of signal sequences on the IL-10Rβ NTEVp receptor. The CD8a signal sequence on the IL-10Rβ NTEVp receptor and the IL-10Rα receptor was selected for proceeding studies because overall magnitude of signal was highest, and it was hypothesized that this property would be useful when implementing these receptors by genomic integration and in T cells, where receptor expression level is lower. Lastly, like with the VEGF receptors, it was again observed high background signal for receptor pairs in which CD28 TMD is included on both receptors (FIGS. 11A and 11D).

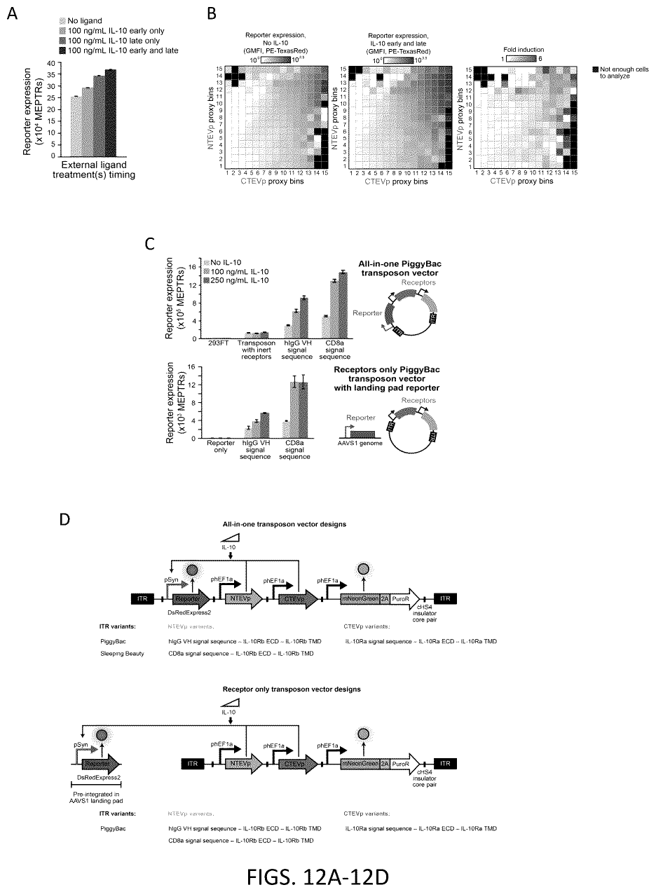

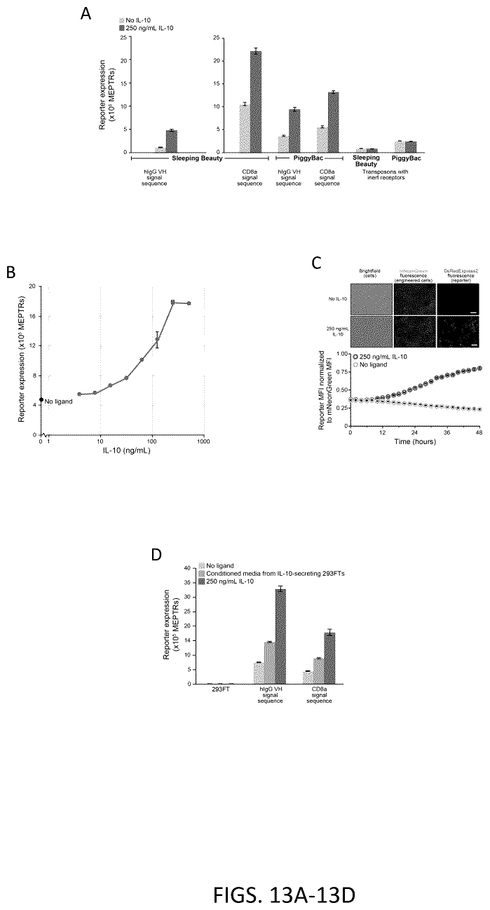

It was next evaluated whether the most promising IL-10 receptor pair could sense external ligand. Similar to the VEGFR-based NatE MESA system, minimal inducibility with external ligand was observed with transfected receptor-encoding plasmids (FIGS. 12A and B). To better characterize response to external ligand and evaluate the sensitivity of this biosensor, the receptors were genomically integrated via PiggyBac and Sleeping Beauty transposons (FIGS. 12C and D). Systems in which either just the receptors (and selection markers) were genomically integrated into the landing pad genomic reporter cell line or the receptors, selection markers, and reporter were genomically integrated from one transposon into previously un-modified cells were evaluated. Interestingly, strong inducibility was observed with external ligand across all of these designs. The induced reporter signal was strongest when the reporter was included in the same transposon as the receptors, likely because multiple copies were integrated throughout the genome (FIGS. 12C and 13A). Cells were also imaged by fluorescence microscopy after 48 h of culture with ligand to depict the increase in reporter expression above background (FIG. 13C). To characterize the dynamic range of this biosensor, cells were cultured with a range of recombinant IL-10 concentrations (FIG. 13B). The biosensor was able to distinguish IL-10 concentrations down to 16 ng/ml and saturated around 250 ng/ml. The cells were also able to sense IL-10 secreted by engineered HEK293FT cells (FIG. 13D).

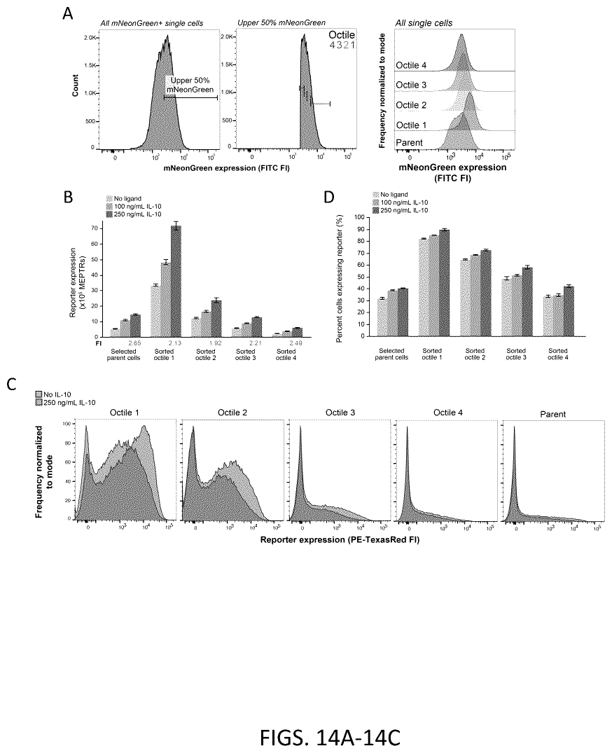

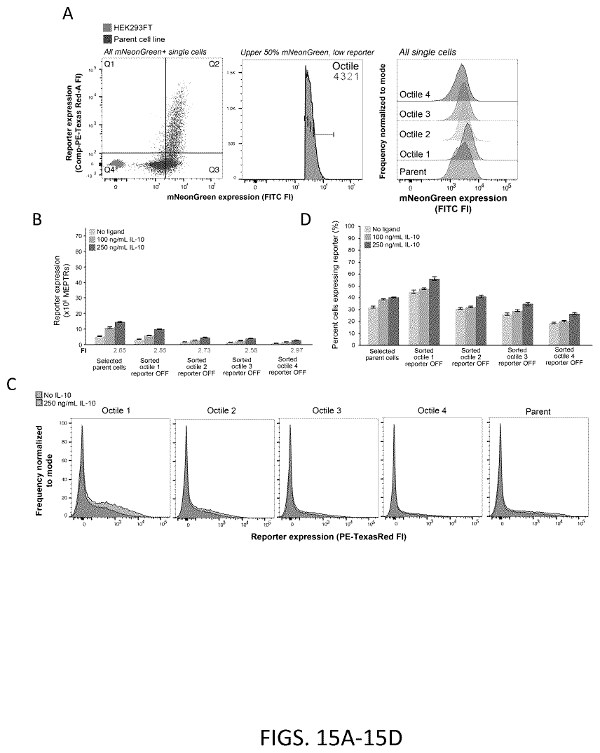

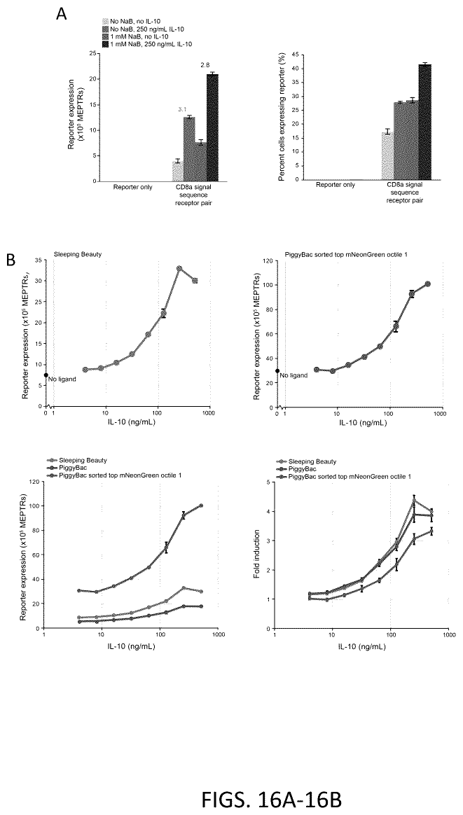

To better understand which cells are signaling within the engineered cell population and guide future implementations of this biosensor, the cells were sorted using two different strategies. First, the cells were sorted based on how much of the constitutive fluorescent protein mNeonGreen they expressed as a proxy for how many copies of the transposon were stably integrated (FIG. 14A). The top half of the population, which is where all of the reporter positive cells exist, was broken into four sections (referred to as octiles of the full population). It was found that cells with higher mNeonGreen expression did display higher background and induced signaling and fold induction was relative constant across these populations (FIGS. 14B and 14C). Interestingly, the addition of ligand only moderately increased the percent of cells exhibiting signaling (reporter-positive), suggesting that addition of ligand does not make reporter-negative cells become reporter-positive. Instead, addition of ligand increases reporter output of already reporter positive cells (FIG. 14D). It was hypothesized that cells with nonzero background (reporter positive before ligand addition) are least likely to silence the reporter gene and are responsible for most of the signaling within the population. The second sort strategy employed was designed to evaluate if cells with no background signal were capable of signaling, and if so, if a low background sort strategy could result in a higher performing population based on fold induction. The top four octiles were looked at based on mNeonGreen expression but also restricted gating to include only reporter negative cells (FIG. 15A). It was found that cells in all four octiles were still capable of signaling and again, the magnitude of signal decreased as mNeonGreen expression decreased (FIGS. 15B and 15C). Again, only moderate increases in percent of cells signaling upon ligand addition and the overall percentages were much lower suggesting that there are a higher proportion of non-signaling cells in the reporter negative cell population (FIG. 15D). To evaluate if silencing was limiting the proportion of reporter-positive cells, sodium butyrate was used on the parent (pre-sort population) and it was found that the percent of signaling cells increased, along with the magnitude of both background and induced signal (FIG. 16A). Interestingly, across all of these strategies that impact genetic context (sorting based on transposon copy number via mNeonGreen expression, sorting based on background reporter, treatment with a chromatin-modifying drug, implementation via two different transposon systems), it was found that fold induction was relatively conserved, suggesting that the functional assays are capturing performance features of the receptors themselves, independent of context (FIG. 16B). This suggests that the IL-10 biosensor is robust to genetic context and resulting expression level.

### Example 3—Conversion of TGF-βR

TGF-β NatE MESA receptors comprise human TGF-βR1 and TGF-βR2 signal peptides and ectodomains (TGF-βR1 SP (SEQ ID NO: 38) and ECD (SEQ ID NO: 39): aa1-126, TGF-βR2 SP (SEQ ID NO: 42) and ECD (SEQ ID NO: 43): aa1-166). When the native transmembrane domains are used, five amino acids of the intracellular juxtamembrane domain were retained (hTGF-βR1 JMD: aa148-152 (SEQ ID NO: 41), hTGF-βR2 JMD: aa188-192 (SEQ ID NO: 45)). In some variants, the mouse CD28 transmembrane domain was used in place of the TGF-βR1 and TGF-βR2 transmembrane domains (hTGF-βR1 TMD: aa127-147 (SEQ ID NO: 40), hTGF-βR2 TMD: aa167-187 (SEQ ID NO: 44), mCD28 TMD: aa154-174 (SEQ ID NO: 28)) with a juxtamembrane domain consisting of GGGSGG (SEQ ID NO: 29). When alternative signal peptides were used, the natural hTGF-βR1 and hTGF-βR2 signal peptides were exchanged for the signal peptides of the human CD8a receptor and the human IgG variable heavy chain (hTGF-βR1 SP: aa1-33 (SEQ ID NO: 38), hTGF-βR2 SP: aa1-22 (SEQ ID NO: 42), hCD8a SP: aa1-21 (SEQ ID NO: 23), hIgGVH SP: aa1-19 (SEQ ID NO: 24)). Sequences can be found in Tables 5 and 7.

TABLE 7
TGF-βR Sequences

| | SEQ ID | |
|---|---|---|
| Name | NO | Sequence |
| TGF-βR1 SP | 38 | MEAAVAAPRPRLLLLVLAAAAAAAAALLPGATA |
| TGF-βR1 ED | 39 | LQCFCHLCTKDNFTCVTDGLCFVSVTETTDKVIHNSM |
| | | CIAEIDLIPRDRPFVCAPSSKTGSVTTTYCCNQDHCNKI |
| | | ELPTTVKSSPGLGPVEL |
| TGF-βR1 TMD | 40 | AAVIAGPVCFVCISLMLMVYI |
| TGF-βR1 JMD | 41 | CHNRT |
| TGF-βR2 SP | 42 | MGRGLLRGLWPLHIVLWTRIAS |
| TGF-βR2 ED | 43 | TIPPHVQKSVNNDMIVTDNNGAVKFPQLCKFCDVRFS |
| | | TCDNQKSCMSNCSITSICEKPQEVCVAVWRKNDENIT |
| | | LETVCHDPKLPYHDFILEDAASPKCIMKEKKKPGETFF |
| | | MCSCSSDECNDNIIFSEEYNTSNPDLLLVIFQ |
| TGF-βR2 TMD | 44 | VTGISLLPPLGVAISVIIIFY |
| TGF-βR2 JMD | 45 | CYRVN |

Surface and whole cell expression of TGF-βR NatE MESA receptor variants was first evaluated. It was found that compared to the other natural receptor systems explored, TGF-BR-based receptors were not as well expressed on the cell surface across all tested variants (FIG. 17B). TGF-βR1-containing receptors were more highly expressed than TGF-βR2-containing receptors when native signal sequences were employed, but this flipped when alternative signal sequences were employed, especially the CD8a signal sequence (FIG. 17C). The variants with the CD8a signal sequence were then functionally validated because they were best expressed on the surface. Receptor performance was evaluated with three different ligand expression systems: pro-TGF-β or mature TGF-β with two different signal secretion sequences. In general, similar receptor behaviors with all three ligand expression systems was observed (FIG. 17D). Most receptor pairs showed no inducibility (background and induced reporter output were similar), while some were de-inducible (presence of ligand resulted in significantly less reporter output). Of note, it was found that receptor pairs with matching TGF-βR1 ECDs produced higher magnitudes of signal with and without ligand, which agrees with previous findings that suggest that this receptor can homodimerize when highly expressed, as is done in this experiment. It was also found that some pairings with both receptors containing CD28 TMDs also produced increased levels of background and induced signal, though much lower than what was observed with other systems. It was unexpected to see that none of the mixed pairs of ECDs yielded ligand-induced signaling and that none of the matched TGF-βR2 ECDs yielded ligand-induced signaling. Because no receptor pairings showed a ligand-induced increase in signaling and overall low levels of signaling, it was verified that this was not due to something about the native receptor TMD/JMD structure that caused an inability for reconstituted TEVp to cleave its recognition sequence (FIG. 17E).

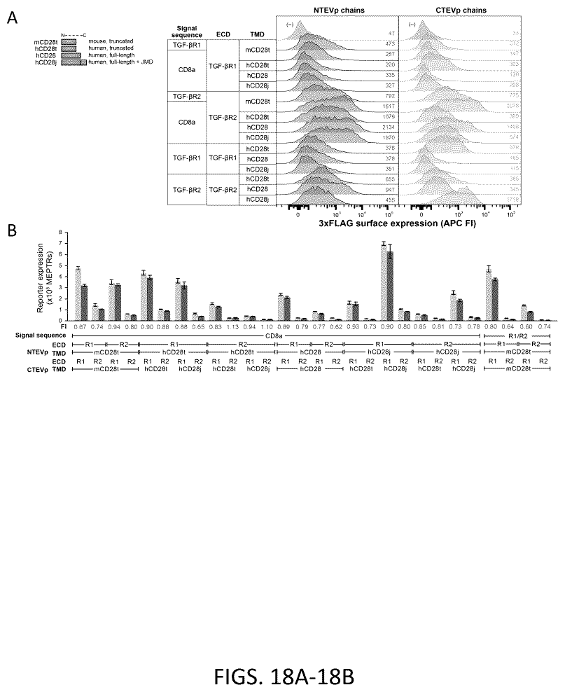

Based on these results and because expression level and particularly surface expression of these receptors was low compared to other synthetic receptor systems characterized in this study, it was evaluated whether the designs had removed critical parts of the JMD that are required for expression. Particularly, evaluation of how switching to a human CD28 TMD, extending the human CD28 TMD to be full-length, and including a human CD28 JMD might increase expression and resulting signaling by CD28-bearing receptors was performed. Overall, it was found that these extensions minimally impacted surface expression, (FIG. 18A). Functional performance was not improved because no receptor pairs were inducible, but we did observe that moving signaling domains further from the membrane by extension of JMDs only led to high levels of signal when paired with a receptor with a matching “length”, suggesting that split TEVp reconstitution is sensitive to distance from the membrane (FIG. 18B). These results suggested that ligand-mediated assembly of receptor heterotetramers and TGF-βR2 dimers is not conducive to split TEVp reconstitution and biosensor conversion. Overall, conversion of TGF-βR to a synthetic biosensor was unsuccessful in the chosen design space.

### Example 4—Conversion of TNFR

TNF NatE MESA receptors comprise human TNFR1 and TNFR2 signal peptides and ectodomains (hTNFR1 SP (SEQ ID NO: 46) and ECD (SEQ ID NO: 47): aa1-182, hTNFR2 SP (SEQ ID NO: 50) and ECD (SEQ ID NO: 51): aa1-257). When the native transmembrane domains are used, five amino acids of the intracellular juxtamembrane domain were retained (hTNFR1 JMD: aa233-237 (SEQ ID NO: 49), hTNFR2 JMD: aa288-292 (SEQ ID NO: 53)). In some variants, the mouse CD28 transmembrane domain was used in place of the TNFR1 and TNFR2 transmembrane domains (hTNFR1 TMD: aa212-232 (SEQ ID NO: 48), hTNFR2 TMD: aa258-287 (SEQ ID NO: 53), mCD28 TMD: aa154-174 (SEQ ID NO: 28)) with a juxtamembrane domain consisting of GGGSGG (SEQ ID NO: 29). When alternative signal peptides were used, the natural hTNFR1 and hTNFR2 signal peptides were exchanged for the signal peptides of the human CD8a receptor and the human IgG variable heavy chain (hTNFR1 SP: aa1-29 (SEQ ID NO: 46), hTNFR2 SP: aa1-22 (SEQ ID NO: 50), hCD8a SP: aa1-21 (SEQ ID NO: 23), hIgGVH SP: aa1-19 (SEQ ID NO: 24)). Sequences can be found in Tables 5 and 8.

TABLE 8
TNFR Sequences

| | SEQ ID | |
|---|---|---|
| Name | NO | Sequence |
| TNFR1 SP | 46 | MGLSTVPDLLLPLVLLELLVGIYPSGVIG |
| TNFR1 ED | 47 | LVPHLGDREKRDSVCPQGKYIHPQNNSICCTKCHKGTYL |
| | | YNDCPGPGQDTDCRECESGSFTASENHLRHCLSCSKCRK |
| | | EMGQVEISSCTVDRDTVCGCRKNQYRHYWSENLFQCFN |
| | | CSLCLNGTVHLSCQEKQNTVCTCHAGFFLRENECVSCSN |
| | | CKKSLECTKLCLPQIENVKGTEDSGTT |
| TNFR1 TMD | 48 | VLLPLVIFFGLCLLSLLFIGL |
| TNFR1 JMD | 49 | MYRYQ |
| TNFR2 SP | 50 | MAPVAVWAALAVGLELWAAAHA |
| TNFR2 ED | 51 | LPAQVAFTPYAPEPGSTCRLREYYDQTAQMCCSKCSPG |
| | | QHAKVFCTKTSDTVCDSCEDSTYTQLWNWVPECLSCGS |
| | | RCSSDQVETQACTREQNRICTCRPGWYCALSKQEGCRL |
| | | CAPLRKCRPGFGVARPGTETSDVVCKPCAPGTFSNTTSS |
| | | TDICRPHQICNVVAIPGNASMDAVCTSTSPTRSMAPGAV |
| | | HLPQPVSTRSQHTQPTPEPSTAPSTSFLLPMGPSPPAEGST |
| | | GD |
| TNFR2 TMD | 52 | FALPVGLIVGVTALGLLIIGVVNCVIMTQV |
| TNFR2 JMD | 53 | KKKPL |

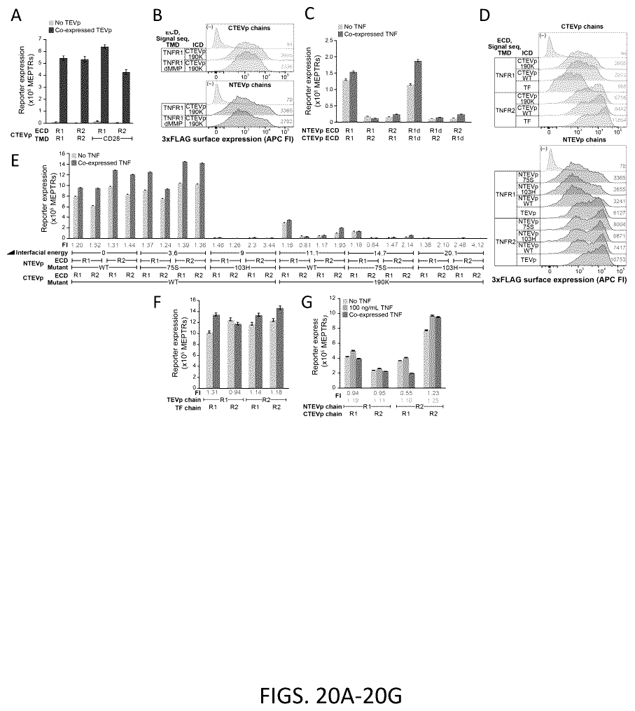

To characterize TNFR NatE MESA receptors, expression was evaluated. Strong surface expression was observed of all variants tested, and a few design choices impacted surface expression (FIG. 19B). First, variants with a TNFR2 ECD were generally more highly expressed on the surface and on a whole cell basis than variants with a TNFR1 ECD (FIGS. 19B and 19C). Secondly, variants with the respective native TMD were more highly expressed than variants with a CD28 TMD. Because signal sequence had no effect on surface expression, only variants with the respective native signal sequence for each ECD were functionally evaluated. To use a setup in which ligand is co-expressed with receptors, bioactivity was evaluated of three different ligand expression systems by transfecting them each into an engineered HEK293FT cell line that contains a previously validated genomically integrated NF-κB inducible reporter to ensure that trimers could form and initiate signaling through endogenous receptors (including TNFR1) expressed by HEK293FTs (FIG. 19D). The expression plasmid that drove the highest amount of reporter expression was selected and co-transfected along with pairwise combinations of each ECD-TMD-signaling domain variant (FIG. 19E). Minimal signaling was observed by all receptor pairs that included at least one receptor with a native TMD and we observed higher amounts of signaling by receptors pairs in which both receptors contained a CD28 TMD. It was observed that a number of the TNFR2/TNFR2 pairings produced a ligand-dependent increase in reporter expression most pronounced when both receptors contain the CD28 TMD (FIGS. 19E and 20G). These results suggested that both the ligand-unbound and ligand-bound states of receptors with native TNFR TMDs did not permit split TEVp reconstitution and signaling, while the CD28 TMD does permit signaling, though with high levels of ligand-independent background. It was verified that this lack of signaling was not due to something about the native receptor TMD/JMD structure that caused an inability for reconstituted TEVp to cleave its recognition sequence (FIG. 20A). It was also verified that proteolytic cleavage of TNFR1 by matrix metalloproteinases was not responsible for minimal signaling (FIGS. 20B and 20C). It was hypothesized that changing the split TEVp mutants to be variants that reconstitute more easily might permit more signaling by native TMD-bearing receptors. Indeed, it was found that making it easier for split TEVp components to reconstitute conferred high levels of reporter expression (both background and induced signal increased) and produced some moderately-inducible receptors (FIGS. 20D and 20E). This inducibility was independent of ECD pairing and was also observed in extended culture receptor transfection assays with recombinant, external ligand (FIG. 20G). Similarly, it was found that removing the need for reconstitution entirely by employing the MESA trans-cleavage mechanism also permitted inducible signaling with high background of some ECD pairings (FIG. 20F).

Overall, conversion of TNFRs to NatE MESA receptors yielded inducible, though low-performing receptors. Employing native transmembrane domains without split protease tuning only yielded inducible pairs with the CD28 TMD, so background signaling is high. Protease tuning yielded more inducible receptors, though background was also high. Together, these results suggest that the state change by which TNFRs signal is much less conducive to a split TEVp reconstitution and cleavage mechanism than other receptor systems explored here. It is possible that TNFRs associate in a way that does not promote split TEVp reconstitution, and signal observed with split TEVp mutants and trans-cleavage receptors is mainly determined by transient receptor encounters that change as a function of receptor cluster size. This conclusion could be supported by findings that constitutively active TNFR mutants have C-terminal domains that are held further apart in the ligand-bound state. While conversion of TNFR into a TNF-inducible synthetic biosensor was successful, the conversion process did not yield a high performing biosensor.

### Example 5—NatE-MESA Receptors can be Used to Build Therapeutic Programs for Tumor-Targeted Immunotherapies

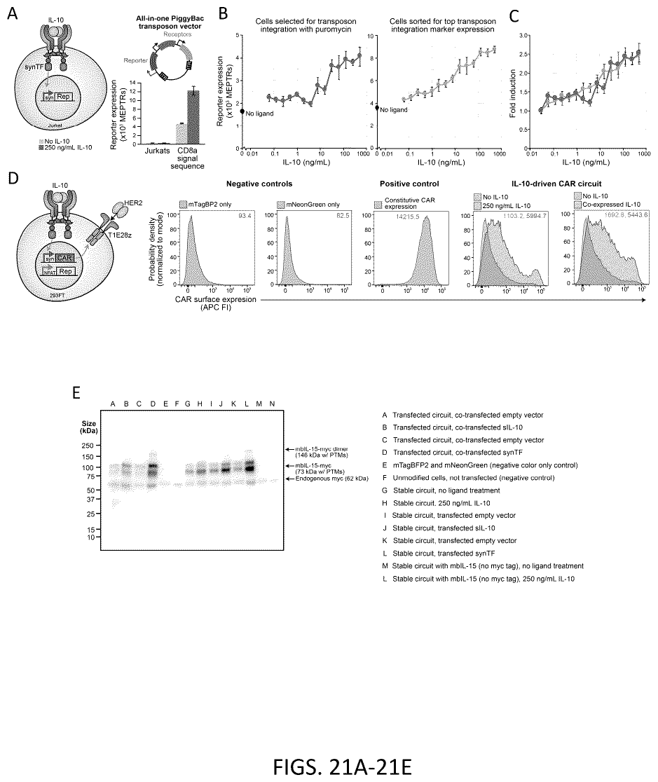

To demonstrate the translational utility of NatE MESA receptors and the conversion process described in this study, a promising biosensor from the previously described conversions was employed to build genetic programs of therapeutic relevance. Specifically, tumor-targeted immunotherapies were engineered by employing an IL-10 biosensor to detect IL-10, a tumor microenvironment cue, and respond with cytotoxic output in T cells. First, the same PiggyBac transposon system used in FIG. 12C was used to implement the biosensor, reporter, and constitutive fluorescent protein and selection marker in Jurkat T cells. It was validated that antibiotic-selected cells could sense external, recombinant IL-10 and produce reporter output and reporter output was characterized across a range of IL-10 doses (FIGS. 21A and 21B). Again a dose-dependent increase in reporter expression was observed that plateaus around 250 ng/ml IL-10 and produces a significant increase in reporter expression compared to background signal down to 16 ng/mL IL-10. As observed in HEK293FTs, sorting Jurkats for the top octile of mNeonGreen expression led to higher background and induced signal across all doses of IL-10 and did not change fold induction but did provide greater sensitivity for detection of IL-10 down to 2 ng/mL (FIG. 21C). Biosensor-driven expression of a CAR was evaluated by swapping out the fluorescent reporter gene for a gene encoding a second-generation pan-anti-ErbB CAR TIE28z. It was found that in HEK293FTs, co-expression or culture with external IL-10 led to a substantial increase in CAR surface expression (FIG. 21D), demonstrating the ability for a NatE MESA receptor to be integrated in series with other synthetic receptors like CARs to enable detection of both a soluble and surface bound cue. Similarly, biosensor-driven expression of an engineered cytokine, membrane-bound IL-15 (mbIL-15) was evaluated and an increase in expression was observed when cultured with recombinant IL-10 or transfected with co-expressed IL-10 (FIG. 21E). These results show that the IL-10 biosensor is capable of activating immunotherapy programs through synthetic transcription factor output in an IL-10-specific manner, which could be a useful capability for enhancing on-tumor specificity of CAR T cell therapies and reducing safety risks associated with constitutive expression of a pro-inflammatory cytokine.

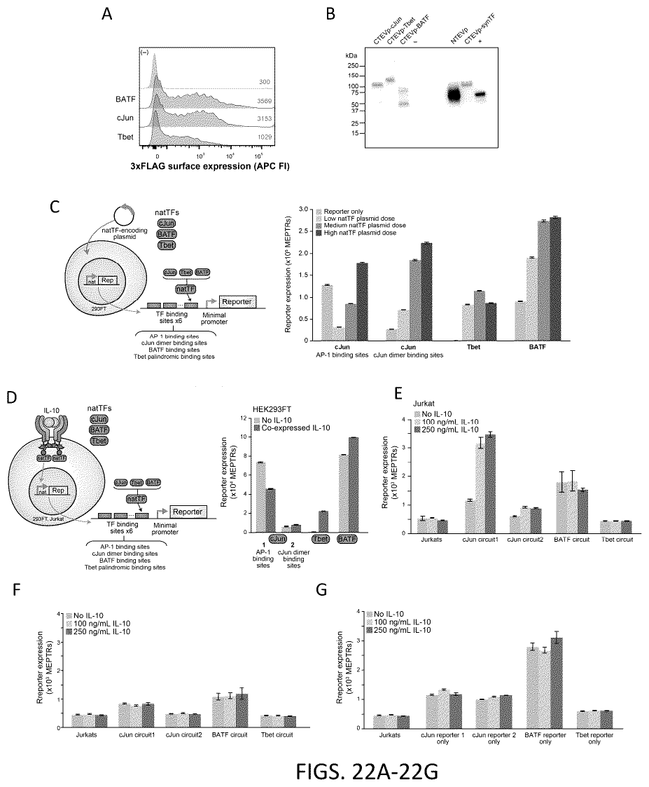

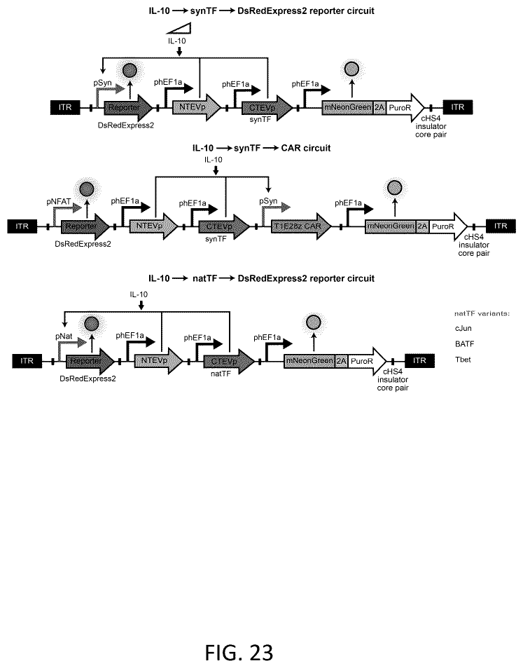

An alternative approach to using synthetic transcription factors to drive gene expression output is to implement regulation of native genes using native transcription factors. Identifying native transcription factors that can boost activation of T cells and prevent their exhaustion to improve engineered CAR T cell therapies is an active area of study. Multiple transcription factors have been identified to provide benefit to CAR T cell functions when overexpressed, including: c-Jun, which enables resistance to exhaustion and increased production of IL-2; basic leucine zipper TF ATF-like (BATF), which also enables resistance to exhaustion and promotes CD8+ T cell differentiation into effector T cells; T-box expressed in T cells (T-bet), which increases the proinflammatory anti-tumor response and promotes CD4+ T cell differentiation into a T helper 1 phenotype. It was next evaluated whether the IL-10 NatE MESA receptor system could sequester and release these native transcription factors to engineer programs that could enact these useful native programs in a tumor microenvironment-dependent manner. Overexpressing these particular transcription factors unconditionally has some oncogenic risk (c-Jun in particular) and it would be ideal to restrict their immune potentiating activity to the tumor microenvironment. The synTF was replaced with each natural TF on CTEVp-containing receptors and confirmed surface and whole cell expression (FIGS. 22A and 22B). Expression of TF-containing receptors generally decreased as TF size increased. To evaluate if functional natural TFs could be released in an IL-10-dependent manner, synthetic reporter systems were developed for each TF by engineering binding site arrays for each TF and pairing them with a YB_TATA minimal promoter and a DsRedExpress2 reporter gene (FIG. 22C). Reporter output was evaluated in response to increasing doses of TF-encoding plasmids in HEK293FTs and observed dose-dependent increases in reporter expression. Background from transfected reporter alone was variable across the reporters because HEK293FTs express basal levels of c-Jun and BATF but not T-bet, and some reporters are subject to crosstalk with other TFs expressed in HEK293FTs (Human protein atlas). Reporter expression was evaluated when natural TF-containing receptors were co-transfected with or without IL-10 and observed an increase in reporter expression with IL-10 for all three systems (FIG. 22D). This system was also evaluated when stably integrated via Piggy Bac transposons in Jurkats and found that modest ligand-induced increases in reporter expression for c-Jun but not T-bet could be detected (FIGS. 22E-22G), likely because expression level of the receptors and reporter is much lower in Jurkats and silencing is more prevalent. Ultimately, these results show that NatE MESA receptors can be designed to regulate native gene expression through the release of different types of transcription factors from tethering at the membrane. It is still an open question as to whether release of natural TFs from NatE MESA receptors can provide an adequate signal magnitude to confer meaningful effects from native gene regulation. Detailed descriptions of the transposons implemented in Jurkats to perform the assays described here are shown in FIG. 23.

### Example 6—NatE-MESA Receptors can Integrate Information about Multiple Tumor Microenvironment Cues

In the context of cell-based therapies, being able to integrate more than one input can confer several therapeutic advantages. These include robustness against varying TME cytokine signatures as well as reduced on-target, off-tumor toxicity. Other synthetic receptor systems have been shown to perform logical functions for integration of several inputs, but this has not been achieved with soluble ligands in the tumor microenvironment. Receptors that signal through synthetic TF output are particularly well-suited for many types of downstream integration and circuitry. The two most promising NatE MESA receptor systems developed (VEGFR and IL-10R) were therefore tested to determine whether they could be multiplexed in parallel to perform logical functions for detection of the TME. Previous attempts to multiplex MESA receptors have failed to create a synergistic output, likely due to both receptor and synthetic promoter characteristics. It was hypothesized that these new high-performing receptors combined with highly customizable transcription factors and cognate promoters, could yield a synergistic output.

Before developing any logic gates, the cross-reactivity of the engineered VEGF and IL-10 receptor systems was probed. It was found that only the VEGFR NTEVp chains showed some activity when paired with the IL-10R CTEVp chains (FIGS. 24A and B). This cross-reactivity was reduced in the presence of ligand, indicating that receptor dimerization of matched receptor chain pairs could inhibit transient interactions between mismatched receptor chain pairs.

Next both VEGFR and IL-10R NatE MESA receptor pairs were tested in the context of an OR gate, which is defined as a system showing a response when either (or both) of the inputs are present, and no response when neither of the inputs are present (FIG. 24C). To implement an OR gate with receptors, both receptor systems were designed to release the same synTF upon ligand binding, which activates the expression of the fluorescent output. It was observed that reporter expression was induced by either of the ligands, as well as both ligands together (FIG. 24D). The magnitude of reporter expression was determined by the receptor system being activated, with IL-10R inducing higher levels of fluorescence. This trend follows observations from individual receptor system experiments, where the IL-10R system showed higher reporter expression levels than the VEGFR system. Choice of IL-10R pair does not seem to affect the function of the OR gate, and choice of VEGFR pair only slightly affects the magnitudes of no ligand and VEGF-only conditions (FIG. 24E). These observations follow previously observed trends, in which choosing an NTEVp mutant with lower interfacial energy increases the total signal without affecting reporter fold induction.

Next an AND gate was built with NatE MESA receptors. In logic theory, an AND gate is defined as a system showing a synergistic output upon the presence of both inputs (i.e., the output with both inputs is larger than the sum of the output with each individual input). The first AND gate architecture designed involved each receptor system releasing a different synTF, which can bind to a hybrid promoter containing binding sites for both synTFs (FIG. 25A). Several promoter architectures were designed based on previous work, and tested them for synergy with soluble transfected synTFs (FIG. 25B). Even though they all displayed synergy, we moved forward only with P2 and P4, which had a combination of high synergy with soluble synTFs and high reporter output. To allow for quantification of each receptor system individually, multi-gene expression vectors (MGEVs) were created for each system, including the NTEVp and CTEVp receptors, each driven by a separate constitutive promoter, as well as a constitutive fluorescent proxy (FIG. 25C). This setup resulted in approximately 1:1 expression of each receptor (NTEVp and CTEVp) within each system, which is favorable since it was previously observed that the best fold inductions were achieved at high expression of each (FIGS. 7E and 12B). MGEVs were transfected into the reporter cell lines stably expressing the P2 and P4 promoters, along with their corresponding co-expressed ligands. With this setup, all combinations of receptor system, synTF, and cognate promoter yielded a synergistic output (FIG. 25D). In general, P4 generated better synergy than P2; we hypothesize that this is due to the mechanism of cooperative recruitment of transcriptional machinery of these synTFs. Since P2 has more binding sites for the synTFs, these can induce a higher degree of transcriptional activation, even when there is only one ligand present, which increases single-input signal. This phenomenon is heightened for the receptor system that releases the synTF whose binding sites are closer to the minimal promoter (in this case, ZF6). Interestingly, choice of receptor design within each receptor system seems inconsequential to AND gate performance, which is mostly driven by downstream elements such as synTF choice and promoter design (FIG. 25E).

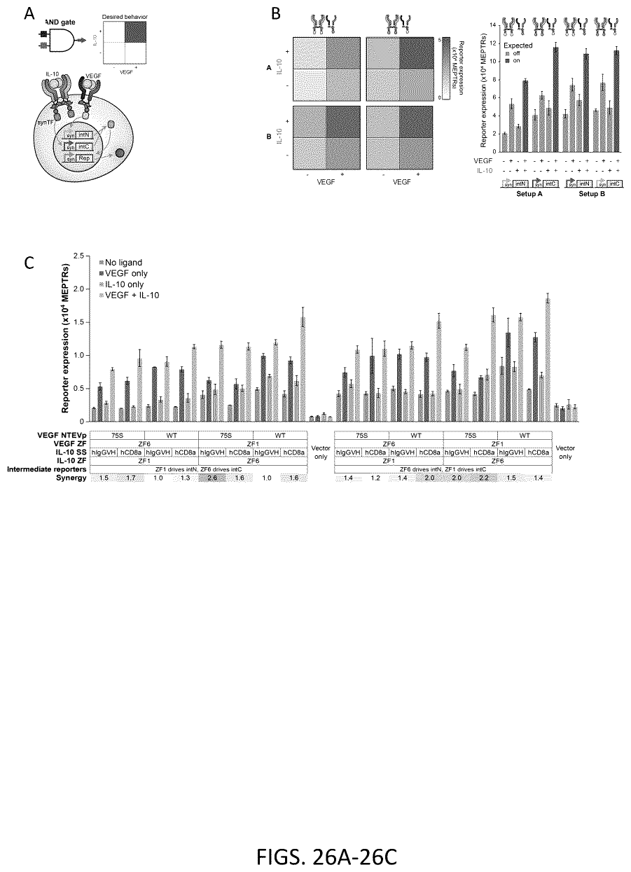

Next, a different AND gate strategy was implemented to reduce single-ligand signaling and improve system synergy by making the output from a single system signaling incapable of producing reporter output. In this case, it was desired to implement split intein domains to separate a synTF into its activation and DNA-binding domains. Each receptor system would release a different synTF (ZF1 or ZF6), which would then induce the expression of one half of the split-synTF in the presence of their corresponding ligands (FIG. 26A). To implement this, new reporter cell lines were engineered with the intermediate reporters expressing the split-synTF halves. The same MGEVs were used to transfect these cells with the corresponding co-expressed ligands. This system also showed synergy with some combinations, though the overall performance was worse than the previous system (FIG. 26B). In general, the IL-10 only signal was reduced successfully, but the VEGF only signal was not lowered. We hypothesized this was due to high ligand-independent background from the IL-10R system as compared to the VEGFR system, which caused an excess of the IL-10R-driven split-synTF half that in turn contributed to VEGF-only signaling. Because the promoter controlling the reporter was designed to be responsive to only one synTF, more synTFs can bind closer together to invoke cooperativity and create higher background signal than what was observed with a hybrid promoter. Again, receptor design choices did not affect AND gate performance as much as downstream circuit design choices (FIG. 26C). These results combined with the previous AND gate topology results reinforce the idea that level matching receptor output levels (both background and induced signal) is important for successfully achieving the desired logical function. We hypothesize that reducing background from the IL-10R-based system would increase AND gate synergy here.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

All patents, patent applications, provisional applications, and publications referred to or cited herein are incorporated by reference in their entirety, including all figures and tables, to the extent they are not inconsistent with the explicit teachings of this specification.

1. A biosensor, comprising a protein dimer comprising a first protein and a second protein that each comprise:
(a) an extracellular ligand-binding domain of a human receptor protein,
(b) a transmembrane domain,
(c) a juxtamembrane domain comprising 5-12 amino acids connected to a cytoplasmic end of the transmembrane domain, and
(d) an intracellular dimerizing domain;
wherein the intracellular dimerizing domain of the first protein comprises a half of a split protease; and
wherein the intracellular dimerizing domain of the second protein comprises (i) a complementary half of the split protease, (ii) a protease cleavage site (PCS), and (iii) a transcription factor linked thereto, such that the split protease components reconstitute upon dimerization of the first protein and the second protein, cleaving the PCS and releasing the transcription factor.

2. The biosensor of claim 1, wherein the first protein, the second protein, or both further comprise a signal peptide of the human receptor protein, which is, optionally, derived from a human CD8a receptor or a human IgG variable heavy chain.

3. The biosensor of claim 1, wherein the extracellular domain of the human receptor protein, the transmembrane domain, and the juxtamembrane domain are all derived from the same human protein.

4. The biosensor of claim 1, wherein the extracellular domain of the human receptor protein, the transmembrane domain, and the juxtamembrane domain are derived from at least two different human proteins.

5. The biosensor of claim 1, wherein the transmembrane domain is derived from a murine or human CD28 receptor.

6. The biosensor of claim 1, wherein the juxtamembrane domain comprises a flexible repeated sequence of glycine and serine amino acids.

7. The biosensor of claim 1, wherein the extracellular domain of the human receptor protein binds to transforming growth factor beta (TGF-β), a tumor necrosis factor (TNF), an interleukin, or vascular endothelial growth factor (VEGF).

8. The biosensor of claim 1, where the extracellular domain of the human receptor protein is an extracellular domain of TGF-β receptor 1 (TGF-βR1) or TGF-β receptor 2 (TGF-βR2).

9. The biosensor of claim 1, where the extracellular domain of the human receptor protein is an extracellular domain of TNF receptor 1 (TNFR1) or TNF receptor 2 (TNFR2).

10. The biosensor of claim 1, where the extracellular domain of the human receptor protein is an extracellular domain of interleukin-10 receptor b (IL-10Rb) or interleukin-10 receptor a (IL-10Ra).

11. The biosensor of claim 1, where the extracellular domain of the human receptor protein is an extracellular domain of VEGF receptor 1 (VEGFR1) or VEGF receptor 2 (VEGFR2).

12. The biosensor of claim 1, wherein the first protein comprises the N-terminal half of split tobacco etch virus protease and the second protein comprises the complementary C-terminal half of split tobacco etch virus protease, a protease cleavage site (PCS), and a transcription factor.

13. The biosensor of claim 1, wherein the first protein comprises the C-terminal half of split tobacco etch virus protease and the second protein comprises the complementary N-terminal half of split tobacco etch virus protease, a protease cleavage site (PCS), and a transcription factor.

14. The biosensor of claim 12, wherein the N-terminal half of split tobacco etch virus protease comprises SEQ ID NO: 1, 3, 5, or 6.

15. The biosensor of claim 12, wherein the C-terminal half of split tobacco etch virus protease comprises SEQ ID NO: 2, 4, or 7.

16. The biosensor of claim 1, wherein the transcription factor is a synthetic transcription (synTF) factor or a naturally occurring transcription factor.
